(19) United States
(12) Patent Application Publication
(10) Pub. No.: US 2009/0142777 A1
(43) Pub. Date: Jun. 4, 2009

(54) Reagents for the detection of protein phosphorylation in leukemia signaling pathways

(75) Inventors: Valerie Goss, Seabrook, NH (US); Albrecht Moritz, Salem, MA (US); Ting-Lei Gu, Woburn, MA (US); Kimberly Lee, Seattle, WA (US); Roberto Polakiewicz, Lexington, MA (US)

Correspondence Address: Simona Levi-Minzi, Ph.D.;General Counsel, CELL SIGNALING TECHNOLOGY, INC., 3 Trask Lane, Danvers, MA, 01923, US

(73) Assignee: CELL SIGNALING TECHNOLOGY, INC.

(21) Appl. No.: 11/973,019

(22) Filed: Oct. 5, 2007

(30) Foreign Application Priority Data
Jan. 12, 2006 (US) PCT/US06/00979

(51) Int. Cl. G01N 33/573; G01N 33/566; C07K 16/18; C07K 14/00; C12N 5/06

(52) U.S. Cl. 435/7.4; 436/501; 530/387.7; 530/350; 435/330

(57) ABSTRACT

The invention discloses 424 novel phosphorylation sites identified in signal transduction proteins and pathways underlying human Leukemia, and provides phosphorylation-site specific antibodies and heavy-isotope labeled peptides (AQUA peptides) for the selective detection and quantification of these phosphorylated sites/proteins, as well as methods of using the reagents for such purpose. Among the phosphorylation sites identified are sites occurring in the following protein types: Adaptor/Scaffold proteins, Cytoskeletal proteins, Cellular Metabolism enzymes, G Protein/GTPase Activating/Guanine Nucleotide Exchange Factor proteins, Immunoglobulin Superfamily proteins, Inhibitor proteins, Lipid Kinases, Nuclear DNA Repair/RNA Binding/Transcription proteins, Serine/Threonine Protein Kinases, Tyrosine Kinases, Protein Phosphatases, and Translation/Transporter proteins.

### FIELD OF THE INVENTION

The invention relates generally to antibodies and peptide reagents for the detection of protein phosphorylation, and to protein phosphorylation in cancer.

### BACKGROUND OF THE INVENTION

The activation of proteins by post-translational modification is an important cellular mechanism for regulating most aspects of biological organization and control, including growth, development, homeostasis, and cellular communication. Protein phosphorylation, for example, plays a critical role in the etiology of many pathological conditions and diseases, including cancer, developmental disorders, autoimmune diseases, and diabetes. Yet, in spite of the importance of protein modification, it is not yet well understood at the molecular level, due to the extraordinary complexity of signaling pathways, and the slow development of technology necessary to unravel it.

Protein phosphorylation on a proteome-wide scale is extremely complex as a result of three factors: the large number of modifying proteins, e.g. kinases, encoded in the genome, the much larger number of sites on substrate proteins that are modified by these enzymes, and the dynamic nature of protein expression during growth, development, disease states, and aging. The human genome, for example, encodes over 520 different protein kinases, making them the most abundant class of enzymes known. See Hunter, Nature 411: 355-65 (2001). Most kinases phosphorylate many different substrate proteins, at distinct tyrosine, serine, and/or threonine residues. Indeed, it is estimated that one-third of all proteins encoded by the human genome are phosphorylated, and many are phosphorylated at multiple sites by different kinases. See Graves et al., Pharmacol. Ther. 82:111-21 (1999).

Many of these phosphorylation sites regulate critical biological processes and may prove to be important diagnostic or therapeutic targets for molecular medicine. For example, of the more than 100 dominant oncogenes identified to date, 46 are protein kinases. See Hunter, supra. Understanding which proteins are modified by these kinases will greatly expand our understanding of the molecular mechanisms underlying oncogenic transformation. Therefore, the identification of, and ability to detect, phosphorylation sites on a wide variety of cellular proteins is crucially important to understanding the key signaling proteins and pathways implicated in the progression of diseases like cancer.

One form of cancer in which underlying signal transduction events are involved, but still poorly understood, is leukemia. Leukemia is a malignant disease of the bone marrow and blood, characterized by abnormal accumulation of blood cells, and is divided in four major categories. An estimated 33,500 new cases of leukemia will be diagnosed in the U.S. alone this year, affecting roughly 30,000 adults and 3,000 children, and close to 24,000 patients will die from the disease (Source: The Leukemia & Lymphoma Society (2004)). Depending of the cell type involved and the rate by which the disease progresses it can be defined as acute or chronic myelogenous leukemia (AML or CML), or acute and chronic lymphocytic leukemia (ALL or CLL). The acute forms of the disease rapidly progress resulting in the accumulation of immature, functionless cells in the marrow and blood, resulting in anemia, immunodeficiency and coagulation deficiencies, respectively. Chronic forms of leukemia progress more slowly, allowing a greater number of mature, functional cells to be produced, which amass to high concentration in the blood over time.

More than half of adult leukemias occur in patients 67 years of age or older, and leukemia accounts for about 30% of all childhood cancers. The most common type of adult leukemia is acute myelogenous leukemia (AML), with an estimated 11,920 new cases annually. Without treatment patients rarely survive beyond 6-12 months, and despite continued development of new therapies, it remains fatal in 80% of treated patients (Source: The Leukemia & Lymphoma Society (2004)). The most common childhood leukemia is acute lymphocytic leukemia (ALL), but it can develop at any age. Chronic lymphocytic leukemia (CLL) is the second most prevalent adult leukemia, with approximately 8,200 new cases of CLL diagnosed annually in the U.S. The course of the disease is typically slower than acute forms, with a five-year relative survival of 74%. Chronic myelogenous leukemia (CML) is less prevalent, with about 4,600 new cases diagnosed each year in the U.S., and is rarely observed in children.

Most varieties of leukemia are generally characterized by genetic alterations associated with the etiology of the disease, and it has recently become apparent that, in many instances, such alterations (chromosomal translocations, deletions or point mutations) result in the constitutive activation of protein kinase genes, and their products, particularly tyrosine kinases. The most well known alteration is the oncogenic role of the chimeric BCR-Abl gene, which is generated by translocation of chromosome 9 to chromosome 22, creating the so-called Philadelphia chromosome characteristic of CML (see Nowell, Science 132: 1497 (1960)). The resulting BCR-Abl kinase protein is constitutively active and elicits characteristic signaling pathways that have been shown to drive the proliferation and survival of CML cells (see Daley, Science 247: 824-830 (1990); Raitano et al., Biochim. Biophys. Acta. December 9; 1333 (3): F201-16 (1997)). The recent success of Imanitib (also known as ST1571 or Gleevec®), the first molecularly targeted compound designed to specifically inhibit the tyrosine kinase activity of BCR-Abl, provided critical confirmation of the central role of BCR-Abl signaling in the progression of CML (see Schindler et al., Science 289: 1938-1942 (2000); Nardi et al., Curr. Opin. Hematol. 11: 35-43 (2003)).

The success of Gleevec® now serves as a paradigm for the development of targeted drugs designed to block the activity of other tyrosine kinases known to be involved in leukemias and other malignancies (see, e.g., Sawyers, Curr. Opin. Genet. Dev. February; 12(1): 111-5 (2002); Druker, Adv. Cancer Res. 91:1-30 (2004)). For example, recent studies have demonstrated that mutations in the FLT3 gene occur in one third of adult patients with AML. FLT3 (Fms-like tyrosine kinase 3) is a member of the class III receptor tyrosine kinase (RTK) family including FMS, platelet-derived growth factor receptor (PDGFR) and c-KIT (see Rosnet et al., Crit. Rev. Oncog. 4: 595-613 (1993). In 20-27% of patients with AML, an internal tandem duplication in the juxta-membrane region of FLT3 can be detected (see Yokota et al., Leukemia 11: 1605-1609 (1997)). Another 7% of patients have mutations within the active loop of the second kinase domain, predominantly substitutions of aspartate residue 835 (D835), while additional mutations have been described (see Yamamoto et al., Blood 97: 2434-2439 (2001); Abu-Duhier et al., Br. J. Haematol. 113: 983-988 (2001)). Expression of mutated FLT3 receptors results in constitutive tyrosine phosphorylation of FLT3, and subsequent phosphorylation and activation of downstream molecules such as STAT5, Akt and MAPK, resulting in factor-independent growth of hematopoietic cell lines.

Altogether, FLT3 is the single most common activated gene in AML known to date. This evidence has triggered an intensive search for FLT3 inhibitors for clinical use leading to at least four compounds in advanced stages of clinical development, including: PKC412 (by Novartis), CEP-701 (by Cephalon), MLN518 (by Millenium Pharmaceuticals), and SU5614 (by Sugen/Pfizer) (see Stone et al., Blood (in press)(2004); Smith et al., Blood 103: 3669-3676 (2004); Clark et al., Blood 104: 2867-2872 (2004); and Spiekerman et al., Blood 101: 1494-1504 (2003)).

There is also evidence indicating that kinases such as FLT3, c-KIT and Abl are implicated in some cases of ALL (see Cools et al., Cancer Res. 64: 6385-6389 (2004); Hu, Nat. Genet. 36: 453-461 (2004); and Graux et al., Nat. Genet. 36: 1084-1089 (2004)). In contrast, very little is know regarding any causative role of protein kinases in CLL, except for a high correlation between high expression of the tyrosine kinase ZAP70 and the more aggressive form of the disease (see Rassenti et al., N. Eng. J. Med. 351: 893-901 (2004)).

Despite the identification of a few key molecules involved in progression of leukemia, the vast majority of signaling protein changes underlying this disease remains unknown. There is, therefore, relatively scarce information about kinase-driven signaling pathways and phosphorylation sites relevant to the different types of leukemia. This has hampered a complete and accurate understanding of how protein activation within signaling pathways is driving these complex cancers. Accordingly, there is a continuing and pressing need to unravel the molecular mechanisms of kinase-driven oncogenesis in leukemia by identifying the downstream signaling proteins mediating cellular transformation in this disease. Identifying particular phosphorylation sites on such signaling proteins and providing new reagents, such as phospho-specific antibodies and AQUA peptides, to detect and quantify them remains particularly important to advancing our understanding of the biology of this disease.

Presently, diagnosis of leukemia is made by tissue biopsy and detection of different cell surface markers. However, misdiagnosis can occur since some leukemia cases can be negative for certain markers, and because these markers may not indicate which genes or protein kinases may be deregulated. Although the genetic translocations and/or mutations characteristic of a particular form of leukemia can be sometimes detected, it is clear that other downstream effectors of constitutively active kinases having potential diagnostic, predictive, or therapeutic value, remain to be elucidated. Accordingly, identification of downstream signaling molecules and phosphorylation sites involved in different types of leukemia and development of new reagents to detect and quantify these sites and proteins may lead to improved diagnostic/prognostic markers, as well as novel drug targets, for the detection and treatment of this disease.

### SUMMARY OF THE INVENTION

The invention discloses 424 novel phosphorylation sites identified in signal transduction proteins and pathways underlying huma Leukemias and provides new reagents, including phosphorylation-site specific antibodies and AQUA peptides, for the selective detection and quantification of these phosphorylated sites/proteins. Also provided are methods of using the reagents of the invention for the detection and quantification of the disclosed phosphorylation sites.

### BRIEF DESCRIPTION OF THE DRAWINGS

FIG. 1—Is a diagram broadly depicting the immunoaffinity isolation and mass-spectrometric characterization methodology (IAP) employed to identify the novel phosphorylation sites disclosed herein.

FIG. 2—Is a table (corresponding to Table 1) enumerating the Leukemia signaling protein phosphorylation sites disclosed herein: Column A=the name of the parent protein; Column B=the SwissProt accession number for the protein (human sequence); Column C=the protein type/classification; Column D=the tyrosine or serine residue (in the parent protein amino acid sequence) at which phosphorylation occurs within the phosphorylation site; Column E=the phosphorylation site sequence encompassing the phosphorylatable residue (residue at which phosphorylation occurs (and corresponding to the respective entry in Column D) appears in lowercase; Column F=the type of leukemia in which the phosphorylation site was discovered; and Column G=the cell type(s) in which the phosphorylation site was discovered.

FIG. 3—is an exemplary mass spectrograph depicting the detection of the tyrosine 105 phosphorylation site in NCK1 (see Row 48 in FIG. 2/Table 1), as further described in Example 1 (red and blue indicate ions detected in MS/MS spectrum); Y* indicates the phosphorylated tyrosine (shown as lowercase “y” in FIG. 2).

FIG. 4—is an exemplary mass spectrograph depicting the detection of the tyrosine 292 phosphorylation site in Tyk2 (see Row 367 in FIG. 2/Table 1), as further described in Example 1 (red and blue indicate ions detected in MS/MS spectrum); Y* indicates the phosphorylated tyrosine (shown as lowercase “y” in FIG. 2).

FIG. 5—is an exemplary mass spectrograph depicting the detection of the serine 585 phosphorylation site in MARK2 (see Row 343 in FIG. 2/Table 1), as further described in Example 1 (red and blue indicate ions detected in MS/MS spectrum); S* indicates the phosphorylated serine (shown as lowercase “s” in FIG. 2).

FIG. 6—is an exemplary mass spectrograph depicting the detection of the tyrosine 187 phosphorylation site in BLK (see Row 356 in FIG. 2/Table 1), as further described in Example 1 (red and blue indicate ions detected in MS/MS spectrum); Y* indicates the phosphorylated tyrosine (shown as lowercase “y” in FIG. 2).

FIG. 7—is an exemplary mass spectrograph depicting the detection of the tyrosine 842 phosphorylation site in FLT3 (see Row 370 in FIG. 2/Table 1), as further described in Example 1 (red and blue indicate ions detected in MS/MS spectrum); Y* indicates the phosphorylated tyrosine (shown as lowercase “y” in FIG. 2).

FIG. 8—is an exemplary mass spectrograph depicting the detection of the tyrosine 27 phosphorylation site in Tel (see Row 303 in FIG. 2/Table 1), as further described in Example 1 (red and blue indicate ions detected in MS/MS spectrum); Y* indicates the phosphorylated tyrosine (shown as lowercase “y” in FIG. 2).

FIG. 9—is an exemplary mass spectrograph depicting the detection of the tyrosine 211 phosphorylation site in eIF4B (see Row 397 in FIG. 2/Table 1), as further described in Example 1 (red and blue indicate ions detected in MS/MS spectrum); Y* indicates the phosphorylated tyrosine (shown as lowercase “y” in FIG. 2).

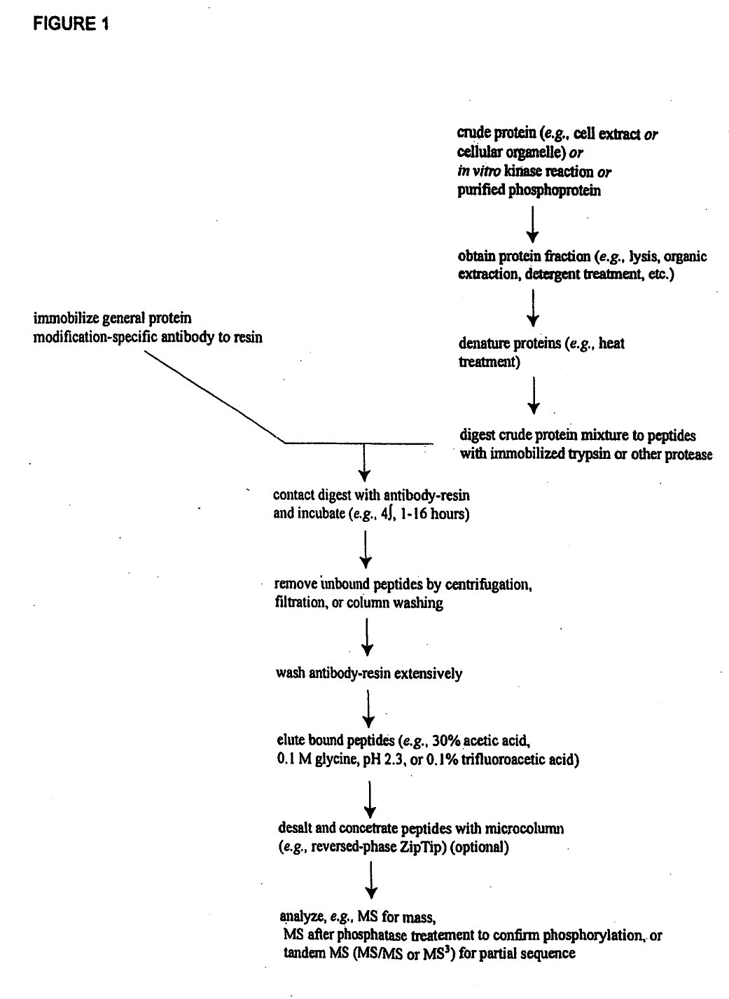

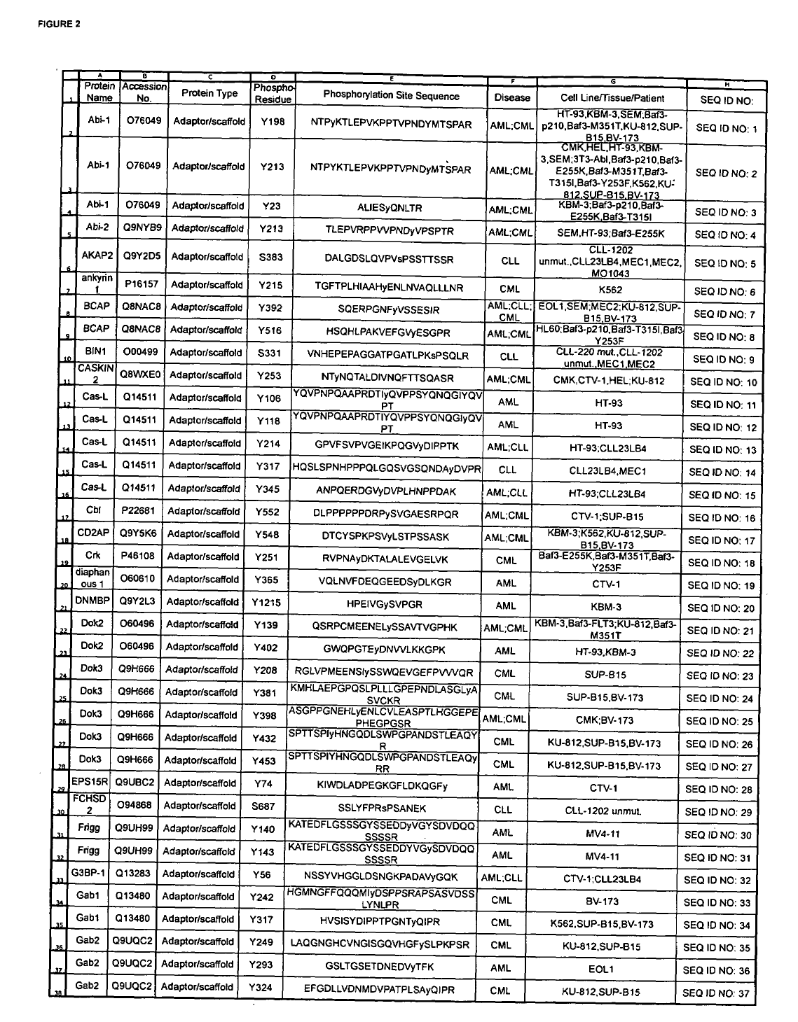

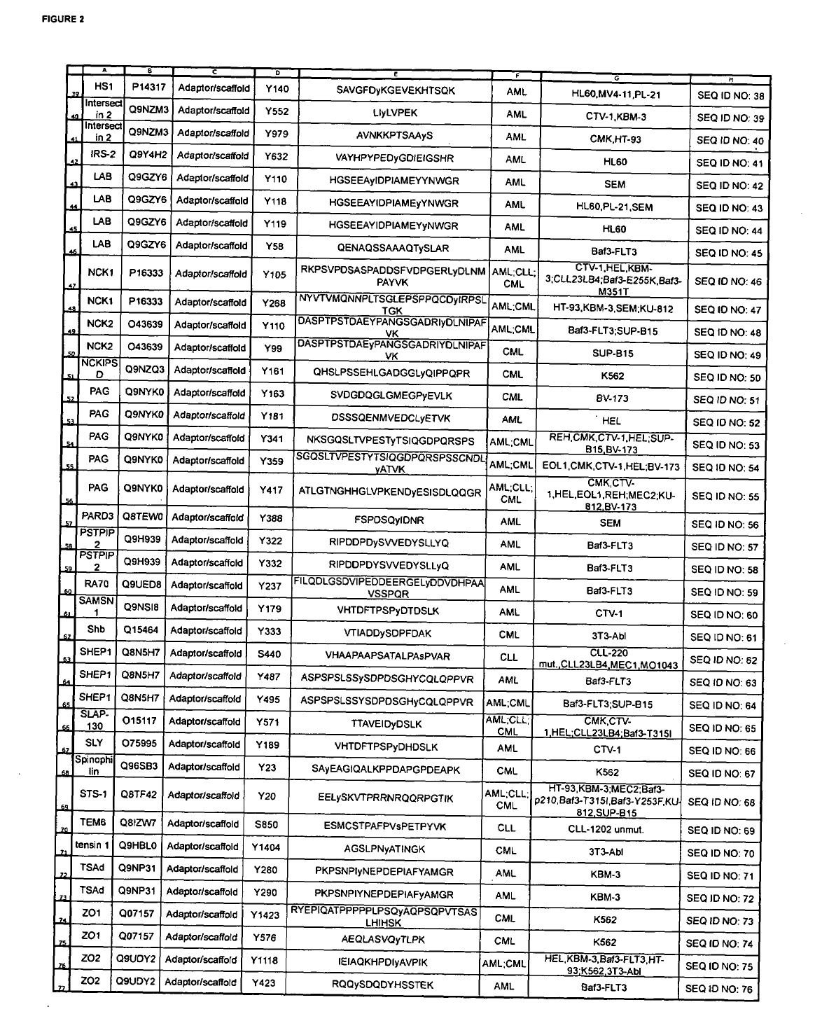

### DETAILED DESCRIPTION OF THE INVENTION

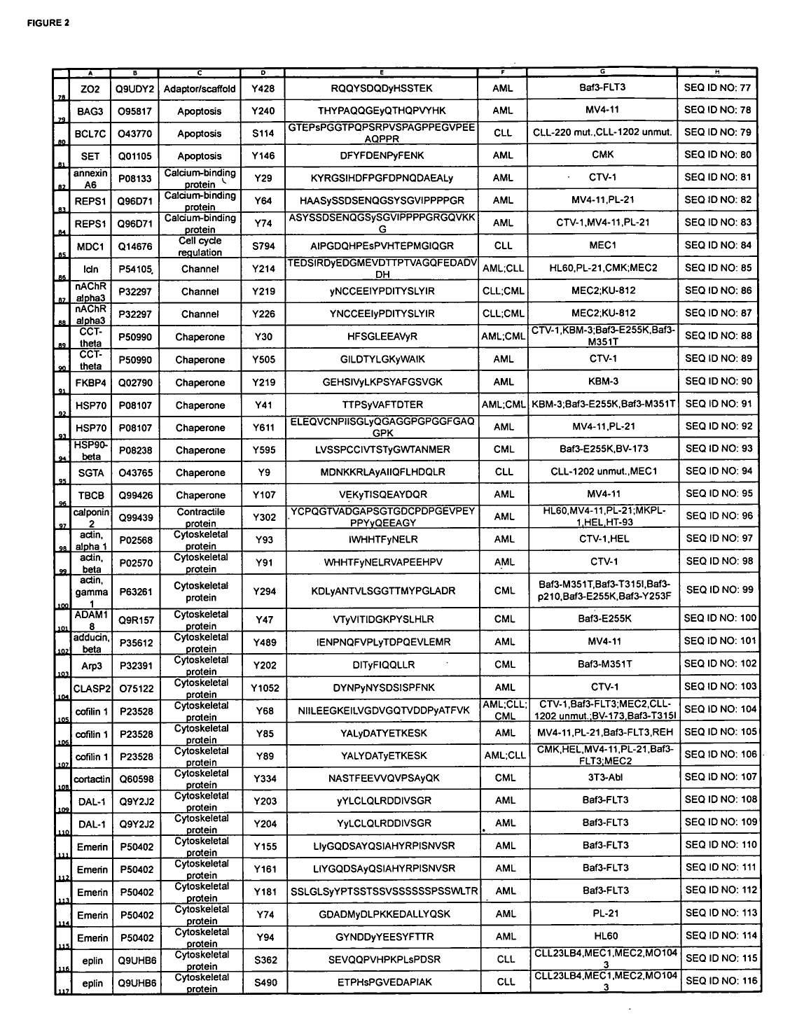

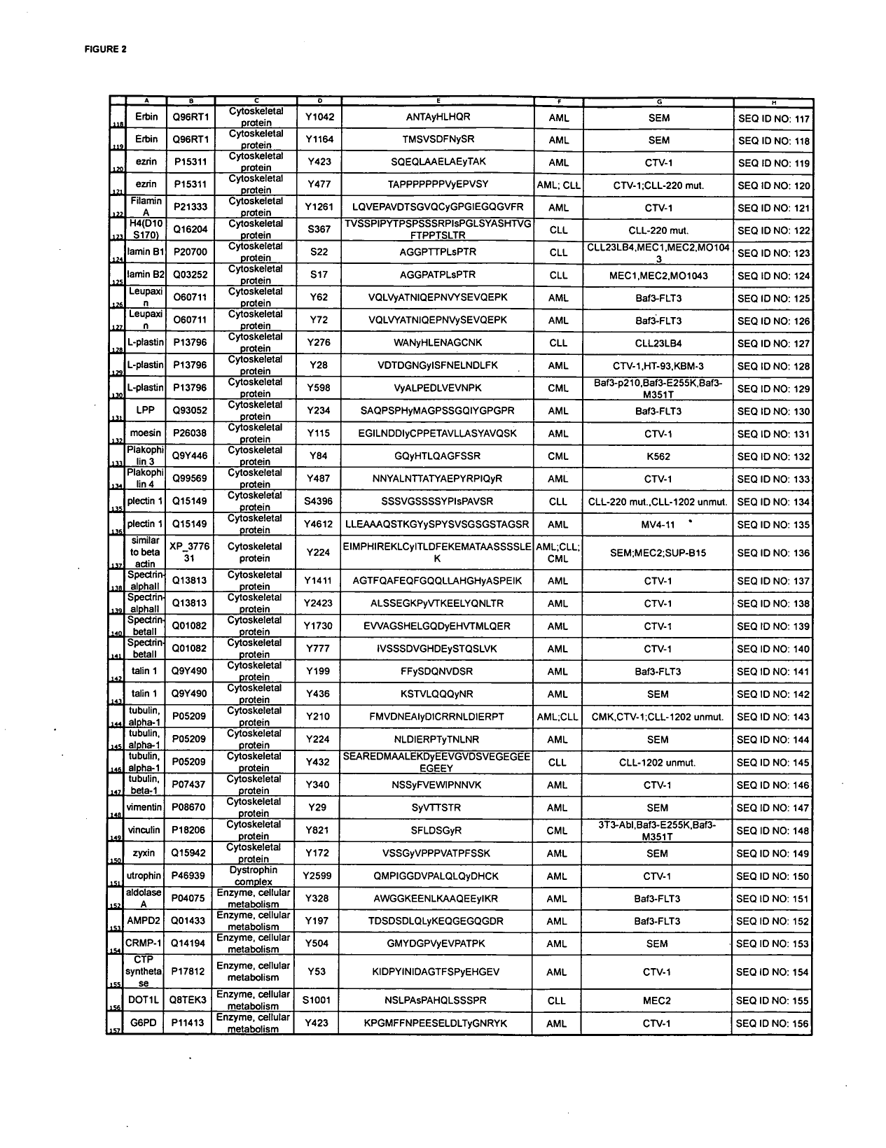

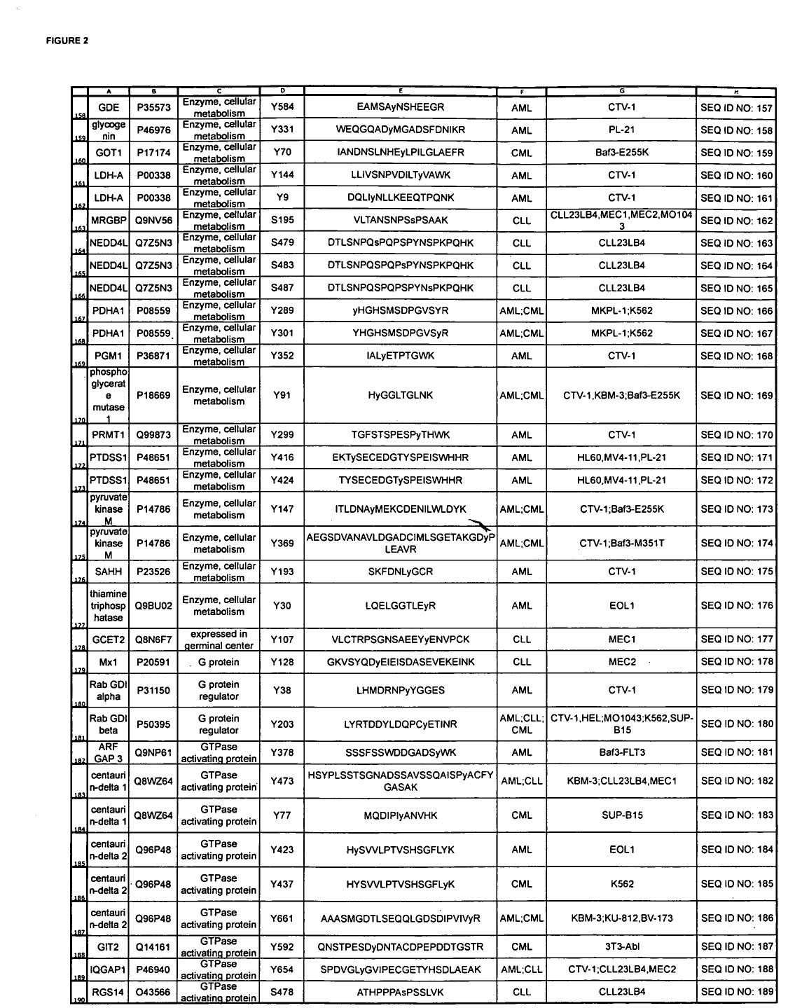

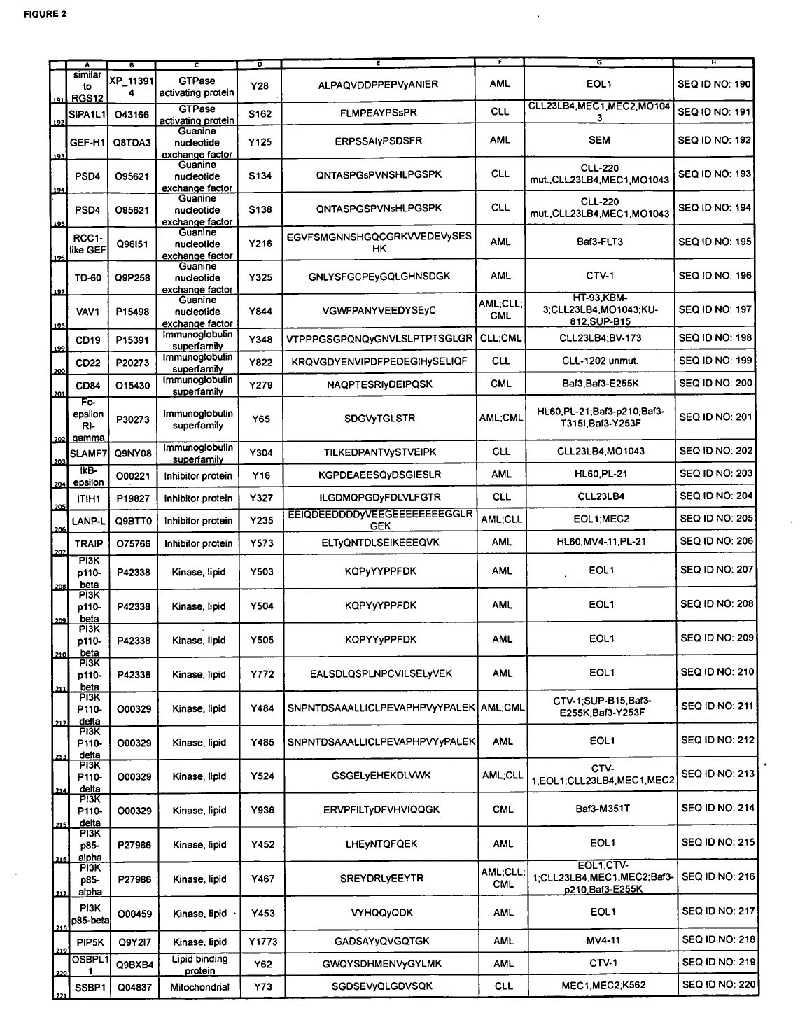

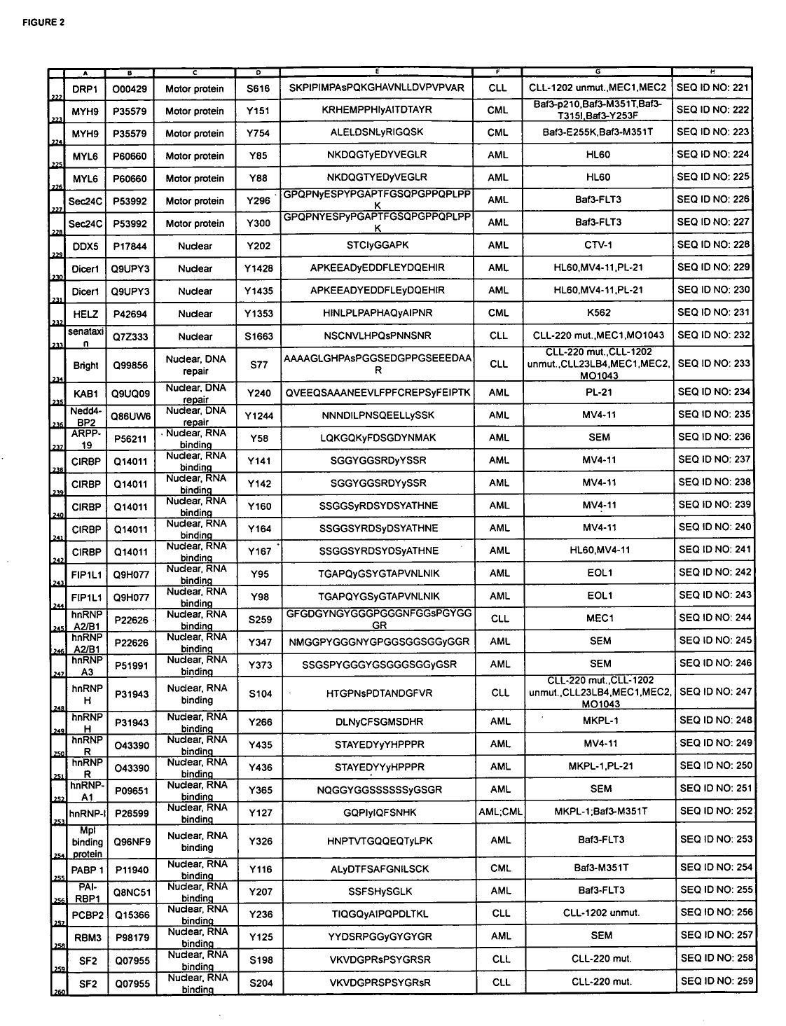

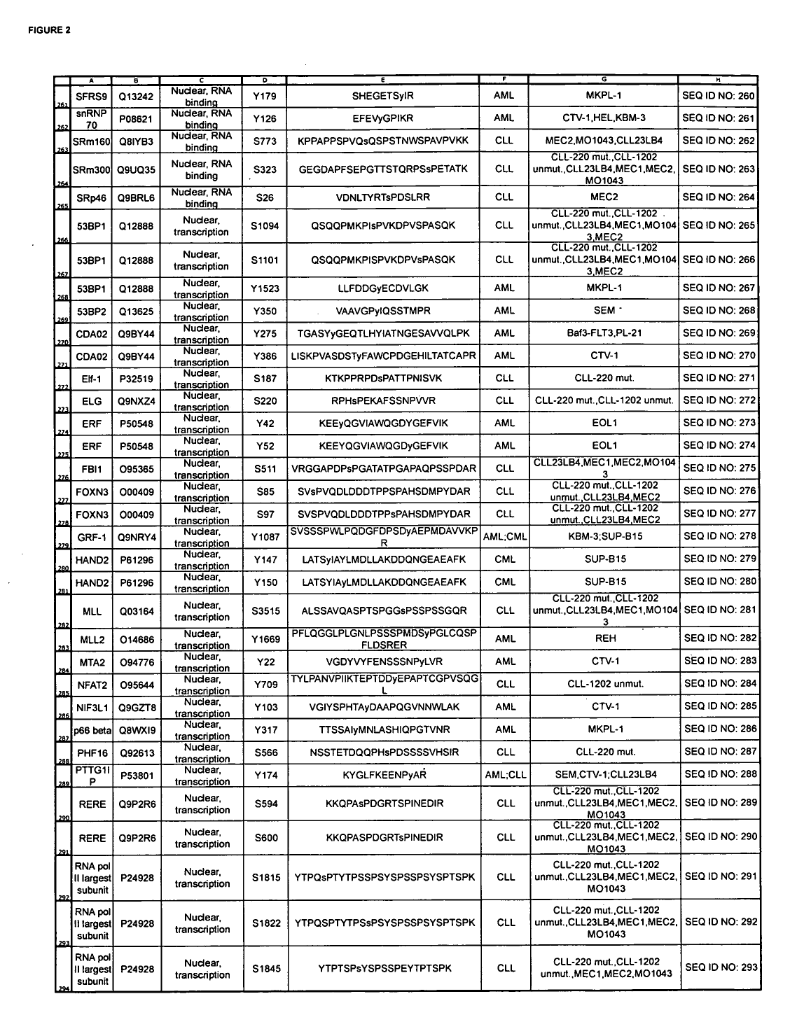

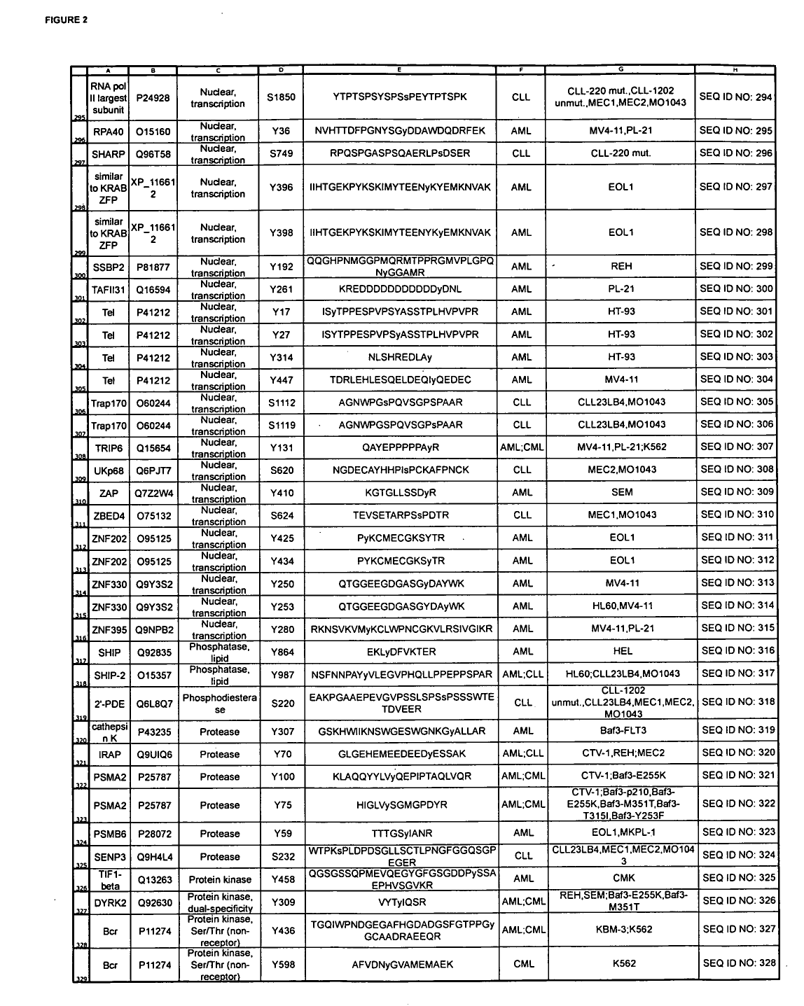

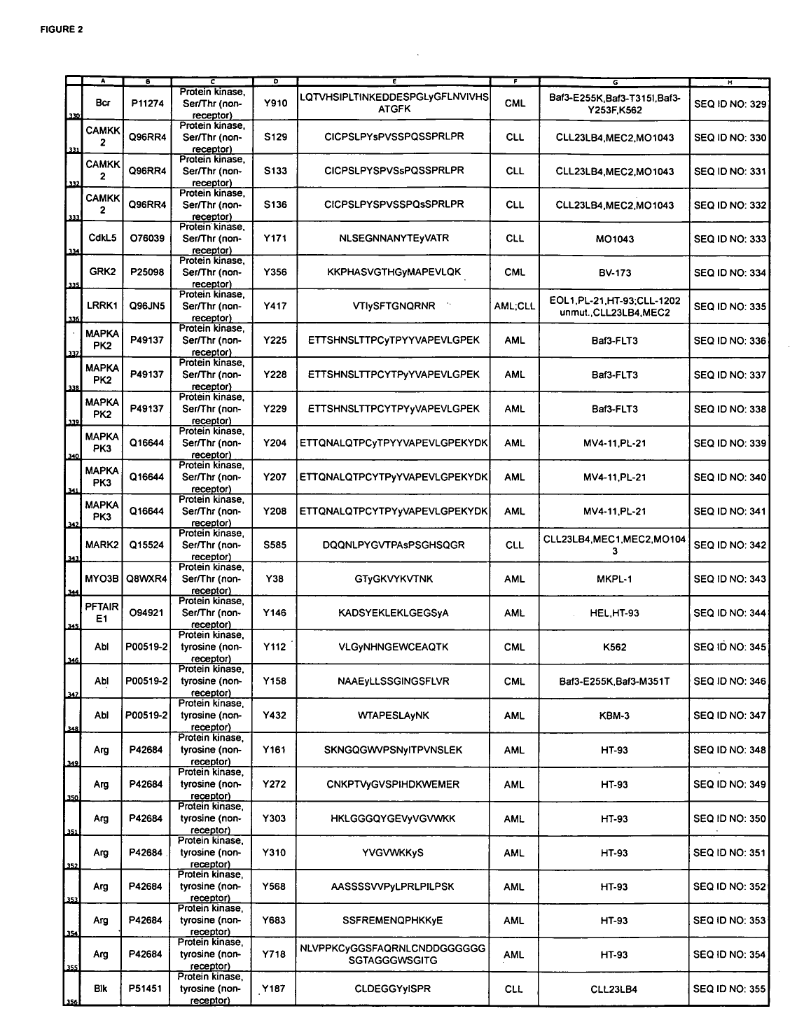

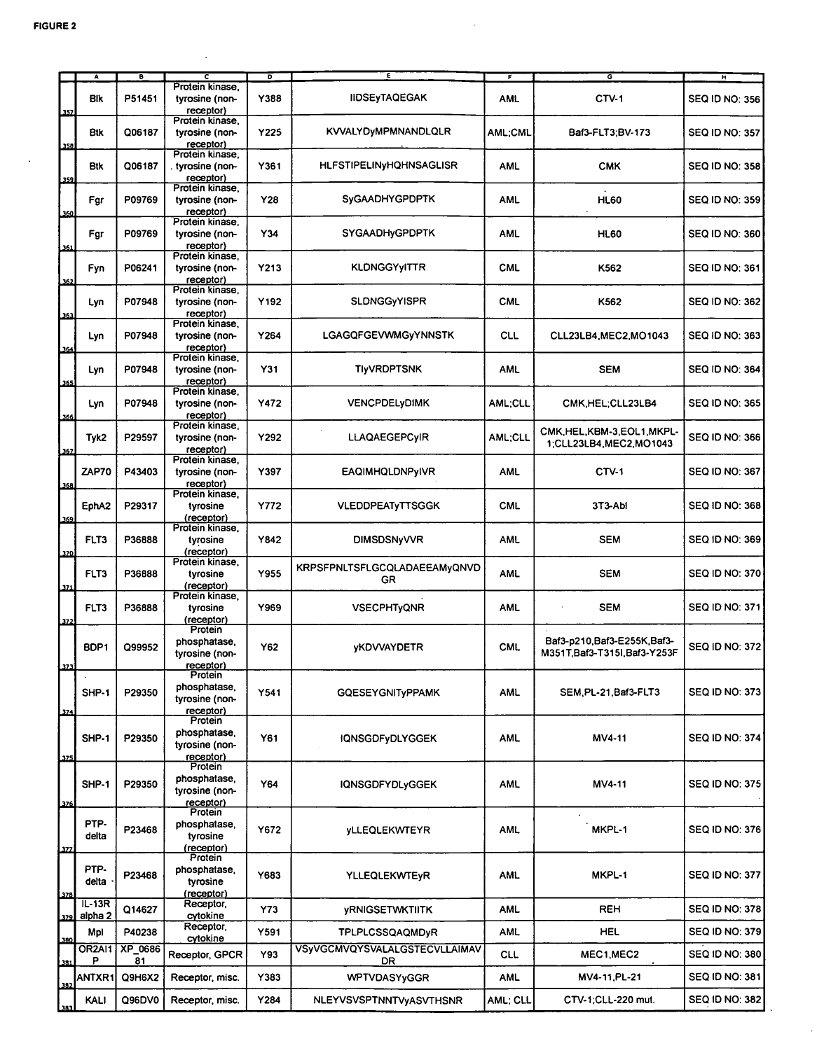

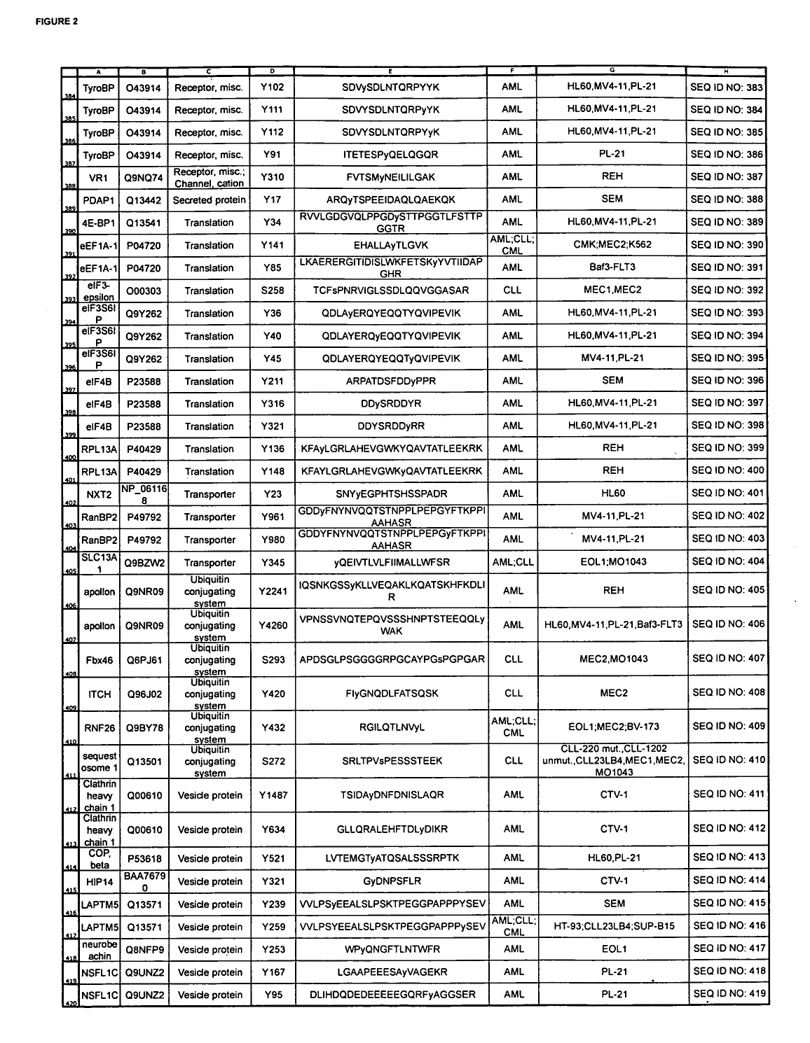

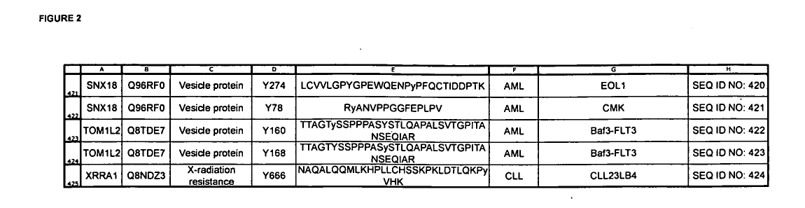

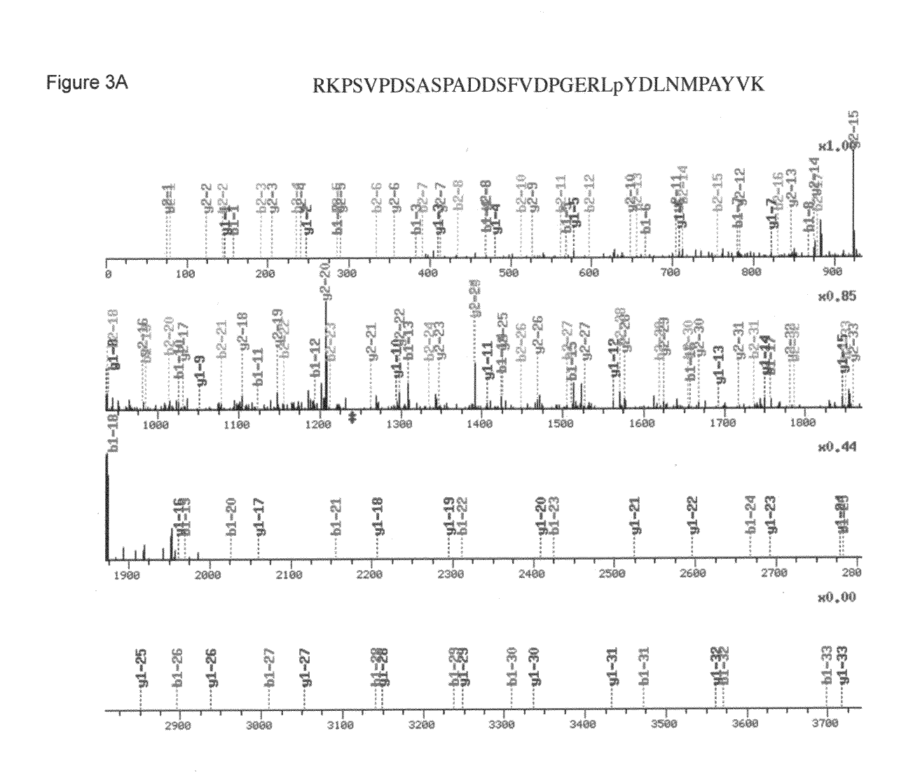

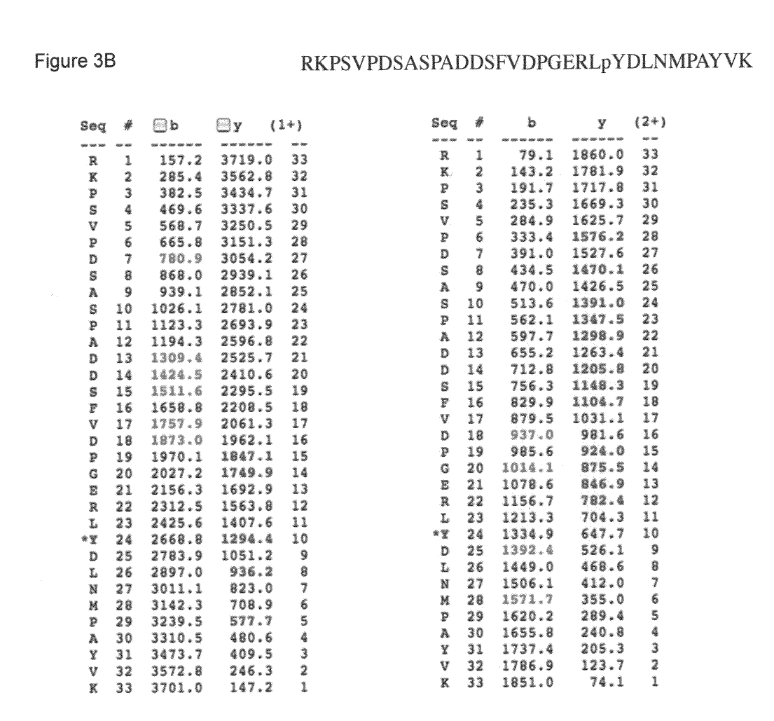

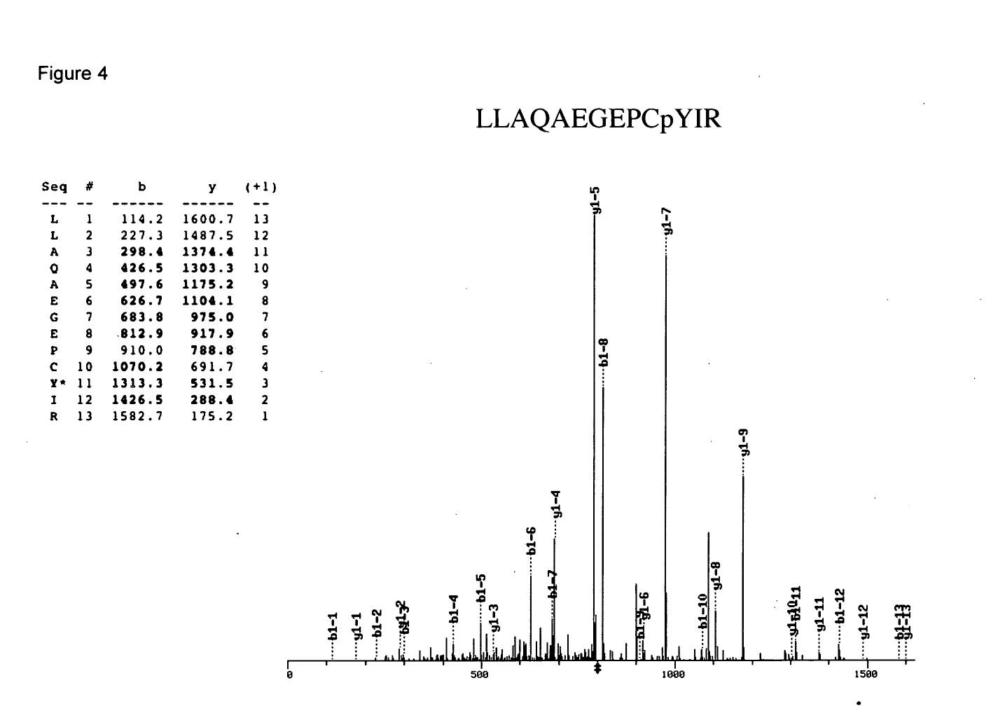

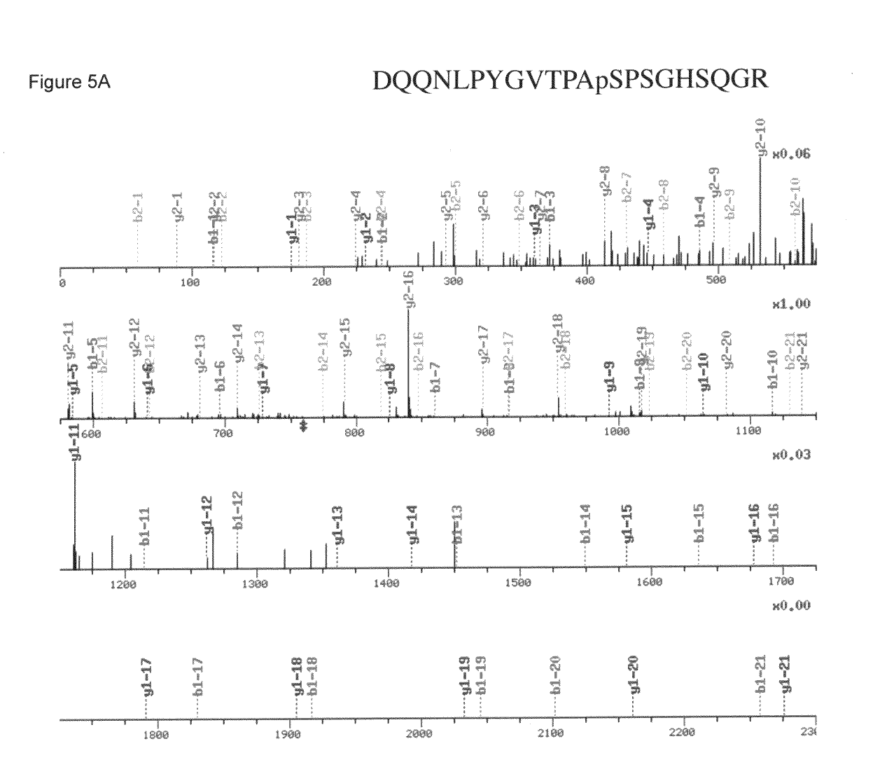

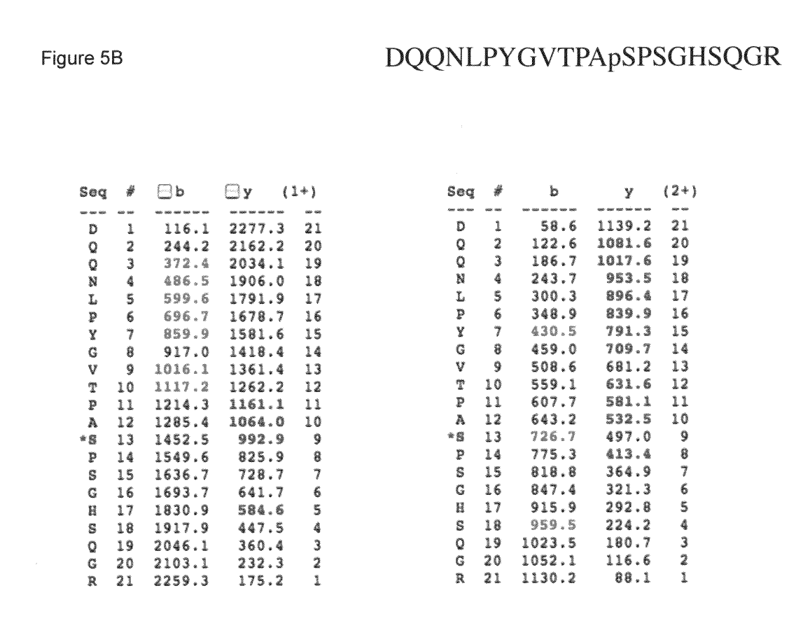

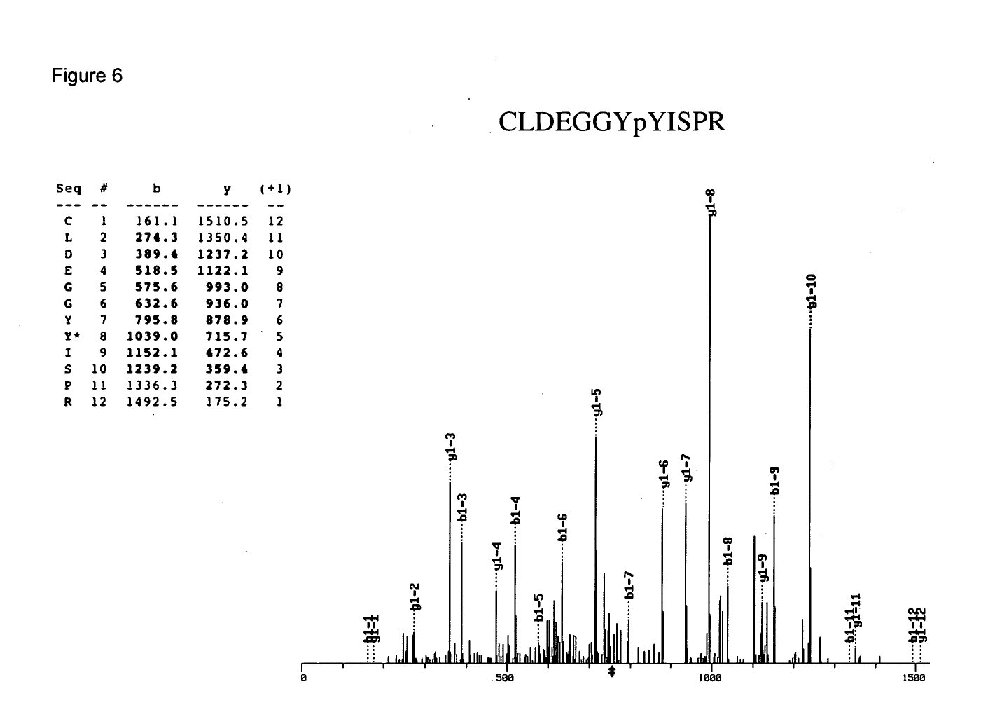

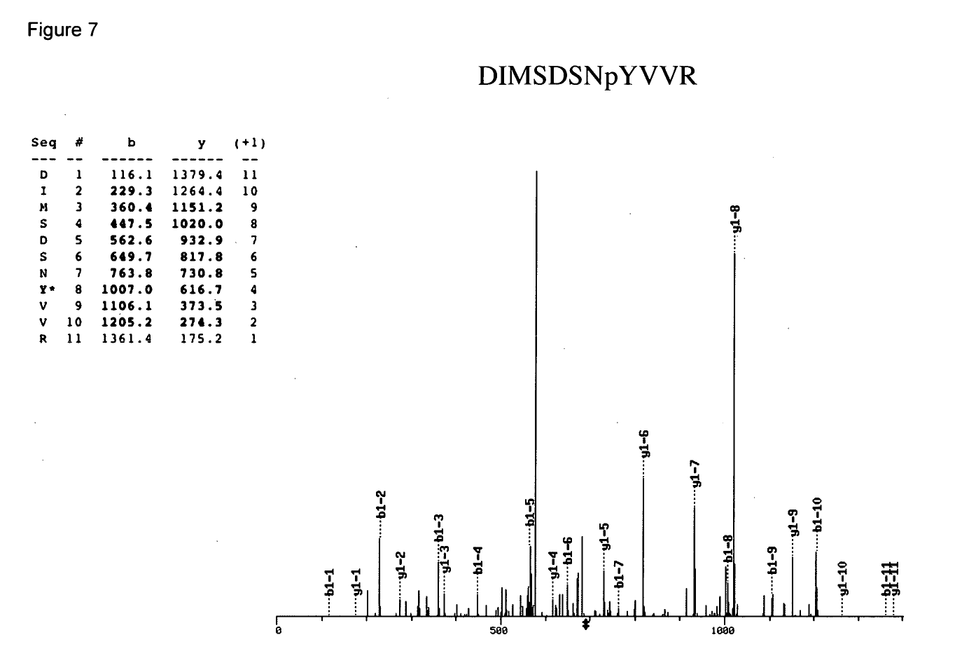

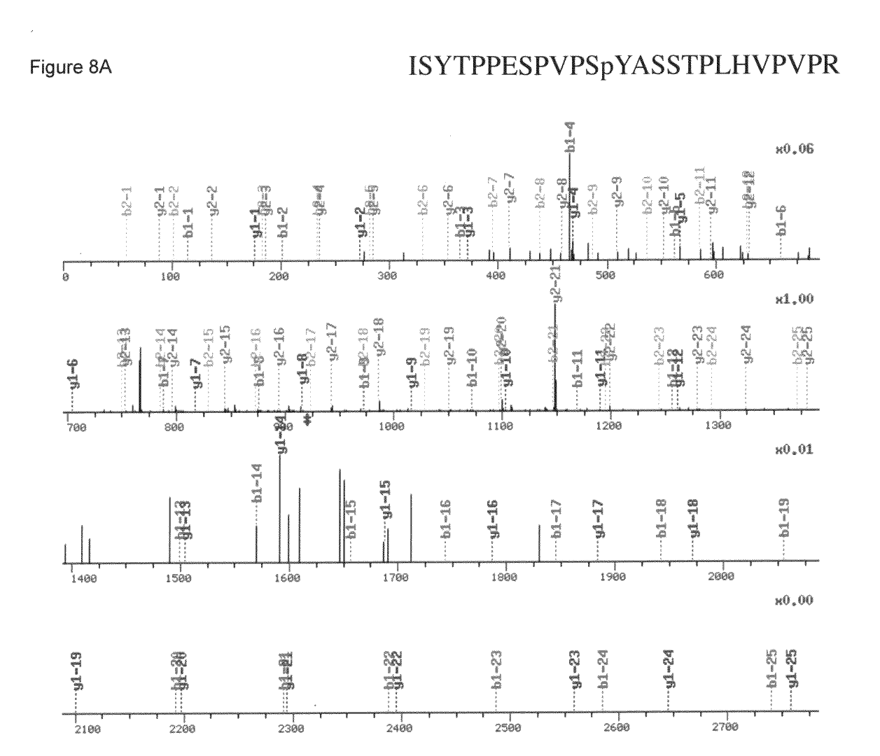

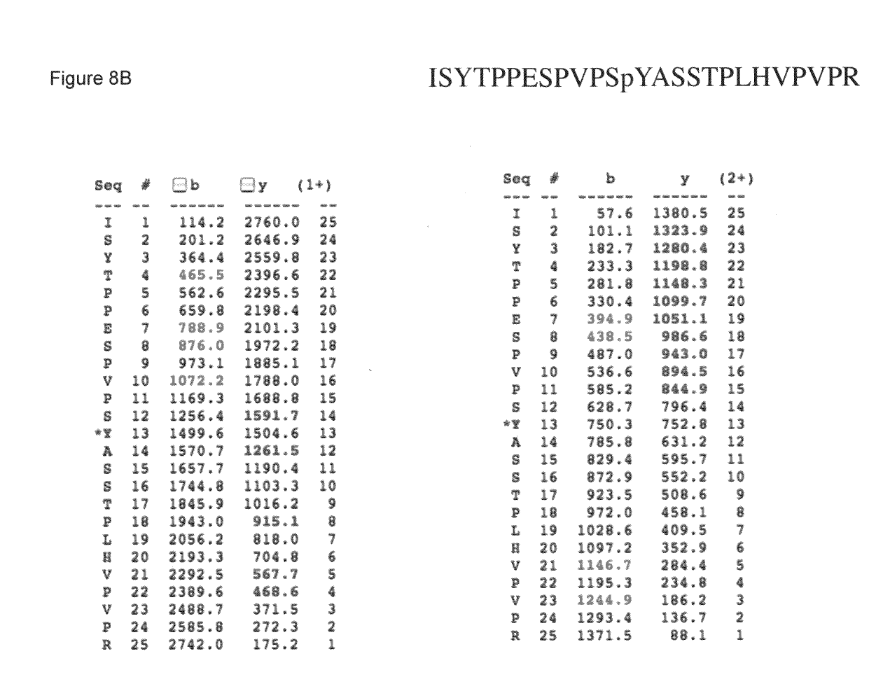

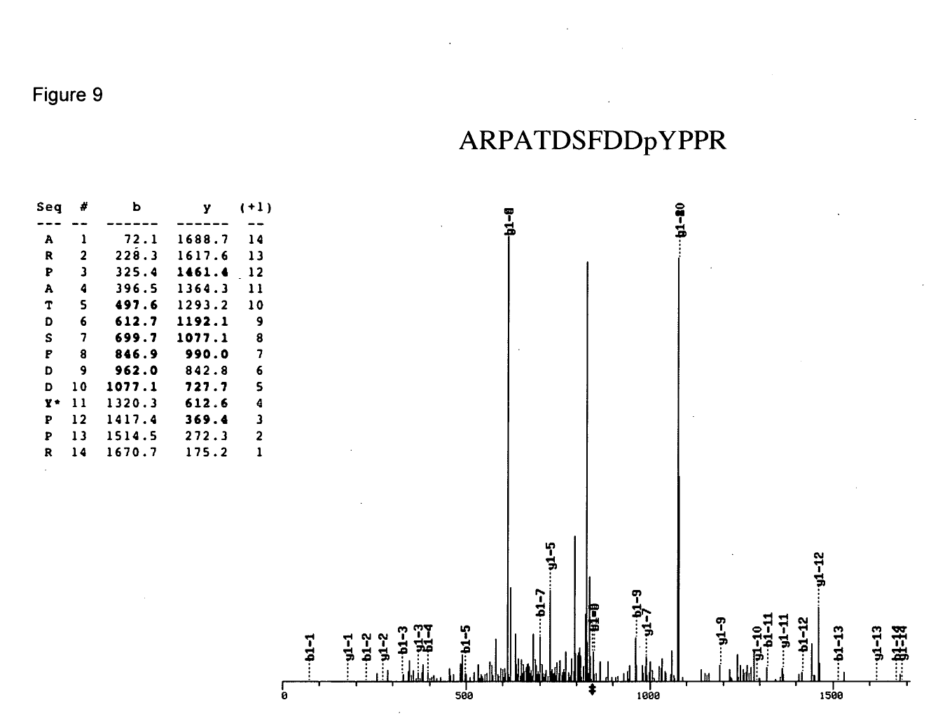

In accordance with the present invention, 424 novel protein phosphorylation sites in signaling proteins and pathways underlying huma Leukemia have now been discovered. These newly described phosphorylation sites were identified by employing the techniques described in “Immunoaffinity Isolation of Modified Peptides From Complex Mixtures,” U.S. Patent Publication No. 20030044848, Rush et al., using cellular extracts from a variety of leukemia-derived cell lines, e.g. HT-93, HEL, etc., as further described below. The novel phosphorylation sites (tyrosine or serine), and their corresponding parent proteins, disclosed herein are listed in Table 1. These phosphorylation sites correspond to numerous different parent proteins (the full sequences of which (human) are all publicly available in SwissProt database and their Accession numbers listed in Column B of Table 1/FIG. 2), each of which fall into discrete protein type groups, for example Adaptor/Scaffold proteins, Cytoskeletal proteins, Protein Kinases, and Vesicle proteins, etc. (see Column C of Table 1), the phosphorylation of which is relevant to signal transduction activity underlying Leukemias (AML, CML, CLL, and ALL), as disclosed herein.

The discovery of the 424 novel protein phosphorylation sites described herein enables the production, by standard methods, of new reagents, such as phosphorylation site-specific antibodies and AQUA peptides (heavy-isotope labeled peptides), capable of specifically detecting and/or quantifying these phosphorylated sites/proteins. Such reagents are highly useful, inter alia, for studying signal transduction events underlying the progression of Leukemia. Accordingly, the invention provides novel reagents—phospho-specific antibodies and AQUA peptides—for the specific detection and/or quantification of a Leukemia-related signaling protein/polypeptide only when phosphorylated (or only when not phosphorylated) at a particular phosphorylation site disclosed herein. The invention also provides methods of detecting and/or quantifying one or more phosphorylated Leukemia-related signaling proteins using the phosphorylation-site specific antibodies and AQUA peptides of the invention.

In part, the invention provides an isolated phosphorylation site-specific antibody that specifically binds a given Leukemia-related signaling protein only when phosphorylated (or not phosphorylated, respectively) at a particular tyrosine or serine enumerated in Column D of Table 1/FIG. 2 comprised within the phosphorylatable peptide site sequence enumerated in corresponding Column E. In further part, the invention provides a heavy-isotope labeled peptide (AQUA peptide) for the detection and quantification of a given Leukemia-related signaling protein, the labeled peptide comprising a particular phosphorylatable peptide site/sequence enumerated in Column E of Table 1/FIG. 2 herein. For example, among the reagents provided by the invention is an isolated phosphorylation site-specific antibody that specifically binds the Blk tyrosine kinase only when phosphorylated (or only when not phosphorylated) at tyrosine 187 (see Row 356 (and Columns D and E) of Table 1/FIG. 2). By way of further example, among the group of reagents provided by the invention is an AQUA peptide for the quantification of phosphorylated Blk tyrosine kinase, the AQUA peptide comprising the phosphorylatable peptide sequence listed in Column E, Row 356, of Table 1/FIG. 2 (which encompasses the phosphorylatable tyrosine at position 187).

In one embodiment, the invention provides an isolated phosphorylation site-specific antibody that specifically binds a huma Leukemia-related signaling protein selected from Column A of Table 1 (Rows 2-425) only when phosphorylated at the tyrosine or serine residue listed in corresponding Column D of Table 1, comprised within the phosphorylatable peptide sequence listed in corresponding Column E of Table 1 (SEQ ID NOs: 1-424), wherein said antibody does not bind said signaling protein when not phosphorylated at said tyrosine or serine. In another embodiment, the invention provides an isolated phosphorylation site-specific antibody that specifically binds a Leukemia-related signaling protein selected from Column A of Table 1 only when not phosphorylated at the tyrosine or serine residue listed in corresponding Column D of Table 1, comprised within the peptide sequence listed in corresponding Column E of Table 1 (SEQ ID NOs: 1-424), wherein said antibody does not bind said signaling protein when phosphorylated at said tyrosine. Such reagents enable the specific detection of phosphorylation (or non-phosphorylation) of a novel phosphorylatable site disclosed herein. The invention further provides immortalized cell lines producing such antibodies. In one preferred embodiment, the immortalized cell line is a rabbit or mouse hybridoma.

In another embodiment, the invention provides a heavy-isotope labeled peptide (AQUA peptide) for the quantification of a Leukemia-related signaling protein selected from Column A of Table 1, said labeled peptide comprising the phosphorylatable peptide sequence listed in corresponding Column E of Table 1 (SEQ ID NOs: 1-424), which sequence comprises the phosphorylatable tyrosine or serine listed in corresponding Column D of Table 1. In certain preferred embodiments, the phosphorylatable tyrosine or serine within the labeled peptide is phosphorylated, while in other preferred embodiments, the phosphorylatable residue within the labeled peptide is not phosphorylated.

Reagents (antibodies and AQUA peptides) provided by the invention may conveniently be grouped by the type of Leukemia-related signaling protein in which a given phosphorylation site (for which reagents are provided) occurs. The protein types for each respective protein (in which a phosphorylation site has been discovered) are provided in Column C of Table 1/FIG. 2, and include: Adaptor/Scaffold proteins, Apoptosis proteins, Calcium-binding proteins, Cell Cycle Regulation proteins, Channel proteins, Chaperone proteins, Contractile proteins, Cellular Metabolism enzymes, Cytoskeletal proteins, Dystrophin complex proteins, G protein and GTPase Activating proteins, Guanine Nucleotide Exchange Factors, Immunoglobulin Superfamily proteins, Inhibitor proteins, Lipid Kinases, Lipid Binding proteins, Lipid Phosphatases, Mitochondrial proteins, Motor proteins, Nuclear DNA Repair/RNA Binding/Transcription protein, Phosphodiesterases, Proteases, Serine/Threonine Protein Kinase, Tyrosine Kinases, Protein Phosphatases, Receptors, Secreted proteins, Translation/Transporter proteins, Ubiquitin Conjugating System proteins, Vesicle proteins, and X-Radiation Resistance proteins. Each of these distinct protein groups is considered a preferred subset of Leukemia-related signal transduction protein phosphorylation sites disclosed herein, and reagents for their detection/quantification may be considered a preferred subset of reagents provided by the invention.

Particularly preferred subsets of the phosphorylation sites (and their corresponding proteins) disclosed herein are those occurring on the following protein types/groups listed in Column C of Table 1/FIG. 2, Adaptor/Scaffold proteins, Cytoskeletal proteins, Cellular Metabolism enzymes, G Protein/GTPase Activating/Guanine Nucleotide Exchange Factor proteins, Immunoglobulin Superfamily proteins, Inhibitor proteins, Lipid Kinases, Nuclear DNA Repair/RNA Binding/Transcription proteins, Serine/Threonine Protein Kinases, Tyrosine Kinases, Protein Phosphatases, and Translation/Transporter proteins. Accordingly, among preferred subsets of reagents provided by the invention are isolated antibodies and AQUA peptides useful for the detection and/or quantification of the foregoing preferred protein/phosphorylation site subsets.

In one subset of preferred embodiments, there is provided:

(i) An isolated phosphorylation site-specific antibody that specifically binds an Adaptor/Scaffold protein selected from Column A, Rows 2-78, of Table 1 only when phosphorylated at the tyrosine or serine listed in corresponding Column D, Rows 2-78, of Table 1, comprised within the phosphorylatable peptide sequence listed in corresponding Column E, Rows 2-78, of Table 1 (SEQ ID NOs: 1-77), wherein said antibody does not bind said protein when not phosphorylated at said tyrosine or serine.
(ii) An equivalent antibody to (i) above that only binds the Adaptor/Scaffold protein when not phosphorylated at the disclosed site (and does not bind the protein when it is phosphorylated at the site).
(iii) A heavy-isotope labeled peptide (AQUA peptide) for the quantification of an Adaptor/Scaffold protein selected from Column A, Rows 2-78, said labeled peptide comprising the phosphorylatable peptide sequence listed in corresponding Column E, Rows 2-78, of Table 1 (SEQ ID NOs: 1-77), which sequence comprises the phosphorylatable tyrosine or serine listed in corresponding Column D, Rows 2-78, of Table 1.

Among this preferred subset of reagents, antibodies and AQUA peptides for the detection/quantification of the following Adaptor/Scaffold protein phosphorylation sites are particularly preferred: BCAP (Y392), Crk (Y251), and NCK1 (Y105) (see SEQ ID NOs: 7, 18, and 46).

In a second subset of preferred embodiments there is provided:

(i) An isolated phosphorylation site-specific antibody that specifically binds a Cytoskeletal protein selected from Column A, Rows 98-150, of Table 1 only when phosphorylated at the tyrosine or serine listed in corresponding Column D, Rows 98-150, of Table 1, comprised within the phosphorylatable peptide sequence listed in corresponding Column E, Rows 98-150, of Table 1 (SEQ ID NOs: 97-149), wherein said antibody does not bind said protein when not phosphorylated at said tyrosine or serine.
(ii) An equivalent antibody to (i) above that only binds the Cytoskeletal protein when not phosphorylated at the disclosed site (and does not bind the protein when it is phosphorylated at the site).
(iii) A heavy-isotope labeled peptide (AQUA peptide) for the quantification of a Leukemia-related signaling protein that is a Cytoskeletal protein selected from Column A, Rows 98-150, said labeled peptide comprising the phosphorylatable peptide sequence listed in corresponding Column E, Rows 98-150, of Table 1 (SEQ ID NOs: 97-149), which sequence comprises the phosphorylatable tyrosine or serine listed in corresponding Column D, Rows 98-150, of Table 1.

Among this preferred subset of reagents, antibodies and AQUA peptides for the detection/quantification of the following Cytoskeletal protein phosphorylation sites are particularly preferred: Ezrin (Y477) and Talin 1 (Y199) (see SEQ ID NOs: 120 and 141).

In another subset of preferred embodiments there is provided:

(i) An isolated phosphorylation site-specific antibody that specifically binds a Cellular Metabolism Enzyme selected from Column A, Rows 152-177, of Table 1 only when phosphorylated at the tyrosine or serine listed in corresponding Column D, Rows 152-177, of Table 1, comprised within the phosphorylatable peptide sequence listed in corresponding Column E, Rows 152-177, of Table 1 (SEQ ID NOs: 151-176), wherein said antibody does not bind said protein when not phosphorylated at said tyrosine or serine.
(ii) An equivalent antibody to (i) above that only binds the Cellular Metabolism Enzyme when not phosphorylated at the disclosed site (and does not bind the protein when it is phosphorylated at the site).
(iii) A heavy-isotope labeled peptide (AQUA peptide) for the quantification of a Leukemia-related signaling protein that is a Cellular Metabolism Enzyme selected from Column A, Rows 152-177, said labeled peptide comprising the phosphorylatable peptide sequence listed in corresponding Column E, Rows 152-177, of Table 1 (SEQ ID NOs: 151-176), which sequence comprises the phosphorylatable tyrosine or serine listed in corresponding Column D, Rows 152-177, of Table 1.

Among this preferred subset of reagents, antibodies and AQUA peptides for the detection/quantification of the following Cellular Metabolism Enzyme phosphorylation sites are particularly preferred: CRMP-1 (Y504) and NEDD4L (S479) (see SEQ ID NOs: 153 and 163).

In still another subset of preferred embodiments there is provided:

(i) An isolated phosphorylation site-specific antibody that specifically binds a G Protein/GTP Activating/Guanine Nucleotide Exchange Factor protein selected from Column A, Rows 179-198, of Table 1 only when phosphorylated at the tyrosine or serine listed in corresponding Column D, Rows 179-198, of Table 1, comprised within the phosphorylatable peptide sequence listed in corresponding Column E, Rows 179-198, of Table 1 (SEQ ID NOs: 178-197), wherein said antibody does not bind said protein when not phosphorylated at said tyrosine or serine.
(ii) An equivalent antibody to (i) above that only binds the G Protein/GTP Activating/Guanine Nucleotide Exchange Factor protein when not phosphorylated at the disclosed site (and does not bind the protein when it is phosphorylated at the site).
(iii) A heavy-isotope labeled peptide (AQUA peptide) for the quantification of a Leukemia-related signaling protein that is a G Protein/GTP Activating/Guanine Nucleotide Exchange Factor protein selected from Column A, Rows 179-198, said labeled peptide comprising the phosphorylatable peptide sequence listed in corresponding Column E, Rows 179-198, of Table 1 (SEQ ID NOs: 178-197), which sequence comprises the phosphorylatable tyrosine or serine listed in corresponding Column D, Rows 179-198, of Table 1.

Among this preferred subset of reagents, antibodies and AQUA peptides for the detection/quantification of the following G Protein/GTP Activating/Guanine Nucleotide Exchange Factor protein phosphorylation sites are particularly preferred: VAV1 (Tyr844) (see SEQ ID NO: 197).

In still another subset of preferred embodiments there is provided:

(i) An isolated phosphorylation site-specific antibody that specifically binds a Lipid Kinase selected from Column A, Rows 208-219, of Table 1 only when phosphorylated at the tyrosine listed in corresponding Column D, Rows 208-219, of Table 1, comprised within the phosphorylatable peptide sequence listed in corresponding Column E, Rows 208-219, of Table 1 (SEQ ID NOs: 207-218), wherein said antibody does not bind said protein when not phosphorylated at said tyrosine.
(ii) An equivalent antibody to (i) above that only binds the Lipid Kinase when not phosphorylated at the disclosed site (and does not bind the protein when it is phosphorylated at the site).
(iii) A heavy-isotope labeled peptide (AQUA peptide) for the quantification of a Leukemia-related signaling protein that is a Lipid Kinase selected from Column A, Rows 208-219, said labeled peptide comprising the phosphorylatable peptide sequence listed in corresponding Column E, Rows 208-219, of Table 1 (SEQ ID NOs: 207-218), which sequence comprises the phosphorylatable tyrosine or serine listed in corresponding Column D, Rows 208-219, of Table 1.

Among this preferred subset of reagents, antibodies and AQUA peptides for the detection/quantification of the following Lipid Kinase phosphorylation sites are particularly preferred: PI3K P110-delta (Y484) and PI3K p85-alpha (Y467) (see SEQ ID NOs: 211 and 216).

In still another subset of preferred embodiments there is provided:

(i) An isolated phosphorylation site-specific antibody that specifically binds a Nuclear/DNA Repair/RNA Binding/Transcription protein selected from Column A, Rows 229-316, of Table 1 only when phosphorylated at the tyrosine or serine listed in corresponding Column D, Rows 229-316, of Table 1, comprised within the phosphorylatable peptide sequence listed in corresponding Column E, Rows 229-316 of Table 1 (SEQ ID NOs: 228-315), wherein said antibody does not bind said protein when not phosphorylated at said tyrosine or serine.
(ii) An equivalent antibody to (i) above that only binds the Nuclear/DNA Repair/RNA Binding/Transcription protein when not phosphorylated at the disclosed site (and does not bind the protein when it is phosphorylated at the site).
(iii) A heavy-isotope labeled peptide (AQUA peptide) for the quantification of a Leukemia-related signaling protein that is a Nuclear/DNA Repair/RNA Binding/Transcription protein selected from Column A, Rows 229-316, said labeled peptide comprising the phosphorylatable peptide sequence listed in corresponding Column E, Rows 229-316, of Table 1 (SEQ ID NOs: 228-315), which sequence comprises the phosphorylatable tyrosine or serine listed in corresponding Column D, Rows 229-316, of Table 1.

Among this preferred subset of reagents, antibodies and AQUA peptides for the detection/quantification of the following Nuclear/DNA Repair/RNA Binding/Transcription protein phosphorylation sites are particularly preferred: 53BP1 (S1094), Elf-1 (S187), FOXN3 (S85), MLL (S3515), NFAT2 (Y709), and Tel (Y17) (see SEQ ID NOs: 265, 271, 276, 281, 284, and 301).

In yet another subset of preferred embodiments, there is provided:

(i) An isolated phosphorylation site-specific antibody that specifically binds a Serine/Threonine Protein Kinase selected from Column A, Rows 327-345, of Table 1 only when phosphorylated at the tyrosine or serine listed in corresponding Column D, Rows 327-345, of Table 1, comprised within the phosphorylatable peptide sequence listed in corresponding Column E, Rows 327-345, of Table 1 (SEQ ID NOs: 326-344), wherein said antibody does not bind said protein when not phosphorylated at said tyrosine or serine.
(ii) An equivalent antibody to (i) above that only binds the Serine/Threonine Protein Kinase when not phosphorylated at the disclosed site (and does not bind the protein when it is phosphorylated at the site).
(iii) A heavy-isotope labeled peptide (AQUA peptide) for the quantification of a Leukemia-related signaling protein that is a Serine/Threonine Protein Kinase selected from Column A, Rows 327-345, said labeled peptide comprising the phosphorylatable peptide sequence listed in corresponding Column E, Rows 327-345, of Table 1 (SEQ ID NOs: 326-344), which sequence comprises the phosphorylatable tyrosine or serine listed in corresponding Column D, Rows 327-345, of Table 1.

Among this preferred subset of reagents, antibodies and AQUA peptides for the detection/quantification of the following Serine/Threonine Protein Kinase phosphorylation sites are particularly preferred: Bcr (Y436, Y598, Y910), CAMKK2 (S129, S133, S136), CRK2 (Y356), LRKK1 (Y417), MARK2 (S585), MAPKAPK2 (Y225, Y228, Y229) and MAPKAPK3 (Y204, Y207, Y208) (see SEQ ID NOs: 327-332, and 334-342).

In yet another subset of preferred embodiments, there is provided:

(i) An isolated phosphorylation site-specific antibody specifically binds a Tyrosine Protein Kinase selected from Column A, Rows 346-372, of Table 1 only when phosphorylated at the tyrosine listed in corresponding Column D, Rows 346-372, of Table 1, comprised within the phosphorylatable peptide sequence listed in corresponding Column E, Rows 346-372, of Table 1 (SEQ ID NOs: 345-371), wherein said antibody does not bind said protein when not phosphorylated at said tyrosine.
(ii) An equivalent antibody to (i) above that only binds the Tyrosine Protein Kinase when not phosphorylated at the disclosed site (and does not bind the protein when it is phosphorylated at the site).
(iii) A heavy-isotope labeled peptide (AQUA peptide) for the quantification of a Leukemia-related signaling protein that is a Tyrosine Protein Kinase selected from Column A, Rows 346-372, said labeled peptide comprising the phosphorylatable peptide sequence listed in corresponding Column E, Rows 346-372, of Table 1 (SEQ ID NOs: 345-371), which sequence comprises the phosphorylatable tyrosine listed in corresponding Column D, Rows 346-372, of Table 1.

Among this preferred subset of reagents, antibodies and AQUA peptides for the detection/quantification of the following Tyrosine Protein Kinase phosphorylation sites are particularly preferred: Arg (Y161, 272, Y303, Y310, Y568, Y683, Y718), Blk (Y187, Y388), Lyn (Y192, Y264, Y31, Y472), Tyk2 (Y292), and FLT3 (Y842, Y955, Y969) (see SEQ ID NOs: 348-356, 362-366, and 369-371).

In yet another subset of preferred embodiments, there is provided:

(i) An isolated phosphorylation site-specific antibody that specifically binds a Protein Phosphatase selected from Column A, Rows 373-378, of Table 1 only when phosphorylated at the tyrosine listed in corresponding Column D, Rows 373-378, of Table 1, comprised within the phosphorylatable peptide sequence listed in corresponding Column E, Rows 373-378, of Table 1 (SEQ ID NOs: 372-377), wherein said antibody does not bind said protein when not phosphorylated at said tyrosine.
(ii) An equivalent antibody to (i) above that only binds the Protein Phosphatase when not phosphorylated at the disclosed site (and does not bind the protein when it is phosphorylated at the site).
(iii) A heavy-isotope labeled peptide (AQUA peptide) for the quantification of a Leukemia-related signaling protein that is a Protein Phosphatase selected from Column A, Rows 373-378, said labeled peptide comprising the phosphorylatable peptide sequence listed in corresponding Column E, Rows 373-378, of Table 1 (SEQ ID NOs: 372-377), which sequence comprises the phosphorylatable tyrosine listed in corresponding Column D, Rows 373-378, of Table 1.

Among this preferred subset of reagents, antibodies and AQUA peptides for the detection/quantification of the following Protein Phosphatase phosphorylation sites are particularly preferred: SHP-1 (Y541, Y61, Y64) (see SEQ ID NO: 373-375).

In still another subset of preferred embodiments, there is provided:

(i) An isolated phosphorylation site-specific antibody that specifically binds a Translation/Transporter protein selected from Column A, Rows 390-405, of Table 1 only when phosphorylated at the tyrosine or serine listed in corresponding Column D, Rows 390405, of Table 1, comprised within the phosphorylatable peptide sequence listed in corresponding Column E, Rows 390-405, of Table 1 (SEQ ID NOs: 389-404), wherein said antibody does not bind said protein when not phosphorylated at said tyrosine or serine.
(ii) An equivalent antibody to (i) above that only binds the Translation/Transporter protein when not phosphorylated at the disclosed site (and does not bind the protein when it is phosphorylated at the site).
(iii) A heavy-isotope labeled peptide (AQUA peptide) for the quantification of a Leukemia-related signaling protein that Translation/Transporter protein selected from Column A, Rows 390-405, said labeled peptide comprising the phosphorylatable peptide sequence listed in corresponding Column E, Rows 390-405, of Table 1 (SEQ ID NOs: 389-404), which sequence comprises the phosphorylatable tyrosine or serine listed in corresponding Column D, Rows 390-405, of Table 1.

Among this preferred subset of reagents, antibodies and AQUA peptides for the detection/quantification of the following Translation/Transporter protein phosphorylation sites are particularly preferred: eIF4B (Y211, Y316, Y321) (see SEQ ID NOs: 396-398).

In still another subset of preferred embodiments, there is provided:

(i) An isolated phosphorylation site-specific antibody that specifically binds an Immunoglobulin Superfamily protein selected from Column A, Rows 199-203, of Table 1 only when phosphorylated at the tyrosine listed in corresponding Column D, Rows 199-203, of Table 1, comprised within the phosphorylatable peptide sequence listed in corresponding Column E, Rows 199-203, of Table 1 (SEQ ID NOs: 198-202), wherein said antibody does not bind said protein when not phosphorylated at said tyrosine.
(ii) An equivalent antibody to (i) above that only binds the Immunoglobulin Superfamily protein when not phosphorylated at the disclosed site (and does not bind the protein when it is phosphorylated at the site).
(iii) A heavy-isotope labeled peptide (AQUA peptide) for the quantification of a Leukemia-related signaling protein that is an Immunoglobulin Superfamily protein selected from Column A, Rows 199-203, said labeled peptide comprising the phosphorylatable peptide sequence listed in corresponding Column E, Rows 199-203, of Table 1 (SEQ ID NOs: 198-202), which sequence comprises the phosphorylatable tyrosine listed in corresponding Column D, Rows 199-203, of Table 1.

In still another subset of preferred embodiments, there is provided:

(i) An isolated phosphorylation site-specific antibody that specifically binds an Inhibitor protein selected from Column A, Rows 204-207, of Table 1 only when phosphorylated at the tyrosine listed in corresponding Column D, Rows 204-207, of Table 1, comprised within the phosphorylatable peptide sequence listed in corresponding Column E, Rows 204-207, of Table 1 (SEQ ID NOs: 203-206), wherein said antibody does not bind said protein when not phosphorylated at said tyrosine.
(ii) An equivalent antibody to (i) above that only binds the Inhibitor protein when not phosphorylated at the disclosed site (and does not bind the protein when it is phosphorylated at the site).
(iii) A heavy-isotope labeled peptide (AQUA peptide) for the quantification of a Leukemia-related signaling protein that is an Inhibitor protein selected from Column A, Rows 204-207, said labeled peptide comprising the phosphorylatable peptide sequence listed in corresponding Column E, Rows 204-207, of Table 1 (SEQ ID NOs: 203-206), which sequence comprises the phosphorylatable tyrosine listed in corresponding Column D, Rows 204-207, of Table 1.

The invention also provides, in part, an immortalized cell line producing an antibody of the invention, for example, a cell line producing an antibody within any of the foregoing preferred subsets of antibodies. In one preferred embodiment, the immortalized cell line is a rabbit hybridoma or a mouse hybridoma.

In certain other preferred embodiments, a heavy-isotope labeled peptide (AQUA peptide) of the invention (for example, an AQUA peptide within any of the foregoing preferred subsets of AQUA peptides) comprises a disclosed site sequence wherein the phosphorylatable tyrosine or serine is phosphorylated. In certain other preferred embodiments, a heavy-isotope labeled peptide of the invention comprises a disclosed site sequence wherein the phosphorylatable tyrosine or serine is not phosphorylated.

The foregoing subsets of preferred reagents of the invention should not be construed as limiting the scope of the invention, which, as noted above, includes reagents for the detection and/or quantification of disclosed phosphorylation sites on any of the other protein type/group subsets (each a preferred subset) listed in Column C of Table 1/FIG. 2.

Also provided by the invention are methods for detecting or quantifying a Leukemia-related signaling protein that is tyrosine- or serine-phosphorylated, said method comprising the step of utilizing one or more of the above-described reagents of the invention to detect or quantify one or more Leukemia-related signaling protein(s) selected from Column A of Table 1 only when phosphorylated at the tyrosine or serine listed in corresponding Column D of Table 1. In certain preferred embodiments of the methods of the invention, the reagents comprise a subset of preferred reagents as described above.

The identification of the disclosed novel Leukemia-related signaling protein phosphorylation sites, and the standard production and use of the reagents provided by the invention are described in further detail below and in the Examples that follow.

All cited references are hereby incorporated herein, in their entirety, by reference. The Examples are provided to further illustrate the invention, and do not in any way limit its scope, except as provided in the claims appended hereto.

TABLE 1
Newly Discovered Leukemia-related Phosphorylation Sites.

| | Protein | Accession | | Phospho- | | |
|---|---|---|---|---|---|---|
| 1 | Name | No | Protein Type | Residue | Phosphorylation Site Sequence | SEQ ID NO: |
| 2 | Abi-1 | O76049 | Adaptor/scaffold | Y198 | NTPyKTLEPVKPPTVPNDYMTSPAR | SEQ ID NO: 1 |
| 3 | Abi-1 | O76049 | Adaptor/scaffold | Y213 | NTPYKTLEPVKPPTVPNDyMTSPAR | SEQ ID NO: 2 |
| 4 | Abi-1 | O76049 | Adaptor/scaffold | Y23 | ALIESyQNLTR | SEQ ID NO: 3 |
| 5 | Abi-2 | Q9NYB9 | Adaptor/scaffold | Y213 | TLEPVRPPVVPNDyVPSPTR | SEQ ID NO: 4 |
| 6 | AKAP2 | Q9Y2D5 | Adaptor/scaffold | S383 | DALGDSLQVPVsPSSTTSSR | SEQ ID NO: 5 |
| 7 | ankyrin 1 | P16157 | Adaptor/scaffold | Y215 | TGFTPLHIAAHyENLNVAQLLLNR | SEQ ID NO: 6 |
| 8 | BCAP | Q8NAC8 | Adaptor/scaffold | Y392 | SQERPGNFyVSSESIR | SEQ ID NO: 7 |
| 9 | BCAP | Q8NAC8 | Adaptor/scaffold | Y516 | HSQHLPAKVEFGVyESGPR | SEQ ID NO: 8 |
| 10 | BIN1 | O00499 | Adaptor/scaffold | S331 | VNHEPEPAGGATPGATLPKsPSQLR | SEQ ID NO: 9 |
| 11 | CASKIN2 | Q8WXE0 | Adaptor/scaffold | Y253 | NTyNQTALDIVNQFTTSQASR | SEQ ID NO: 10 |
| 12 | Cas-L | Q14511 | Adaptor/scaffold | Y106 | YQVPNPQAAPRDTIyQVPPSYQNQGIYQVPT | SEQ ID NO: 11 |
| 13 | Cas-L | Q14511 | Adaptor/scaffold | Y118 | YQVPNPQAAPRDTIYQVPPSYQNQGIyQVPT | SEQ ID NO: 12 |
| 14 | Cas-L | Q14511 | Adaptor/scaffold | Y214 | GPVFSVPVGEIKPQGVyDIPPTK | SEQ ID NO: 13 |
| 15 | Cas-L | Q14511 | Adaptor/scaffold | Y317 | HQSLSPNHPPPQLGQSVGSQNDAyDVPR | SEQ ID NO: 14 |
| 16 | Cas-L | Q14511 | Adaptor/scaffold | Y345 | ANPQERDGVyDVPLHNPPDAK | SEQ ID NO: 15 |
| 17 | CbI | P22681 | Adaptor/scaffold | Y552 | DLPPPPPPDRPySVGAESRPQR | SEQ ID NO: 16 |
| 18 | CD2AP | Q9Y5K6 | Adaptor/scaffold | Y548 | DTCYSPKPSVyLSTPSSASK | SEQ ID NO: 17 |
| 19 | Crk | P46108 | Adaptor/scaffold | Y251 | RVPNAyDKTALALEVGELVK | SEQ ID NO: 18 |
| 20 | diaphanous | O60610 | Adaptor/scaffold | Y365 | VQLNVFDEQGEEDSyDLKGR | SEQ ID NO: 19 |
| | 1 | | | | | |
| 21 | DNMBP | Q9Y2L3 | Adaptor/scaffold | Y1215 | HPEIVGySVPGR | SEQ ID NO: 20 |
| 22 | Dok2 | O60496 | Adaptor/scaffold | Y139 | QSRPCMEENELySSAVTVGPHK | SEQ ID NO: 21 |
| 23 | Dok2 | O60496 | Adaptor/scaffold | Y402 | GWQPGTEyDNVVLKKGPK | SEQ ID NO: 22 |
| 24 | Dok3 | Q9H666 | Adaptor/scaffold | Y208 | RGLVPMEENSIySSWQEVGEFPVVVQR | SEQ ID NO: 23 |
| 25 | Dok3 | Q9H666 | Adaptor/scaffold | Y381 | KMHLAEPGPQSLPLLLGPEPNDLASGLyASVCKR | SEQ ID NO: 24 |
| 26 | Dok3 | Q9H666 | Adaptor/scaffold | Y398 | ASGPPGNEHLyENLCVLEASPTLHGGEPEPHEGPGSR | SEQ ID NO: 25 |
| 27 | Dok3 | Q9H666 | Adaptor/scaffold | Y432 | SPTTSPIyHNGQDLSWPGPANDSTLEAQYR | SEQ ID NO: 26 |
| 28 | Dok3 | Q9H666 | Adaptor/scaffold | Y453 | SPTTSPIYHNGQDLSWPGPANDSTLEAQyRR | SEQ ID NO: 27 |
| 29 | EPS15R | Q9UBC2 | Adaptor/scaffold | Y74 | KIWDLADPEGKGFLDKQGFy | SEQ ID NO: 28 |
| 30 | FCHSD2 | O94868 | Adaptor/scaffold | S687 | SSLYFPRsPSANEK | SEQ ID NO: 29 |
| 31 | Frigg | Q9UH99 | Adaptor/scaffold | Y140 | KATEDFLGSSSGYSSEDDyVGYSDVDQQSSSSR | SEQ ID NO: 30 |
| 32 | Frigg | Q9UH99 | Adaptor/scaffold | Y143 | KATEDFLGSSSGYSSEDDYVGySDVDQQSSSSR | SEQ ID NO: 31 |
| 33 | G3BP-1 | Q13283 | Adaptor/scaffold | Y56 | NSSYVHGGLDSNGKPADAVyGQK | SEQ ID NO: 32 |
| 34 | Gab1 | Q13480 | Adaptor/scaffold | Y242 | HGMNGFFQQQMIyDSPPSRAPSASVDSSLYNLPR | SEQ ID NO: 33 |
| 35 | Gab1 | Q13480 | Adaptor/scaffold | Y317 | HVSISYDIPPTPGNTyQIPR | SEQ ID NO: 34 |
| 36 | Gab2 | Q9UQC2 | Adaptor/scaffold | Y249 | LAQGNGHCVNGISGQVHGFySLPKPSR | SEQ ID NO: 35 |
| 37 | Gab2 | Q9UQC2 | Adaptor/scaffold | Y293 | GSLTGSETDNEDVyTFK | SEQ ID NO: 36 |
| 38 | Gab2 | Q9UQC2 | Adaptor/scaffold | Y324 | EFGDLLVDNMDVPATPLSAyQIPR | SEQ ID NO: 37 |
| 39 | HS1 | P14317 | Adaptor/scaffold | Y140 | SAVGFDyKGEVEKHTSQK | SEQ ID NO: 38 |
| 40 | Inter- | Q9NZM3 | Adaptor/scaffold | Y552 | LIyLVPEK | SEQ ID NO: 39 |
| | sectin 2 | | | | | |
| 41 | Inter- | Q9NZM3 | Adaptor/scaffold | Y979 | AVNKKPTSAAyS | SEQ ID NO: 40 |
| | sectin 2 | | | | | |
| 42 | IRS-2 | Q9Y4H2 | Adaptor/scaffold | Y632 | VAYHPYPEDyGDIEIGSHR | SEQ ID NO: 41 |
| 43 | LAB | Q9GZY6 | Adaptor/scaffold | Y110 | HGSEEAyIDPIAMEYYNWGR | SEQ ID NO: 42 |
| 44 | LAB | Q9GZY6 | Adaptor/scaffold | Y118 | HGSEEAYIDPIAMEyYNWGR | SEQ ID NO: 43 |
| 45 | LAB | Q9GZY6 | Adaptor/scaffold | Y119 | HGSEEAYIDPIAMEYyNWGR | SEQ ID NO: 44 |
| 46 | LAB | Q9GZY6 | Adaptor/scaffold | Y58 | QENAQSSAAAQTySLAR | SEQ ID NO: 45 |
| 47 | NCK1 | P16333 | Adaptor/scaffold | Y105 | RKPSVPDSASPADDSFVDPGERLyDLNMPAYVK | SEQ ID NO: 46 |
| 48 | NCK1 | P16333 | Adaptor/scaffold | Y268 | NYVTVMQNNPLTSGLEPSPPQCDyIRPSLTGK | SEQ ID NO: 47 |
| 49 | NCK2 | O43639 | Adaptor/scaffold | Y110 | DASPTPSTDAEYPANGSGADRIyDLNIPAFVK | SEQ ID NO: 48 |
| 50 | NCK2 | O43639 | Adaptor/scaffold | Y99 | DASPTPSTDAEyPANGSGADRIYDLNIPAFVK | SEQ ID NO: 49 |
| 51 | NCKIPSD | Q9NZQ3 | Adaptor/scaffold | Y161 | QHSLPSSEHLGADGGLyQIPPQPR | SEQ ID NO: 50 |
| 52 | PAG | Q9NYK0 | Adaptor/scaffold | Y163 | SVDGDQGLGMEGPyEVLK | SEQ ID NO: 51 |
| 53 | PAG | Q9NYK0 | Adaptor/scaffold | Y181 | DSSSQENMVEDCLyETVK | SEQ ID NO: 52 |
| 54 | PAG | Q9NYK0 | Adaptor/scaffold | Y341 | NKSGQSLTVPESTyTSIQGDPQRSPS | SEQ ID NO: 53 |
| 55 | PAG | Q9NYK0 | Adaptor/scaffold | Y359 | SGQSLTVPESTYTSIQGDPQRSPSSCNDLyATVK | SEQ ID NO: 54 |
| 56 | PAG | Q9NYK0 | Adaptor/scaffold | Y417 | ATLGTNGHHGLVPKENDyESISDLQQGR | SEQ ID NO: 55 |
| 57 | PARD3 | Q8TEW0 | Adaptor/scaffold | Y388 | FSPDSQyIDNR | SEQ ID NO: 56 |
| 58 | PSTPIP2 | Q9H939 | Adaptor/scaffold | Y322 | RIPDDPDySVVEDYSLLYQ | SEQ ID NO: 57 |
| 59 | PSTPIP2 | Q9H939 | Adaptor/scaffold | Y332 | RIPDDPDYSVVEDYSLLyQ | SEQ ID NO: 58 |
| 60 | RA70 | Q9UED8 | Adaptor/scaffold | Y237 | FILQDLGSDVIPEDDEERGELyDDVDHPAAVSSPQR | SEQ ID NO: 59 |
| 61 | SAMSN1 | Q9N518 | Adaptor/scaffold | Y179 | VHTDFTPSPyDTDSLK | SEQ ID NO: 60 |
| 62 | Shb | Q15464 | Adaptor/scaffold | Y333 | VTIADDySDPFDAK | SEQ ID NO: 61 |
| 63 | SHEP1 | Q8N5H7 | Adaptor/scaffold | S440 | VHAAPAAPSATALPAsPVAR | SEQ ID NO: 62 |
| 64 | SHEP1 | Q8N5H7 | Adaptor/scaffold | Y487 | ASPSPSLSSySDPDSGHYCQLQPPVR | SEQ ID NO: 63 |
| 65 | SHEP1 | Q8N5H7 | Adaptor/scaffold | Y495 | ASPSPSLSSYSDPDSGHyCQLQPPVR | SEQ ID NO: 64 |
| 66 | SLAP-130 | O15117 | Adaptor/scaffold | Y571 | TTAVEIDyDSLK | SEQ ID NO: 65 |
| 67 | SLY | O75995 | Adaptor/scaffold | Y189 | VHTDFTPSPyDHDSLK | SEQ ID NO: 66 |
| 68 | Spinophilin | Q96SB3 | Adaptor/scaffold | Y23 | SAyEAGIQALKPPDAPGPDEAPK | SEQ ID NO: 67 |
| 69 | STS-1 | Q8TF42 | Adaptor/scaffold | Y20 | EELySKVTPRRNRQQRPGTIK | SEQ ID NO: 68 |
| 70 | TEM6 | Q8IZW7 | Adaptor/scaffold | S850 | ESMCSTPAFPVsPETPYVK | SEQ ID NO: 69 |
| 71 | tensin 1 | Q9HBL0 | Adaptor/scaffold | Y1404 | AGSLPNyATINGK | SEQ ID NO: 70 |
| 72 | TSAd | Q9NP31 | Adaptor/scaffold | Y280 | PKPSNPIyNEPDEPIAFYAMGR | SEQ ID NO: 71 |
| 73 | TSAd | Q9NP31 | Adaptor/scaffold | Y290 | PKPSNPIYNEPDEPIAFyAMGR | SEQ ID NO: 72 |
| 74 | ZO1 | Q07157 | Adaptor/scaffold | Y1423 | RYEPIQATPPPPPLPSQyAQPSQPVTSASLHIHSK | SEQ ID NO: 73 |
| 75 | ZO1 | Q07157 | Adaptor/scaffold | Y576 | AEQLASVQyTLPK | SEQ ID NO: 74 |
| 76 | Z02 | Q9UDY2 | Adaptor/scaffold | Y1118 | IEIAQKHPDIyAVPIK | SEQ ID NO: 75 |
| 77 | Z02 | Q9UDY2 | Adaptor/scaffold | Y423 | RQQySDQDYHSSTEK | SEQ ID NO: 76 |
| 78 | ZO2 | Q9UDY2 | Adaptor/scaffold | Y428 | RQQYSDQDyHSSTEK | SEQ ID NO: 77 |
| 79 | BAG3 | O95817 | Apoptosis | Y240 | THYPAQQGEyQTHQPVYHK | SEQ ID NO: 78 |
| 80 | BCL7C | O43770 | Apoptosis | S114 | GTEPsPGGTPQPSRPVSPAGPPEGVPEEAQPPR | SEQ ID NO: 79 |
| 81 | SET | Q01105 | Apoptosis | Y146 | DFYFDENPyFENK | SEQ ID NO: 80 |
| 82 | annexin A6 | P08133 | Calcium-binding | Y29 | KYRGSIHDFPGFDPNQDAEALy | SEQ ID NO: 81 |
| | | | protein | | | |
| 83 | REPS1 | Q96D71 | Calcium-binding | Y64 | HAASySSDSENQGSYSGVIPPPPGR | SEQ ID NO. 82 |
| | | | protein | | | |
| 84 | REPS1 | Q96D71 | Calcium-binding | Y74 | ASYSSDSENQGSySGVIPPPPGRGQVKKG | SEQ ID NO: 83 |
| | | | protein | | | |
| 85 | MDC1 | Q14676 | Cell cycle | S794 | AIPGDQHPEsPVHTEPMGIQGR | SEQ ID NO: 84 |
| | | | regulation | | | |
| 86 | IcIn | P54105 | Channel | Y214 | TEDSIRDyEDGMEVDTTPTVAGQFEDADVDH | SEQ ID NO: 85 |
| 87 | nAChR | P32297 | Channel | Y219 | yNCCEEIYPDITYSLYIR | SEQ ID NO: 86 |
| | alpha3 | | | | | |
| 88 | nAChR | P32297 | Channel | Y226 | YNCCEEIyPDITYSLYIR | SEQ ID NO: 87 |
| | alpha3 | | | | | |
| 89 | CCT-theta | P50990 | Chaperone | Y30 | HFSGLEEAVyR | SEQ ID NO: 88 |
| 90 | CCT-theta | P50990 | Chaperone | Y505 | GILDTYLGKyWAIK | SEQ ID NO: 89 |
| 91 | FKBP4 | Q02790 | Chaperone | Y219 | GEHSIVyLKPSYAFGSVGK | SEQ ID NO: 90 |
| 92 | HSP70 | P08107 | Chaperone | Y41 | TTPSyVAFTDTER | SEQ ID NO: 91 |
| 93 | HSP70 | P08107 | Chaperone | Y611 | ELEQVCNPIISGLyQGAGGPGPGGFGAQGPK | SEQ ID NO: 92 |
| 94 | HSP90-beta | P08238 | Chaperone | Y595 | LVSSPCCIVTSTyGWTANMER | SEQ ID NO: 93 |
| 95 | SGTA | O43765 | Chaperone | Y9 | MDNKKRLAyAIIQFLHDQLR | SEQ ID NO: 94 |
| 96 | TBCB | Q99426 | Chaperone | Y107 | VEKyTISQEAYDQR | SEQ ID NO: 95 |
| 97 | calponin | Q99349 | Contractile | Y302 | YCPQGTVADGAPSGTGDCPDPGEVPEYPPYyQEEAGY | SEQ ID NO: 96 |
| | 2 | | | | | |
| 98 | actin, | P02568 | Cytoskeletal | Y93 | IWHHTFyNELR | SEQ ID NO: 97 |
| | alpha 1 | | protein | | | |
| 99 | actin, | P02570 | Cytoskeletal | Y91 | WHHTFyNELRVAPEEHPV | SEQ ID NO: 98 |
| | beta | | protein | | | |
| 100 | actin, | P63261 | Cytoskeletal | Y294 | KDLyANTVLSGGTTMYPGLADR | SEQ ID NO: 99 |
| | gamma 1 | | protein | | | |
| 101 | ADAM18 | Q9R157 | Cytoskeletal | Y47 | VTyVITIDGKPYSLHLR | SEQ ID NO: 100 |
| | | | protein | | | |
| 102 | adducin, | P35612 | Cytoskeletal | Y489 | IENPNQFVPLyTDPQEVLEMR | SEQ ID NO: 101 |
| | beta | | protein | | | |
| 103 | Arp3 | P32391 | Cytoskeletal | Y202 | DITyFIQQLLR | SEQ ID NO: 102 |
| | | | protein | | | |
| 104 | CLASP2 | O75122 | Cytoskeletal | Y1052 | DYNPyNYSDSISPFNK | SEQ ID NO: 103 |
| | | | protein | | | |
| 105 | cofilin 1 | P23528 | Cytoskeletal | Y68 | NIILEEGKEILVGDVGQTVDDPyATFVK | SEQ ID NO. 104 |
| | | | protein | | | |
| 106 | cofilin 1 | P23528 | Cytoskeletal | Y85 | YALyDATYETKESK | SEQ ID NO: 105 |
| | | | protein | | | |
| 107 | cofilin 1 | P23528 | Cytoskeletal | Y89 | YALYDATyETKESK | SEQ ID NO: 106 |
| | | | protein | | | |
| 108 | cortactin | Q60598 | Cytoskeletal | Y334 | NASTFEEVVQVPSAyQK | SEQ ID NO: 107 |
| | | | protein | | | |
| 109 | DAL-1 | Q9Y2J2 | Cytoskeletal | Y203 | yYLCLQLRDDIVSGR | SEQ ID NO: 108 |
| | | | protein | | | |
| 110 | DAL-1 | Q9Y2J2 | Cytoskeletal | Y204 | YyLCLQLRDDIVSGR | SEQ ID NO; 109 |
| | | | protein | | | |
| 111 | Emerin | P50402 | Cytoskeletal | Y155 | LIyGQDSAYQSIAHYRPISNVSR | SEQ ID NO: 110 |
| | | | protein | | | |
| 112 | Emerin | P50402 | Cytoskeletal | Y161 | LIYGQDSAyQSIAHYRPISNVSR | SEQ ID NO: 111 |
| | | | protein | | | |
| 113 | Emerin | P50402 | Cytoskeletal | Y181 | SSLGLSyYPTSSTSSVSSSSSSPSSWLTR | SEQ ID NO: 112 |
| | | | protein | | | |
| 114 | Emerin | P50402 | Cytoskeletal | Y74 | GDADMyDLPKKEDALLYQSK | SEQ ID NO: 113 |
| | | | protein | | | |
| 115 | Emerin | P50402 | Cytoskeletal | Y94 | GYNDDyYEESYFTTR | SEQ ID NO: 114 |
| | | | protein | | | |
| 116 | eplin | Q9UHB6 | Cytoskeletal | S362 | SEVQQPVHPKPLsPDSR | SEQ ID NO: 115 |
| | | | protein | | | |
| 117 | eplin | Q9UHB6 | Cytoskeletal | S490 | ETPHsPGVEDAPIAK | SEQ ID NO: 116 |
| | | | protein | | | |
| 118 | Erbin | Q96RT1 | Cytoskeletal | Y1042 | ANTAyHLHQR | SEQ ID NO: 117 |
| | | | protein | | | |
| 119 | Erbin | Q96RT1 | Cytoskeletal | Y1164 | TMSVSDFNySR | SEQ ID NO: 118 |
| | | | protein | | | |
| 120 | ezrin | P15311 | Cytoskeletal | Y423 | SQEQLAAELAEyTAK | SEQ ID NO: 119 |
| | | | protein | | | |
| 121 | ezrin | P15311 | Cytoskeletal | Y477 | TAPPPPPPPVyEPVSY | SEQ ID NO: 120 |
| | | | protein | | | |
| 122 | Filamin A | P21333 | Cytoskeletal | Y1261 | LQVEPAVDTSGVQCyGPGIEGQGVFR | SEQ ID NO: 121 |
| | | | protein | | | |
| 123 | H4 | Q16204 | Cytoskeletal | S367 | TVSSPIPYTPSPSSSRPIsPGLSYASHTVGFTPPTSLTR | SEQ ID NO: 122 |
| | (D10S170) | | protein | | | |
| 124 | lamin B1 | P20700 | Cytoskeletal | S22 | AGGPTTPLsPTR | SEQ ID NO: 123 |
| | | | protein | | | |
| 125 | lamin B2 | Q03252 | Cytoskeletal | S17 | AGGPATPLsPTR | SEQ ID NO: 124 |
| | | | protein | | | |
| 126 | Leupaxin | O60711 | Cytoskeletal | Y62 | VQLVyATNIQEPNVYSEVQEPK | SEQ ID NO: 125 |
| | | | protein | | | |
| 127 | Leupaxin | O60711 | Cytoskeletal | Y72 | VQLVYATNIQEPNVySEVQEPK | SEQ ID NO: 126 |
| | | | protein | | | |
| 128 | L-plastin | P13796 | Cytoskeletal | Y276 | WANyHLENAGCNK | SEQ ID NO: 127 |
| | | | protein | | | |
| 129 | L-plastin | P13796 | Cytoskeletal | Y28 | VDTDGNGyISFNELNDLFK | SEQ ID NO: 128 |
| | | | protein | | | |
| 130 | L-plastin | P13796 | Cytoskeletal | Y598 | VyALPEDLVEVNPK | SEQ ID NO: 129 |
| | | | protein | | | |
| 131 | LPP | Q93052 | Cytoskeletal | Y234 | SAQPSPHyMAGPSSGQIYGPGPR | SEQ ID NO: 130 |
| | | | protein | | | |
| 132 | moesin | P26038 | Cytoskeletal | Y115 | EGILNDDIyCPPETAVLLASYAVQSK | SEQ ID NO: 131 |
| | | | protein | | | |
| 133 | Plakophilin | Q9Y446 | Cytoskeletal | Y84 | GQyHTLQAGFSSR | SEQ ID NO: 132 |
| | 3 | | protein | | | |
| 134 | Plakophilin | Q99569 | Cytoskeletal | Y487 | NNYALNTTATYAEPYRPIQyR | SEQ ID NO: 133 |
| | 4 | | protein | | | |
| 135 | plectin 1 | Q15149 | Cytoskeletal | S4396 | SSSVGSSSSYPIsPAVSR | SEQ ID NO: 134 |
| | | | protein | | | |
| 136 | plectin 1 | Q15149 | Cytoskeletal | Y4612 | LLEAAAQSTKGYySPYSVSGSGSTAGSR | SEQ ID NO: 135 |
| | | | protein | | | |
| 137 | similar | XP_377631 | Cytoskeletal | Y224 | EIMPHIREKLCyITLDFEKEMATAASSSSLEK | SEQ ID NO: 136 |
| | to beta | | protein | | | |
| | actin | | | | | |
| 138 | Spectrin- | Q13813 | Cytoskeletal | Y1411 | AGTFQAFEQFGQQLLAHGHyASPEIK | SEQ ID NO: 137 |
| | alphall | | protein | | | |
| 139 | Spectrin- | Q13813 | Cytoskeletal | Y2423 | ALSSEGKPyVTKEELYQNLTR | SEQ ID NO: 138 |
| | alphall | | protein | | | |
| 140 | Spectrin- | Q01082 | Cytoskeletal | Y1730 | EVVAGSHELGQDyEHVTMLQER | SEQ ID NO: 139 |
| | betall | | protein | | | |
| 141 | Spectrin- | Q01082 | Cytoskeletal | Y199 | IVSSSDVGHDEySTQSLVK | SEQ ID NO: 140 |
| | betall | | protein | | | |
| 142 | talin 1 | Q9Y490 | Cytoskeletal | Y199 | FFySDQNVDSR | SEQ ID NO: 141 |
| | | | protein | | | |
| 143 | talin 1 | Q9Y490 | Cytoskeletal | Y436 | KSTVLQQQyNR | SEQ ID NO: 142 |
| | | | protein | | | |
| 144 | tubulin, | P05209 | Cytoskeletal | Y210 | FMVDNEAIyDICRRNLDIERPT | SEQ ID NO: 143 |
| | alpha-1 | | protein | | | |
| 145 | tubulin, | P05209 | Cytoskeletal | Y224 | NLDIERPTyTNLNR | SEQ ID NO: 144 |
| | alpha-1 | | protein | | | |
| 146 | tubulin, | P05209 | Cytoskeletal | Y432 | SEAREDMMLEKDyEEVGVDSVEGEGEEEGEEY | SEQ ID NO: 145 |
| | alpha-1 | | protein | | | |
| 147 | tubulin, | P07437 | Cytoskeletal | Y340 | NSSyFVEWIPNNVK | SEQ ID NO: 146 |
| | beta-1 | | protein | | | |
| 148 | vimentin | P08670 | Cytoskeletal | Y29 | SyVTTSTR | SEQ ID NO: 147 |
| | | | protein | | | |
| 149 | vinculin | P18206 | Cytoskeletal | Y821 | SFLDSGyR | SEQ ID NO: 148 |
| | | | protein | | | |
| 150 | zyxin | Q15942 | Cytoskeletal | Y172 | VSSGyVPPPVATPFSSK | SEQ ID NO: 149 |
| | | | protein | | | |
| 151 | utrophin | P46939 | Dystrophin | Y2599 | QMPIGGDVPALQLQyDHCK | SEQ ID NO: 150 |
| | | | complex | | | |
| 152 | aldolase A | P04075 | Enzyme, cellular | Y328 | AWGGKEENLKAAQEEyIKR | SEQ ID NO: 151 |
| | | | metabolism | | | |
| 153 | AMPD2 | Q01433 | Enzyme, cellular | Y197 | TDSDSDLQLyKEQGEGQGDR | SEQ ID NO: 152 |
| | | | metabolism | | | |
| 154 | CRMP-1 | Q14194 | Enzyme, cellular | Y504 | GMYDGPVyEVPATPK | SEQ ID NO: 153 |
| | | | metabolism | | | |
| 155 | CTP | P17812 | Enzyme, cellular | Y53 | KIDPYINIDAGTFSPyEHGEV | SEQ ID NO: 154 |
| | synthetase | | metabolism | | | |
| 156 | DOT1L | Q8TEK3 | Enzyme, cellular | S1001 | NSLPAsPAHQLSSSPR | SEQ ID NO: 155 |
| | | | metabolism | | | |
| 157 | G6PD | P11413 | Enzyme, cellular | Y423 | KPGMFFNPEESELDLTyGNRYK | SEQ ID NO: 156 |
| | | | metabolism | | | |
| 158 | GDE | P35573 | Enzyme, cellular | Y584 | EAMSAyNSHEEGR | SEQ ID NO: 157 |
| | | | metabolism | | | |
| 159 | glycogenin | P46976 | Enzyme, cellular | Y331 | WEQGQADyMGADSFDNIKR | SEQ ID NO: 158 |
| | | | metabolism | | | |
| 160 | GOT1 | P17174 | Enzyme, cellular | Y70 | IANDNSLNHEyLPILGLAEFR | SEQ ID NO: 159 |
| | | | metabolism | | | |
| 161 | LDH-A | P00338 | Enzyme, cellular | Y144 | LLIVSNPVDILTyVAWK | SEQ ID NO: 160 |
| | | | metabolism | | | |
| 162 | LDH-A | P00338 | Enzyme, cellular | Y9 | DQLIyNLLKEEQTPQNK | SEQ ID NO: 161 |
| | | | metabolism | | | |
| 163 | MRGBP | Q9NV56 | Enzyme, cellular | S195 | VLTANSNPSsPSAAK | SEQ ID NO: 162 |
| | | | metabolism | | | |
| 164 | NEDD4L | Q7Z5N3 | Enzyme, cellular | S479 | DTLSNPQsPQPSPYNSPKPQHK | SEQ ID NO: 163 |
| | | | metabolism | | | |
| 165 | NEDD4L | Q7Z5N3 | Enzyme, cellular | S483 | DTLSNPQSPQPsPYNSPKPQHK | SEQ ID NO: 164 |
| | | | metabolism | | | |
| 166 | NEDD4L | Q7Z5N3 | Enzyme, cellular | S487 | DTLSNPQSPQPSPYNsPKPQHK | SEQ ID NO: 165 |
| | | | metabolism | | | |
| 167 | PDHA1 | P08559 | Enzyme, cellular | Y289 | yHGHSMSDPGVSYR | SEQ ID NO: 166 |
| | | | metabolism | | | |
| 168 | PDHA1 | P08559 | Enzyme, cellular | Y301 | YHGHSMSDPGVSyR | SEQ ID NO: 167 |
| | | | metabolism | | | |
| 169 | PGM1 | P36871 | Enzyme, cellular | Y352 | IALyETPTGWK | SEQ ID NO: 168 |
| | | | metabolism | | | |
| 170 | phospho | P18669 | Enzyme, cellular | Y91 | HyGGLTGLNK | SEQ ID NO: 169 |
| | glycerate | | metabolism | | | |
| | mutase 1 | | | | | |
| 171 | PRMT1 | Q99873 | Enzyme, cellular | Y299 | TGFSTSPESPyTHWK | SEQ ID NO: 170 |
| | | | metabolism | | | |
| 172 | PTDSS1 | P48651 | Enzyme, cellular | Y416 | EKTySECEDGTYSPEISWHHR | SEQ ID NO: 171 |
| | | | metabolism | | | |
| 173 | PTDSS1 | P48651 | Enzyme, cellular | Y424 | TYSECEDGTySPEISWHHR | SEQ ID NO: 172 |
| | | | metabolism | | | |
| 174 | pyruvate | P14786 | Enzyme, cellular | Y147 | ITLDNAyMEKCDENILWLDYK | SEQ ID NO: 173 |
| | kinase M | | metabolism | | | |
| 175 | pyruvate | P14786 | Enzyme, cellular | Y369 | AEGSDVANAVLDGADCIMLSGETAKGDyPLEAVR | SEQ ID NO: 174 |
| | kinase M | | metabolism | | | |
| 176 | SAHH | P23526 | Enzyme, cellular | Y193 | SKFDNLyGCR | SEQ ID NO: 175 |
| | | | metabolism | | | |
| 177 | thiamine | Q9BU02 | Enzyme, cellular | Y30 | LQELGGTLEyR | SEQ ID NO: 176 |
| | triphos- | | metabolism | | | |
| | phatase | | | | | |
| 178 | GCET2 | Q8N6F7 | expressed in | Y107 | VLCTRPSGNSAEEYyENVPCK | SEQ ID NO: 177 |
| | | | germinal center | | | |
| 179 | Mx1 | P20591 | G protein | Y128 | GKVSYQDyEIEISDASEVEKEINK | SEQ ID NO: 178 |
| 180 | Rab GDI | P31150 | G protein | Y38 | LHMDRNPyYGGES | SEQ ID NO: 179 |
| | alpha | | regulator | | | |
| 181 | Rab GDI | P50395 | G protein | Y203 | LYRTDDYLDQPCyETINR | SEQ ID NO: 180 |
| | beta | | regulator | | | |
| 182 | ARF | Q9NP61 | GTPase | Y378 | SSSESSWDDGADSyWK | SEQ ID NO: 181 |
| | GAP 3 | | activating | | | |
| | | | protein | | | |
| 183 | centaurin- | Q8WZ64 | GTPase | Y473 | HSYPLSSTSGNADSSAVSSQAISPyACFYGASAK | SEQ ID NO: 182 |
| | delta 1 | | activating | | | |
| | | | protein | | | |
| 184 | centaurin- | Q8WZ64 | GTPase | Y77 | MQDIPIyANVHK | SEQ ID NO: 183 |
| | delta 1 | | activating | | | |
| | | | protein | | | |
| 185 | centaurin- | Q96P48 | GTPase | Y423 | HySVVLPTVSHSGFLYK | SEQ ID NO: 184 |
| | delta 2 | | activating | | | |
| | | | protein | | | |
| 186 | centaurin- | Q96P48 | GTPase | Y437 | HYSVVLPTVSHSGFLyK | SEQ ID NO: 185 |
| | delta 2 | | activating | | | |
| | | | protein | | | |
| 187 | centaurin- | Q96P48 | GTPase | Y661 | AAASMGDTLSEQQLGDSDIPVIVyR | SEQ ID NO: 186 |
| | delta 2 | | activating | | | |
| | | | protein | | | |
| 188 | GIT2 | Q14161 | GTPase | Y592 | QNSTPESDyDNTACDPEPDDTGSTR | SEQ ID NO: 187 |
| | | | activating | | | |
| | | | protein | | | |
| 189 | IQGAP1 | P46940 | GTPase | Y654 | SPDVGLyGVIPECGETYHSDLAEAK | SEQ ID NO: 188 |
| | | | activating | | | |
| | | | protein | | | |
| 190 | RGS14 | O43566 | GTPase | S478 | ATHPPPAsPSSLVK | SEQ ID NO: 189 |
| | | | activating | | | |
| | | | protein | | | |
| 191 | similar to | XP_113914 | GTPase | Y28 | ALPAQVDDPPEPVyANIER | SEQ ID NO: 190 |
| | RGS12 | | activating | | | |
| | | | protein | | | |
| 192 | SIPA1L1 | O43166 | GTPase | S162 | FLMPEAYPSsPR | SEQ ID NO: 191 |
| | | | activating | | | |
| | | | protein | | | |
| 193 | GEF-H1 | Q8TDA3 | Guanine | Y125 | ERPSSAIyPSDSFR | SEQ ID NO: 192 |
| | | | nucleotide | | | |
| | | | exchange | | | |
| | | | factor | | | |
| 194 | PSD4 | O95621 | Guanine | S134 | QNTASPGsPVNSHLPGSPK | SEQ ID NO: 193 |
| | | | nucleotide | | | |
| | | | exchange | | | |
| | | | factor | | | |
| 195 | PSD4 | O95621 | Guanine | S138 | QNTASPGSPVNsHLPGSPK | SEQ ID NO: 194 |
| | | | nucleotide | | | |
| | | | exchange | | | |
| | | | factor | | | |
| 196 | RCC1- | Q96151 | Guanine | Y216 | EGVFSMGNNSHGQCGRKVVEDEVySESHK | SEQ ID NO: 195 |
| | like GEF | | nucleotide | | | |
| | | | exchange | | | |
| | | | factor | | | |
| 197 | TD-60 | Q9P258 | Guanine | Y325 | GNLYSFGCPEyGQLGHNSDGK | SEQ ID NO: 196 |
| | | | nucleotide | | | |
| | | | exchange | | | |
| | | | factor | | | |
| 198 | VAV1 | P15498 | Guanine | Y844 | VGWFPANYVEEDYSEyC | SEQ ID NO: 197 |
| | | | nucleotide | | | |
| | | | exchange | | | |
| | | | factor | | | |
| 199 | CD19 | P15391 | Immunoglobulin | Y348 | VTPPPGSGPQNQyGNVLSLPTPTSGLGR | SEQ ID NO: 198 |
| | | | superfamily | | | |
| 200 | CD22 | P20273 | Immunoglobulin | Y822 | KRQVGDYENVIPDFPEDEGIHySELIQF | SEQ ID NO: 199 |
| | | | superfamily | | | |
| 201 | CD84 | O15430 | Immunoglobulin | Y279 | NAQPTESRIyDEIPQSK | SEQ ID NO: 200 |
| | | | superfamily | | | |
| 202 | Fc-epsilon | P30273 | Immunoglobulin | Y65 | SDGVyTGLSTR | SEQ ID NO: 201 |
| | RI-gamma | | superfamily | | | |
| 203 | SLAMF7 | Q9NY08 | Immunoglobulin | Y304 | TILKEDPANTVySTVEIPK | SEQ ID NO: 202 |
| | | | superfamily | | | |
| 204 | IkB- | O00221 | Inhibitor | Y16 | KGPDEAEESQyDSGIESLR | SEQ ID NO: 203 |
| | epsilon | | protein | | | |
| 205 | ITIH1 | P19827 | Inhibitor | Y327 | ILGDMQPGDyFDLVLFGTR | SEQ ID NO: 204 |
| | | | protein | | | |
| 206 | LANP-L | Q9BTT0 | Inhibitor | Y235 | EEIQDEEDDDDyVEEGEEEEEEEEGGLRGEK | SEQ ID NO: 205 |
| | | | protein | | | |
| 207 | TRAIP | O75766 | Inhibitor | Y573 | ELTyQNTDLSEIKEEEQVK | SEQ ID NO: 206 |
| | | | protein | | | |
| 208 | PI3K | P42338 | Kinase, lipid | Y503 | KQPyYYPPFDK | SEQ ID NO: 207 |
| | p110-beta | | | | | |
| 209 | PI3K | P42338 | Kinase, lipid | Y504 | KQPYyYPPFDK | SEQ ID NO: 208 |
| | p110-beta | | | | | |
| 210 | PI3K | P42338 | Kinase, lipid | Y505 | KQPYYyPPFDK | SEQ ID NO: 209 |
| | p110-beta | | | | | |
| 211 | PI3K | P42338 | Kinase, lipid | Y772 | EALSDLQSPLNPCVILSELyVEK | SEQ ID NO: 210 |
| | p110-beta | | | | | |
| 212 | PI3K | O00329 | Kinase, lipid | Y484 | SNPNTDSAAALLICLPEVAPHPVyYPALEK | SEQ ID NO: 211 |
| | P110-delta | | | | | |
| 213 | PI3K | O00329 | Kinase, lipid | Y485 | SNPNTDSAAALLICLPEVAPHPVYyPALEK | SEQ ID NO: 212 |
| | P110-delta | | | | | |
| 214 | PI3K | O00329 | Kinase, lipid | Y524 | GSGELyEHEKDLVWK | SEQ ID NO: 213 |
| | P110-delta | | | | | |
| 215 | PI3K | O00329 | Kinase, lipid | Y936 | ERVPFILTyDFVHVIQQGK | SEQ ID NO: 214 |
| | P110-delta | | | | | |
| 216 | PI3K p85- | P27986 | Kinase, lipid | Y452 | LHEyNTQFQEK | SEQ ID NO: 215 |
| | alpha | | | | | |
| 217 | PI3K p85- | P27986 | Kinase, lipid | Y467 | SREYDRLyEEYTR | SEQ ID NO: 216 |
| | alpha | | | | | |
| 218 | PI3K p85- | O00459 | Kinase, lipid | Y453 | VYHQQyQDK | SEQ ID NO: 217 |
| | beta | | | | | |
| 219 | PIP5K | Q9Y2I7 | Kinase, lipid | Y1773 | GADSAYyQVGQTGK | SEQ ID NO: 218 |
| 220 | OSBPL11 | Q9BXB4 | Lipid binding | Y62 | GWQYSDHMENVyGYLMK | SEQ ID NO: 219 |
| | | | protein | | | |
| 221 | SSBP1 | Q04837 | Mitochondrial | Y73 | SGDSEVyQLGDVSQK | SEQ ID NO: 220 |
| 222 | DRP1 | O00429 | Motor protein | S616 | SKPIPIMPAsPQKGHAVNLLDVPVPVAR | SEQ ID NO: 221 |
| 223 | MYH9 | P35579 | Motor protein | Y151 | KRHEMPPHIyAITDTAYR | SEQ ID NO: 222 |
| 224 | MYH9 | P35579 | Motor protein | Y754 | ALELDSNLyRIGQSK | SEQ ID NO: 223 |
| 225 | MYL6 | P60660 | Motor protein | Y85 | NKDQGTyEDYVEGLR | SEQ ID NO: 224 |
| 226 | MYL6 | P60660 | Motor protein | Y88 | NKDQGTYEDyVEGLR | SEQ ID NO: 225 |
| 227 | Sec24C | P53992 | Motor protein | Y296 | GPQPNyESPYPGAPTFGSQPGPPQPLPPK | SEQ ID NO: 226 |
| 228 | Sec24C | P53992 | Motor protein | Y300 | GPQPNYESPyPGAPTFGSQPGPPQPLPPK | SEQ ID NO: 227 |
| 229 | DDX5 | P17844 | Nuclear | Y202 | STCIyGGAPK | SEQ ID NO. 228 |
| 230 | Dicer1 | Q9UPY3 | Nuclear | Y1428 | APKEEADyEDDFLEYDQEHIR | SEQ ID NO: 229 |
| 231 | Dicer1 | Q9UPY3 | Nuclear | Y1435 | APKEEADYEDDFLEyDQEHIR | SEQ ID NO: 230 |
| 232 | HELZ | P42694 | Nuclear | Y1353 | HINLPLPAPHAQyAIPNR | SEQ ID NO: 231 |
| 233 | senataxin | Q7Z333 | Nuclear | S1663 | NSCNVLHPQsPNNSNR | SEQ ID NO: 232 |
| 234 | Bright | Q99856 | Nuclear, DNA | S77 | AAAAGLGHPAsPGGSEDGPPGSEEEDAAR | SEQ ID NO: 233 |
| | | | repair | | | |
| 235 | KAB1 | Q9UQ09 | Nuclear, DNA | Y240 | QVEEQSAAANEEVLFPFCREPSyFEIPTK | SEQ ID NO: 234 |
| | | | repair | | | |
| 236 | Nedd4-BP2 | Q86UW6 | Nuclear, DNA | Y1244 | NNNDILPNSQEELLySSK | SEQ ID NO: 235 |
| | | | repair | | | |
| 237 | ARPP-19 | P56211 | Nuclear, RNA | Y58 | LQKGQKyFDSGDYNMAK | SEQ ID NO: 236 |
| | | | binding | | | |
| 238 | CIRBP | Q14011 | Nuclear, RNA | Y141 | SGGYGGSRDyYSSR | SEQ ID NO: 237 |
| | | | binding | | | |
| 239 | CIRBP | Q14011 | Nuclear, RNA | Y142 | SGGYGGSRDYySSR | SEQ ID NO: 238 |
| | | | binding | | | |
| 240 | CIRBP | Q14011 | Nuclear, RNA | Y160 | SSGGSyRDSYDSYATHNE | SEQ ID NO: 239 |
| | | | binding | | | |
| 241 | CIRBP | Q14011 | Nuclear, RNA | Y164 | SSGGSYRDSyDSYATHNE | SEQ ID NO: 240 |
| | | | binding | | | |
| 242 | CIRBP | Q14011 | Nuclear, RNA | Y167 | SSGGSYRDSYDSyATHNE | SEQ ID NO: 241 |
| | | | binding | | | |
| 243 | FIP1L1 | Q9H077 | Nuclear, RNA | Y95 | TGAPQyGSYGTAPVNLNIK | SEQ ID NO: 242 |
| | | | binding | | | |
| 244 | FIP1L1 | Q9H077 | Nuclear, RNA | Y98 | TGAPQYGSyGTAPVNLNIK | SEQ ID NO: 243 |
| | | | binding | | | |
| 245 | hnRNP | P22626 | Nuclear, RNA | S259 | GFGDGYNGYGGGPGGGNFGGsPGYGGGR | SEQ ID NO: 244 |
| | A2/B1 | | binding | | | |
| 246 | hnRNP | P22626 | Nuclear, RNA | Y347 | NMGGPYGGGNYGPGGSGGSGGyGGR | SEQ ID NO: 245 |
| | A2/B1 | | binding | | | |
| 247 | hnRNP | P51991 | Nuclear, RNA | Y373 | SSGSPYGGGYGSGGGSGGyGSR | SEQ ID NO: 246 |
| | A3 | | binding | | | |
| 248 | hnRNP H | P31943 | Nuclear, RNA | S104 | HTGPNsPDTANDGFVR | SEQ ID NO: 247 |
| | | | binding | | | |
| 249 | hnRNP H | P31943 | Nuclear, RNA | Y266 | DLNyCFSGMSDHR | SEQ ID NO: 248 |
| | | | binding | | | |
| 250 | hnRNP R | O43390 | Nuclear, RNA | Y435 | STAYEDYyYHPPPR | SEQ ID NO: 249 |
| | | | binding | | | |
| 251 | hnRNP R | O43390 | Nuclear, RNA | Y436 | STAYEDYYyHPPPR | SEQ ID NO: 250 |
| | | | binding | | | |
| 252 | hnRNP-A1 | P09651 | Nuclear, RNA | Y365 | NQGGYGGSSSSSSyGSGR | SEQ ID NO: 251 |
| | | | binding | | | |
| 253 | hnRNP-I | P26599 | Nuclear, RNA | Y127 | GQPIyIQFSNHK | SEQ ID NO: 252 |
| | | | binding | | | |
| 254 | MpI | Q96NF9 | Nuclear, RNA | Y326 | HNPTVTGQQEQTyLPK | SEQ ID NO: 253 |
| | binding | | binding | | | |
| | protein | | | | | |
| 255 | PABP 1 | P11940 | Nuclear, RNA | Y116 | ALyDTFSAFGNILSCK | SEQ ID NO: 254 |
| | | | binding | | | |
| 256 | PAI- | Q8NC51 | Nuclear, RNA | Y207 | SSFSHySGLK | SEQ ID NO: 255 |
| | RBP1 | | binding | | | |
| 257 | PCBP2 | Q15366 | Nuclear, RNA | Y236 | TIQGQyAIPQPDLTKL | SEQ ID NO: 256 |
| | | | binding | | | |
| 258 | RBM3 | P98179 | Nuclear, RNA | Y125 | YYDSRPGGyGYGYGR | SEQ ID NO: 257 |
| | | | binding | | | |
| 259 | SF2 | Q07955 | Nuclear, RNA | S198 | VKVDGPRsPSYGRSR | SEQ ID NO: 258 |
| | | | binding | | | |
| 260 | SF2 | Q07955 | Nuclear, RNA | S204 | VKVDGPRSPSYGRsR | SEQ ID NO: 259 |
| | | | binding | | | |
| 261 | SFRS9 | Q13242 | Nuclear, RNA | Y179 | SHEGETSyIR | SEQ ID NO: 260 |
| | | | binding | | | |
| 262 | snRNP 70 | P08621 | Nuclear, RNA | Y126 | EFEVyGPIKR | SEQ ID NO: 261 |
| | | | binding | | | |
| 263 | SRm160 | Q8IY83 | Nuclear, RNA | S773 | KPPAPPSPVQsQSPSTNWSPAVPVKK | SEQ ID NO: 262 |
| | | | binding | | | |
| 264 | SRm300 | Q9UQ35 | Nuclear, RNA | S323 | GEGDAPFSEPGTTSTQRPSsPETATK | SEQ ID NO: 263 |
| | | | binding | | | |
| 265 | SRp46 | Q9BRL6 | Nuclear, RNA | S26 | VDNLTYRTsPDSLRR | SEQ ID NO: 264 |
| | | | binding | | | |
| 266 | 53BP1 | Q12888 | Nuclear, | S1094 | QSQQPMKPIsPVKDPVSPASQK | SEQ ID NO: 265 |
| | | | transcription | | | |
| 267 | 53BP1 | Q12888 | Nuclear, | S1101 | QSQQPMKPISPVKDPVsPASQK | SEQ ID NO: 266 |
| | | | transcription | | | |
| 268 | 53BP1 | Q12888 | Nuclear, | Y1523 | LLFDDGyECDVLGK | SEQ ID NO: 267 |
| | | | transcription | | | |
| 269 | 53BP2 | Q13625 | Nuclear, | Y350 | VAAVGPyIQSSTMPR | SEQ ID NO: 268 |
| | | | transcription | | | |
| 270 | CDA02 | Q9BY44 | Nuclear, | Y275 | TGASYyGEQTLHYIATNGESAVVQLPK | SEQ ID NO: 269 |
| | | | transcription | | | |
| 271 | CDA02 | Q9BY44 | Nuclear, | Y386 | LISKPVASDSTyFAWCPDGEHILTATCAPR | SEQ ID NO: 270 |
| | | | transcription | | | |
| 272 | Elf-1 | P32519 | Nuclear, | S187 | KTKPPRPDsPATTPNISVK | SEQ ID NO: 271 |
| | | | transcription | | | |
| 273 | ELG | Q9NXZ4 | Nuclear, | S220 | RPHsPEKAFSSNPVVR | SEQ ID NO: 272 |
| | | | transcription | | | |
| 274 | ERF | P50548 | Nuclear, | Y42 | KEEyQGVIAWQGDYGEFVIK | SEQ ID NO: 273 |
| | | | transcription | | | |
| 275 | ERF | P50548 | Nuclear, | Y52 | KEEYQGVIAWQGDyGEFVIK | SEQ ID NO: 274 |
| | | | transcription | | | |
| 276 | FBI1 | O95365 | Nuclear, | S511 | VRGGAPDPsPGATATPGAPAQPSSPDAR | SEQ ID NO: 275 |
| | | | transcription | | | |
| 277 | FOXN3 | O00409 | Nuclear, | S85 | SVsPVQDLDDDTPPSPAHSDMPYDAR | SEQ ID NO: 276 |
| | | | transcription | | | |
| 278 | FOXN3 | O00409 | Nuclear, | S97 | SVSPVQDLDDDTPPsPAHSDMPYDAR | SEQ ID NO: 277 |
| | | | transcription | | | |
| 279 | GRF-1 | Q9NRY4 | Nuclear, | Y1087 | SVSSSPWLPQDGFDPSDyAEPMDAVVKPR | SEQ ID NO: 278 |
| | | | transcription | | | |
| 280 | HAND2 | P61296 | Nuclear, | Y147 | LATSyIAYLMDLLAKDDQNGEAEAFK | SEQ ID NO: 279 |
| | | | transcription | | | |
| 281 | HAND2 | P61296 | Nuclear, | Y150 | LATSYIAyLMDLLAKDDQNGEAEAFK | SEQ ID NO: 280 |
| | | | transcription | | | |
| 282 | MLL | Q03164 | Nuclear, | S3515 | ALSSAVQASPTSPGGsPSSPSSGQR | SEQ ID NO: 281 |
| | | | transcription | | | |
| 283 | MLL2 | O14686 | Nuclear, | Y1669 | PFLQGGLPLGNLPSSSPMDSyPGLCQSPFLDSRER | SEQ ID NO: 282 |
| | | | transcription | | | |
| 284 | MTA2 | O94776 | Nuclear, | Y22 | VGDYVYFENSSSNPyLVR | SEQ ID NO: 283 |
| | | | transcription | | | |
| 285 | NFAT2 | O95644 | Nuclear, | Y709 | TYLPANVPIIKTEPTDDyEPAPTCGPVSQGL | SEQ ID NO: 284 |
| | | | transcription | | | |
| 286 | NIF3L1 | Q9GZT8 | Nuclear, | Y103 | VGIYSPHTAyDAAPQGVNNWLAK | SEQ ID NO: 285 |
| | | | transcription | | | |
| 287 | p66 beta | Q8WXI9 | Nuclear, | Y317 | TTSSAIyMNLASHIQPGTVNR | SEQ ID NO: 286 |
| | | | transcription | | | |
| 288 | PHF16 | Q92613 | Nuclear, | S566 | NSSTETDQQPHsPDSSSSVHSIR | SEQ ID NO: 287 |
| | | | transcription | | | |
| 289 | PTTG1IP | P53801 | Nuclear, | Y174 | KYGLFKEENPyAR | SEQ ID NO: 288 |
| | | | transcription | | | |
| 290 | RERE | Q9P2R6 | Nuclear, | S594 | KKQPAsPDGRTSPINEDIR | SEQ ID NO: 289 |
| | | | transcription | | | |
| 291 | RERE | Q9P2R6 | Nuclear, | S600 | KKQPASPDGRTsPINEDIR | SEQ ID NO: 290 |
| | | | transcription | | | |
| 292 | RNA pol | P24928 | Nuclear, | S1815 | YTPQsPTYTPSSPSYSPSSPSYSPTSPK | SEQ ID NO: 291 |
| | II largest | | transcription | | | |
| | subunit | | | | | |
| 293 | RNA pol | P24928 | Nuclear, | S1822 | YTPQSPTYTPSsPSYSPSSPSYSPTSPK | SEQ ID NO: 292 |
| | II largest | | transcription | | | |
| | subunit | | | | | |
| 294 | RNa pol | P24928 | Nuclear, | S1845 | YTPTSPsYSPSSPEYTPTSPK | SEQ ID NO: 293 |
| | II largest | | transcription | | | |
| | subunit | | | | | |
| 295 | RNA pol | P24928 | Nuclear, | S1850 | YTPTSPSYSPSsPEYTPTSPK | SEQ ID NO: 294 |
| | II largest | | transcription | | | |
| | subunit | | | | | |
| 296 | RPA40 | O15160 | Nuclear, | Y36 | NVHTTDFPGNYSGyDDAWDQDRFEK | SEQ ID NO: 295 |
| | | | transcription | | | |
| 297 | SHARP | Q96T58 | Nuclear, | S749 | RPQSPGASPSQAERLPsDSER | SEQ ID NO: 296 |
| | | | transcription | | | |
| 298 | similar to | XP_116612 | Nuclear, | Y396 | IIHTGEKPYKSKIMYTEENyKYEMKNVAK | SEQ ID NO: 297 |
| | KRAP ZFP | | transcription | | | |
| 299 | similar to | XP_116612 | Nuclear, | Y398 | IIHTGEKPYKSKIMYTEENYKyEMKNVAK | SEQ ID NO: 298 |
| | KRAB ZFP | | transcription | | | |
| 300 | SSBP2 | P81877 | Nuclear, | Y192 | QQGHPNMGGPMQRMTPPRGMVPLGPQNyGGAMR | SEQ ID NO: 299 |
| | | | transcription | | | |
| 301 | TAFII31 | Q16594 | Nuclear, | Y261 | KREDDDDDDDDDDDyDNL | SEQ ID NO: 300 |
| | | | transcription | | | |
| 302 | Tel | P41212 | Nuclear, | Y17 | ISyTPPESPVPSYASSTPLHVPVPR | SEQ ID NO: 301 |
| | | | transcription | | | |
| 303 | Tel | P41212 | Nuclear, | Y27 | ISYTPPESPVPSyASSTPLHVPVPR | SEQ ID NO: 302 |
| | | | transcription | | | |
| 304 | Tel | P41212 | Nuclear, | Y314 | NLSHREDLAy | SEQ ID NO: 303 |
| | | | transcription | | | |
| 305 | Tel | P41212 | Nuclear, | Y447 | TDRLEHLESQELDEQIyQEDEC | SEQ ID NO: 304 |
| | | | transcription | | | |
| 306 | Trap170 | O60244 | Nuclear, | S1112 | AGNWPGsPQVSGPSPAAR | SEQ ID NO: 305 |
| | | | transcription | | | |
| 307 | Trap170 | O60244 | Nuclear, | S1119 | AGNWPGSPQVSGPsPAAR | SEQ ID NO: 306 |
| | | | transcription | | | |
| 308 | TRIP6 | Q15654 | Nuclear, | Y131 | QAYEPPPPPAyR | SEQ ID NO: 307 |
| | | | transcription | | | |
| 309 | UKp68 | Q6PJT7 | Nuclear, | S620 | NGDECAYHHPIsPCKAFPNCK | SEQ ID NO: 308 |
| | | | transcription | | | |
| 310 | ZAP | Q7Z2W4 | Nuclear, | Y410 | KGTGLLSSDyR | SEQ ID NO: 309 |
| | | | transcription | | | |
| 311 | ZBED4 | O75132 | Nuclear, | S624 | TEVSETARPSsPDTR | SEQ ID NO: 310 |
| | | | transcription | | | |
| 312 | ZNF202 | O95125 | Nuclear, | Y425 | PyKCMECGKSYTR | SEQ ID NO: 311 |
| | | | transcription | | | |
| 313 | ZNF202 | O95125 | Nuclear, | Y434 | PYKCMECGKSyTR | SEQ ID NO: 312 |
| | | | transcription | | | |
| 314 | ZNF330 | Q9Y3S2 | Nuclear, | Y250 | QTGGEEGDGASGyDAYWK | SEQ ID NO: 313 |
| | | | transcription | | | |
| 315 | ZNF330 | Q9Y3S2 | Nuclear, | Y253 | QTGGEEGDGASGYDAyWK | SEQ ID NO: 314 |
| | | | transcription | | | |
| 316 | ZNF395 | Q9NPB2 | Nuclear, | Y280 | RKNSVKVMyKCLWPNCGKVLRSIVGIKR | SEQ ID NO: 315 |
| | | | transcription | | | |
| 317 | SHIP | Q92835 | Phosphatase, | Y864 | EKLyDFVKTER | SEQ ID NO: 316 |
| | | | lipid | | | |
| 318 | SHIP-2 | O15357 | Phosphatase, | Y987 | NSFNNPAYyVLEGVPHQLLPPEPPSPAR | SEQ ID NO: 317 |
| | | | lipid | | | |
| 319 | 2′-PDE | Q6L8Q7 | Phospho- | S220 | EAKPGAAEPEVGVPSSLSPSsPSSSWTETDVEER | SEQ ID NO: 318 |
| | | | diesterase | | | |
| 320 | cathepsin K | P43235 | Protease | Y307 | GSKHWIKNSWGESWGNKGyALLAR | SEQ ID NO: 319 |
| 321 | IRAP | Q9UIQ6 | Protease | Y70 | GLGEHEMEEDEEDyESSAK | SEQ ID NO: 320 |
| 322 | PSMA2 | P25787 | Protease | Y100 | KLAQQYYLVyQEPIPTAQLVQR | SEQ ID NO: 321 |
| 323 | PSMA2 | P25787 | Protease | Y75 | HIGLVySGMGPDYR | SEQ ID NO: 322 |
| 324 | PSMB6 | P28072 | Protease | Y59 | TTTGSyIANR | SEQ ID NO: 323 |
| 325 | SENP3 | Q9H4L4 | Protease | S232 | WTPKsPLDPDSGLLSCTLPNGFGGQSGPEGER | SEQ ID NO: 324 |
| 326 | TIF1-beta | Q13263 | Protein kinase | Y458 | QGSGSSQPMEVQEGYGFGSGDDPySSAEPHVSGVKR | SEQ ID NO: 325 |
| 327 | DYRK2 | Q92630 | Protein kinase, | Y309 | VTyIQSR | SEQ ID NO: 326 |
| | | | dual-specificity | | | |
| 328 | Bcr | P11274 | Protein kinase, | Y436 | TGQIWPNDGEGAFHGDADGSFGTPPGyGCAADRAEEQR | SEQ ID NO: 327 |
| | | | Ser/Thr (non- | | | |
| | | | receptor) | | | |
| 329 | Bcr | P11274 | Protein kinase, | Y598 | AFVDNyGVAMEMAEK | SEQ ID NO: 328 |
| | | | Ser/Thr (non- | | | |
| | | | receptor) | | | |
| 330 | Bcr | P11274 | Protein kinase, | Y910 | LQTVHSIPLTINKEDDESPGLyGFLNVIVHSATGFK | SEQ ID NO: 329 |
| | | | Ser/Thr (non- | | | |
| | | | receptor) | | | |
| 331 | CAMKK2 | Q96RR4 | Protein kinase, | S129 | CICPSLPYsPVSSPQSSPRLPR | SEQ ID NO: 330 |
| | | | Ser/Thr (non- | | | |
| | | | receptor) | | | |
| 332 | CAMKK2 | Q96RR4 | Protein kinase, | S133 | CICPSLPYSPVSsPQSSPRLPR | SEQ ID NO: 331 |
| | | | Ser/Thr (non- | | | |
| | | | receptor) | | | |
| 333 | CAMKK2 | Q96RR4 | Protein kinase, | S136 | CICPSLPYSPVSSPQsSPRLPR | SEQ ID NO: 332 |
| | | | Ser/Thr (non- | | | |
| | | | receptor) | | | |
| 334 | CdkL5 | O76039 | Protein kinase, | Y171 | NLSEGNNANYTEyVATR | SEQ ID NO: 333 |
| | | | Ser/Thr (non- | | | |
| | | | receptor) | | | |
| 335 | GRK2 | P25098 | Protein kinase, | Y356 | KKPHASVGTHGyMAPEVLQK | SEQ ID NO: 334 |
| | | | Ser/Thr (non- | | | |
| | | | receptor) | | | |
| 336 | LRRK1 | Q96JN5 | Protein kinase, | Y417 | VTIySFTGNQRNR | SEQ ID NO: 335 |
| | | | Ser/Thr (non- | | | |
| | | | receptor) | | | |
| 337 | MAPKAP | P49137 | Protein kinase, | Y225 | ETTSHNSLTTPCyTPYYVAPEVLGPEK | SEQ ID NO: 336 |
| | K2 | | Ser/Thr (non- | | | |
| | | | receptor) | | | |
| 338 | MAPKAP | P49137 | Protein kinase, | Y228 | ETTSHNSLTTPCYTPyYVAPEVLGPEK | SEQ ID NO: 337 |
| | K2 | | Ser/Thr (non- | | | |
| | | | receptor) | | | |
| 339 | MAPKAP | P49137 | Protein kinase, | Y229 | ETTSHNSLTTPCYTPYyVAPEVLGPEK | SEQ ID NO: 338 |
| | K2 | | Ser/Thr (non- | | | |
| | | | receptor) | | | |
| 340 | MAPKAP | Q16644 | Protein kinase, | Y204 | ETTQNALQTPCyTPYYVAPEVLGPEKYDK | SEQ ID NO: 339 |
| | K3 | | Ser/Thr (non- | | | |
| | | | receptor) | | | |
| 341 | MAPKAP | Q16644 | Protein kinase, | Y207 | ETTQNALQTPCYTPyYVAPEVLGPEKYDK | SEQ ID NO: 340 |
| | K3 | | Ser/Thr (non- | | | |
| | | | receptor) | | | |
| 342 | MAPKAP | Q16644 | Protein kinase, | Y208 | ETTQNALQTPCYTPYyVAPEVLGPEKYDK | SEQ ID NO: 341 |
| | K3 | | Ser/Thr (non- | | | |
| | | | receptor) | | | |
| 343 | MARK2 | Q15524 | Protein kinase, | S585 | DQQNLPYGVTPAsPSGHSQGR | SEQ ID NO: 342 |
| | | | Ser/Thr (non- | | | |
| | | | receptor) | | | |
| 344 | MYO3B | Q8WXR4 | Protein kinase, | Y38 | GTyGKVYKVTNK | SEQ ID NO: 343 |
| | | | Ser/Thr (non- | | | |
| | | | receptor) | | | |
| 345 | PFTAIRE | O94921 | Protein kinase, | Y146 | KADSYEKLEKLGEGSyA | SEQ ID NO: 344 |
| | 1 | | Ser/Thr (non- | | | |
| | | | receptor) | | | |
| 346 | Abl | P00519-2 | Protein kinase, | Y112 | VLGyNHNGEWCEAQTK | SEQ ID NO: 345 |
| | | | tyrosine (non- | | | |
| | | | receptor) | | | |
| 347 | Abl | P00519-2 | Protein kinase, | Y158 | NAAEyLLSSGINGSFLVR | SEQ ID NO. 346 |
| | | | tyrosine (non- | | | |
| | | | receptor) | | | |
| 348 | Abl | P00519-2 | Protein kinase, | Y432 | WTAPESLAyNK | SEQ ID NO: 347 |
| | | | tyrosine (non- | | | |
| | | | receptor) | | | |
| 349 | Arg | P42684 | Protein kinase, | Y161 | SKNGQGWVPSNyITPVNSLEK | SEQ ID NO: 348 |
| | | | tyrosine (non- | | | |
| | | | receptor) | | | |
| 350 | Arg | P42684 | Protein kinase, | Y272 | CNKPTVyGVSPIHDKWEMER | SEQ ID NO: 349 |
| | | | tyrosine (non- | | | |
| | | | receptor) | | | |
| 351 | Arg | P42684 | Protein kinase, | Y303 | HKLGGGQYGEVyVGVWKK | SEQ ID NO: 350 |
| | | | tyrosine (non- | | | |
| | | | receptor) | | | |
| 352 | Arg | P42684 | Protein kinase, | Y310 | YVGVWKKyS | SEQ ID NO: 351 |
| | | | tyrosine (non- | | | |
| | | | receptor) | | | |
| 353 | Arg | P42684 | Protein kinase, | Y568 | AASSSSVVPyLPRLPILPSK | SEQ ID NO: 352 |
| | | | tyrosine (non- | | | |
| | | | receptor) | | | |
| 354 | Arg | P42684 | Protein kinase, | Y683 | SSFREMENQPHKKyE | SEQ ID NO: 353 |
| | | | tyrosine (non- | | | |
| | | | receptor) | | | |
| 355 | Arg | P42684 | Protein kinase, | Y718 | NLVPPKCyGGSFAQRNLCNDDGGGGGGSGTAGGGWSGIT | SEQ ID NO: 354 |
| | | | tyrosine (non- | | G | |
| | | | receptor) | | | |
| 356 | Blk | P51451 | Protein kinase, | Y187 | CLDEGGYyISPR | SEQ ID NO: 355 |
| | | | tyrosine (non- | | | |
| | | | receptor) | | | |
| 357 | Blk | P51451 | Protein kinase, | Y388 | IIDSEyTAQEGAK | SEQ ID NO. 356 |
| | | | tyrosine (non- | | | |
| | | | receptor) | | | |
| 358 | Btk | Q06187 | Protein kinase, | Y225 | KVVALYDyMPMNANDLQLR | SEQ ID NO: 357 |
| | | | tyrosine (non- | | | |
| | | | receptor) | | | |
| 359 | Btk | Q06187 | Protein kinase, | Y361 | HLFSTIPELINyHQHNSAGLISR | SEQ ID NO: 358 |
| | | | tyrosine (non- | | | |
| | | | receptor) | | | |
| 360 | Fgr | P09769 | Protein kinase, | Y28 | SyGAADHYGPDPTK | SEQ ID NO: 359 |
| | | | tyrosine (non- | | | |
| | | | receptor) | | | |
| 361 | Fgr | P09769 | Protein kinase, | Y34 | SYGAADHyGPDPTK | SEQ ID NO: 360 |
| | | | tyrosine (non- | | | |
| | | | receptor) | | | |
| 362 | Fyn | P06241 | Protein kinase, | Y213 | KLDNGGYyITTR | SEQ ID NO: 361 |
| | | | tyrosine (non- | | | |
| | | | receptor) | | | |
| 363 | Lyn | P07948 | Protein kinase, | Y192 | SLDNGGyYISPR | SEQ ID NO: 362 |
| | | | tyrosine (non- | | | |
| | | | receptor) | | | |
| 364 | Lyn | P07948 | Protein kinase, | Y264 | LGAGQFGEVWMGyYNNSTK | SEQ ID NO: 363 |
| | | | tyrosine (non- | | | |
| | | | receptor) | | | |
| 365 | Lyn | P07948 | Protein kinase, | Y31 | TIyVRDPTSNK | SEQ ID NO: 364 |
| | | | tyrosine (non- | | | |
| | | | receptor) | | | |
| 366 | Lyn | P07948 | Protein kinase, | Y472 | VENCPDELyDIMK | SEQ ID NO. 365 |
| | | | tyrosine (non- | | | |
| | | | receptor) | | | |
| 367 | Tyk2 | P29597 | Protein kinase, | Y292 | LLAQAEGEPCyIR | SEQ ID NO: 366 |
| | | | tyrosine (non- | | | |
| | | | receptor) | | | |
| 368 | ZAP70 | P43403 | Protein kinase, | Y397 | EAQIMHQLDNPyIVR | SEQ ID NO: 367 |
| | | | tyrosine (non- | | | |
| | | | receptor) | | | |
| 369 | EphA2 | P29317 | Protein kinase, | Y772 | VLEDDPEATyTTSGGK | SEQ ID NO. 368 |
| | | | tyrosine | | | |
| | | | (receptor) | | | |
| 370 | FLT3 | P36888 | Protein kinase, | Y842 | DIMSDSNyVVR | SEQ ID NO: 369 |
| | | | tyrosine | | | |
| | | | (receptor) | | | |
| 371 | FLT3 | P36888 | Protein kinase, | Y955 | KRPSFPNLTSFLGCQLADAEEAMyQNVDGR | SEQ ID NO: 370 |
| | | | tyrosine | | | |
| | | | (receptor) | | | |
| 372 | FLT3 | P36888 | Protein kinase, | Y969 | VSECPHTyQNR | SEQ ID NO: 371 |
| | | | tyrosine | | | |
| | | | (receptor) | | | |
| 373 | BDP1 | Q99952 | Protein | Y62 | yKDVVAYDETR | SEQ ID NO: 372 |
| | | | phosphatase, | | | |
| | | | tyrosine (non- | | | |
| | | | receptor) | | | |
| 374 | SHP-1 | P29350 | Protein | Y541 | GQESEYGNITyPPAMK | SEQ ID NO. 373 |
| | | | phosphatase, | | | |
| | | | tyrosine (non- | | | |
| | | | receptor) | | | |
| 375 | SHP-1 | P29350 | Protein | Y61 | IQNSGDFyDLYGGEK | SEQ ID NO: 374 |
| | | | phosphatase, | | | |
| | | | tyrosine (non- | | | |
| | | | receptor) | | | |
| 376 | SHP-1 | P29350 | Protein | Y64 | IQNSGDFYDLyGGEK | SEQ ID NO. 375 |
| | | | phosphatase, | | | |
| | | | tyrosine (non- | | | |
| | | | receptor) | | | |
| 377 | PTP- | P23468 | Protein | Y672 | yLLEQLEKWTEYR | SEQ ID NO: 376 |
| | delta | | phosphatase, | | | |
| | | | tyrosine (non- | | | |
| | | | receptor) | | | |
| 378 | PTP- | P23468 | Protein | Y683 | YLLEQLEKWTEyR | SEQ ID NO. 377 |
| | | | phosphatase, | | | |
| | | | tyrosine (non- | | | |
| | | | receptor) | | | |
| 379 | IL-13R | Q14627 | Receptor, | Y73 | yRNIGSETWKTIITK | SEQ ID NO: 378 |
| | alpha 2 | | cytokine | | | |
| 380 | Mpl | P40238 | Receptor, | Y591 | TPLPLCSSQAQMDyR | SEQ ID NO: 379 |
| | | | cytokine | | | |
| 381 | OR2AI1P | XP_068681 | Receptor, GPCR | Y93 | VSyVGCMVQYSVALALGSTECVLLAIMAVDR | SEQ ID NO: 380 |
| 382 | ANTXR1 | Q9H6X2 | Receptor, misc. | Y383 | WPTVDASYyGGR | SEQ ID NO: 381 |
| 383 | KALI | Q96DV0 | Receptor, misc. | Y284 | NLEYVSVSPTNNTVyASVTHSNR | SEQ ID NO: 382 |
| 384 | TyroBP | O43914 | Receptor, misc. | Y102 | SDVySDLNTQRPYYK | SEQ ID NO: 383 |
| 385 | TyroBP | O43914 | Receptor, misc. | Y111 | SDVYSDLNTQRPyYK | SEQ ID NO: 384 |
| 386 | TyroBP | O43914 | Receptor, misc. | Y112 | SDVYSDLNTQRPYyK | SEQ ID NO: 385 |
| 387 | TyroBP | O43914 | Receptor, misc. | Y91 | ITETESPyQELQGQR | SEQ ID NO: 386 |
| 388 | VR1 | Q9NQ74 | Receptor, misc.; | Y310 | FVTSMyNEILILGAK | SEQ ID NO: 387 |
| | | | Channel, cation | | | |
| 389 | PDAP1 | Q13442 | Secreted protein | Y17 | ARQyTSPEEIDAQLQAEKQK | SEQ ID NO: 388 |
| 390 | 4E-BP1 | Q13541 | Translation | Y34 | RVVLGDGVQLPPGDySTTPGGTLFSTTPGGTR | SEQ ID NO: 389 |
| 391 | eEF1A-1 | P04720 | Translation | Y141 | EHALLAyTLGVK | SEQ ID NO: 390 |
| 392 | eEF1A-1 | P04720 | Translation | Y85 | LKAERERGITIDISLWKFETSKyYVTIIDAPGHR | SEQ ID NO: 391 |
| 393 | elF3- | O00303 | Translation | S258 | TCFsPNRVIGLSSDLQQVGGASAR | SEQ ID NO: 392 |
| | epsilon | | | | | |
| 394 | elF3S6IP | Q9Y262 | Translation | Y36 | QDLAyERQYEQQTYQVIPEVIK | SEQ ID NO: 393 |
| 395 | elF3S6IP | Q9Y262 | Translation | Y40 | QDLAYERQyEQQTYQVIPEVIK | SEQ ID NO: 394 |
| 396 | elF3S6IP | Q9Y262 | Translation | Y45 | QDLAYERQYEQQTYQVIPEVIK | SEQ ID NO: 395 |
| 397 | elF4B | P23588 | Translation | Y211 | ARPATDSFDDyPPR | SEQ ID NO: 396 |
| 398 | elF4B | P23588 | Translation | Y316 | DDySRDDYR | SEQ ID NO: 397 |
| 399 | elF4B | P23588 | Translation | Y321 | DDYSRDDyRR | SEQ ID NO: 398 |
| 400 | RPL13A | P40429 | Translation | Y136 | KFAyLGRLAHEVGWKYQAVTATLEEKRK | SEQ ID NO: 399 |
| 401 | RPL13A | P40429 | Translation | Y148 | KFAYLGRLAHEVGWKyQAVTATLEEKRK | SEQ ID NO: 400 |
| 402 | NXT2 | NP_061168 | Transporter | Y23 | SNYyEGPHTSHSSPADR | SEQ ID NO: 401 |
| 403 | RanBP2 | P49792 | Transporter | Y961 | GDDyFNYNVQQTSTNPPLPEPGYFTKPPIAAHASR | SEQ ID NO: 402 |
| 404 | RanBP2 | P49792 | Transporter | Y980 | GDDYFNYNVQQTSTNPPLPEPGyFTKPPIAAHASR | SEQ ID NO: 403 |
| 405 | SLC13A1 | Q9BZW2 | Transporter | Y345 | yQEIVTLVLFIIMALLWFSR | SEQ ID NO: 404 |
| 406 | apollon | Q9NR09 | Ubiquitin | Y2241 | IQSNKGSSyKLLVEQAKLKQATSKHFKDLIR | SEQ ID NO. 405 |
| | | | conjugating | | | |
| | | | system | | | |
| 407 | apollon | Q9NR09 | Ubiquitin | Y4260 | VPNSSVNQTEPQVSSSHNPTSTEEQQLyWAK | SEQ ID NO: 406 |
| | | | conjugating | | | |
| | | | system | | | |
| 408 | Fbx46 | Q6PJ61 | Ubiquitin | S293 | APDSGLPSGGGGRPGCAYPGsPGPGAR | SEQ ID NO: 407 |
| | | | conjugating | | | |
| | | | system | | | |
| 409 | ITCH | Q96J02 | Ubiquitin | Y420 | FIyGNQDLFATSQSK | SEQ ID NO: 408 |
| | | | conjugatin | | | |
| | | | system | | | |
| 410 | RNF26 | Q9BY78 | Ubiquitin | Y432 | RGILQTLNVyL | SEQ ID NO: 409 |
| | | | conjugating | | | |
| | | | system | | | |
| 411 | sequesto- | Q13501 | Ubiquitin | S272 | SRLTPVsPESSSTEEK | SEQ ID NO: 410 |
| | some 1 | | conjugating | | | |
| | | | system | | | |
| 412 | Clathrin | Q00610 | Vesicle protein | Y1487 | TSIDAyDNFDNISLAQR | SEQ ID NO: 411 |
| | heavy | | | | | |
| | chain 1 | | | | | |
| 413 | Clathrin | Q00610 | Vesicle protein | Y634 | GLLQRALEHFTDLyDIKR | SEQ ID NO: 412 |
| | heavy | | | | | |
| | chain 1 | | | | | |
| 414 | COP, | P53618 | Vesicle protein | Y521 | LVTEMGTyATQSALSSSRPTK | SEQ ID NO: 413 |
| | beta | | | | | |
| 415 | HIP14 | BAA76790 | Vesicle protein | Y321 | GyDNPSFLR | SEQ ID NO: 414 |
| 416 | LAPTM5 | Q13571 | Vesicle protein | Y239 | VVLPSyEEALSLPSKTPEGGPAPPPYSEV | SEQ ID NO: 415 |
| 417 | LAPTM5 | Q13571 | Vesicle protein | Y259 | VVLPSYEEALSLPSKTPEGGPAPPPySEV | SEQ ID NO: 416 |
| 418 | neuro- | Q8NFP9 | Vesicle protein | Y253 | WPyQNGFTLNTWFR | SEQ ID NO: 417 |
| | beachin | | | | | |
| 419 | NSFL1C | Q9UNZ2 | Vesicle protein | Y167 | LGAAPEEESAyVAGEKR | SEQ ID NO: 418 |
| 420 | NSFL1C | Q9UNZ2 | Vesicle protein | Y95 | DLIHDQDEDEEEEEGQRFyAGGSER | SEQ ID NO: 419 |
| 421 | SNX18 | Q96RF0 | Vesicle protein | Y274 | LCVVLGPYGPEWQENPyPFQCTIDDPTK | SEQ ID NO: 420 |
| 422 | SNX18 | Q96RF0 | Vesicle protein | Y78 | RyANVPPGGFEPLPV | SEQ ID NO: 421 |
| 423 | TOM1L2 | Q8TDE7 | Vesicle protein | Y160 | TTAGTySSPPPASYSTLQAPALSVTGPITANSEQIAR | SEQ ID NO: 422 |
| 424 | TOM1L2 | Q8TDE7 | Vesicle protein | Y168 | TTAGTYSSPPPASySTLQAPALSVTGPITANSEQIAR | SEQ ID NO: 423 |
| 425 | XRRA1 | Q8NDZ3 | X-radiation | Y666 | NAQALQQMLKHPLLCHSSKPKLDTLQKPyVHK | SEQ ID NO: 424 |
| | | | resistance | | | |

The short name for each protein in which a phosphorylation site has presently been identified is provided in Column A, and its SwissProt accession number (human) is provided Column B. The protein type/group into which each protein falls is provided in Column C. The identified tyrosine or serine residue at which phosphorylation occurs in a given protein is identified in Column D, and the amino acid sequence of the phosphorylation site encompassing the tyrosine residue is provided in Column E (lower case y=the tyrosine, or lower case s=the serine (identified in Column D)) at which phosphorylation occurs. Table 1 above is identical to FIG. 2, except that the latter includes the disease and cell type(s) in which the particular phosphorylation site was identified (Columns F and G).

The identification of these 424 phosphorylation sites is described in more detail in Part A below and in Example 1.

### DEFINITIONS

As used herein, the following terms have the meanings indicated:

“Antibody” or “antibodies” refers to all types of immunoglobulins, including IgG, IgM, IgA, IgD, and IgE, including F_{ab} or antigen-recognition fragments thereof, including chimeric, polyclonal, and monoclonal antibodies. The term “does not bind” with respect to an antibody's binding to one phospho-form of a sequence means does not substantially react with as compared to the antibody's binding to the other phospho-form of the sequence for which the antibody is specific.

“Leukemia-related signaling protein” means any protein (or poly-peptide derived therefrom) enumerated in Column A of Table 1/FIG. 2, which is disclosed herein as being phosphorylated in one or more leukemia cell line(s). Leukemia-related signaling proteins may be tyrosine kinases, such as Flt-3 or BCR-Abl, or serine/threonine kinases, or direct substrates of such kinases, or may be indirect substrates downstream of such kinases in signaling pathways. A Leukemia-related signaling protein may also be phosphorylated in other cell lines (non-leukemic) harboring activated kinase activity.

“Heavy-isotope labeled peptide” (used interchangeably with AQUA peptide) means a peptide comprising at least one heavy-isotope label, which is suitable for absolute quantification or detection of a protein as described in WO/03016861, “Absolute Quantification of Proteins and Modified Forms Thereof by Multistage Mass Spectrometry” (Gygi et al.), further discussed below.

“Protein” is used interchangeably with polypeptide, and includes protein fragments and domains as well as whole protein.

“Phosphorylatable amino acid” means any amino acid that is capable of being modified by addition of a phosphate group, and includes both forms of such amino acid.

“Phosphorylatable peptide sequence” means a peptide sequence comprising a phosphorylatable amino acid.

“Phosphorylation site-specific antibody” means an antibody that specifically binds a phosphorylatable peptide sequence/epitope only when phosphorylated, or only when not phosphorylated, respectively. The term is used interchangeably with “phospho-specific” antibody.

### A. Identification of Novel Leukemia-related Protein Phosphorylation Sites.

The 424 novel Leukemia-related signaling protein phosphorylation sites disclosed herein and listed in Table 1/FIG. 2 were discovered by employing the modified peptide isolation and characterization techniques described in “Immunoaffinity Isolation of Modified Peptides From Complex Mixtures,” U.S. Patent Publication No. 20030044848, Rush et al. (the teaching of which is hereby incorporated herein by reference, in its entirety) using cellular extracts from the following human Leukemia (AML, ALL, CML and CLL) derived cell lines and patient samples: HT-93, KBM-3, SEM, KU-812, SUP-B15, BV-173, CMK, HEL, CLL-220, CLL-1202, CLL23LB4, MEC1, MEC2, M01043, K562, EOL1, HL60, CTV-1, REH, MV4-11, PL-21, and MKPL-1; or from the following cell lines expressing activated BCR-Abl wild type and mutant kinases such as: Baf3-p210 BCR-Abl, Baf3-M351T-BCR-ABL, Baf3-E255K-BCR-Abl, Baf3-Y253F-BCR-Abl, Baf3-T3151-BCR-ABl, 3T3-v-Abl; or activated Flt3 kinase such as Baf3-FLT3. The isolation and identification of phosphopeptides from these cell lines, using an immobilized general phosphotyrosine-specific antibody, or an antibody recognizing the phosphorylated motif PXpSP is described in detail in Example 1 below. In addition to the 424 previously unknown protein phosphorylation sites (tyrosine and serine) discovered, many known phosphorylation sites were also identified (not described herein). The immunoaffinity/mass spectrometric technique described in the '848 patent Publication (the “IAP” method)—and employed as described in detail in the Examples—is briefly summarized below.

The IAP method employed generally comprises the following steps: (a) a proteinaceous preparation (e.g. a digested cell extract) comprising phosphopeptides from two or more different proteins is obtained from an organism; (b) the preparation is contacted with at least one immobilized general phosphotyrosine-specific antibody; (c) at least one phosphopeptide specifically bound by the immobilized antibody in step (b) is isolated; and (d) the modified peptide isolated in step (c) is characterized by mass spectrometry (MS) and/or tandem mass spectrometry (MS-MS). Subsequently, (e) a search program (e.g. Sequest) may be utilized to substantially match the spectra obtained for the isolated, modified peptide during the characterization of step (d) with the spectra for a known peptide sequence. A quantification step employing, e.g. SILAC or AQUA, may also be employed to quantify isolated peptides in order to compare peptide levels in a sample to a baseline.

In the IAP method as employed herein, a general phosphotyrosine-specific monoclonal antibody (commercially available from Cell Signaling Technology, Inc., Beverly, Mass., Cat #9411 (p-Tyr-100)), and an antibody recognizing the phosphorylated motif PxpSP (commercially available from Cell Signaling Technology, Inc., Beverly, Mass., Cat #9325) (pS=phospho-serine) were used in the immunoaffinity step to isolate the widest possible number of phospho-tyrosine and phospho-serine containing peptides from the cell extracts.

Extracts from the following human Leukemia cell lines (ALL, AML, CLL, CML, respectively) were employed: HT-93, KBM-3, SEM, KU-812, SUP-B15, BV-173, CMK, HEL, CLL-220, CLL-1202, CLL23LB4, MEC1, MEC2, MO1043, K562, EOL1, HL60, CTV-1, REH, MV4-11, PL-21, and MKPL-1; or from the following cell lines expressing activated BCR-Abl wild type and mutant kinases such as: Baf3-p210 BCR-Abl, Baf3-M351T-BCR-ABL, Baf3-E255K-BCR-Abl, Baf3-T3151-BCR-ABl, 3T3-v-Abl; or activated Flt3 kinase such as Baf3-FLT3.

As described in more detail in the Examples, lysates were prepared from these cells line and digested with trypsin after treatment with DTT and iodoacetamide to alkylate cysteine residues. Before the immunoaffinity step, peptides were pre-fractionated by reversed-phase solid phase extraction using Sep-Pak C₁₈ columns to separate peptides from other cellular components. The solid phase extraction cartridges were eluted with varying steps of acetonitrile. Each lyophilized peptide fraction was redissolved in PBS and treated with phosphotyrosine or phospho PxpSP antibodies (P-Tyr-100, CST #9411; and 9325, respectively) immobilized on protein G-Sepharose or Protein A-Sepharose. Immunoaffinity-purified peptides were eluted with 0.1% TFA and a portion of this fraction was concentrated with Stage or Zip tips and analyzed by LC-MS/MS, using a ThermoFinnigan LCQ Deca XP Plus ion trap mass spectrometer. Peptides were eluted from a 10 cm×75 μm reversed-phase column with a 45-min linear gradient of acetonitrile. MS/MS spectra were evaluated using the program Sequest with the NCBI human protein database.

This revealed a total of 424 novel tyrosine or serine phosphorylation sites in signaling pathways affected by kinase activation or active in leukemia cells. The identified phosphorylation sites and their parent proteins are enumerated in Table 1/FIG. 2. The tyrosine or serine (human sequence) at which phosphorylation occurs is provided in Column D, and the peptide sequence encompassing the phosphorylatable tyrosine or serine residue at the site is provided in Column E. FIG. 2 also shows the particular type of leukemic disease (see Column G) and cell line(s) (see Column F) in which a particular phosphorylation site was discovered.

As a result of the discovery of these phosphorylation sites, phospho-specific antibodies and AQUA peptides for the detection of and quantification of these sites and their parent proteins may now be produced by standard methods, described below. These new reagents will prove highly useful in, e.g., studying the signaling pathways and events underlying the progression of leukemias and the identification of new biomarkers and targets for diagnosis and treatment of such diseases.

### B. Antibodies and Cell Lines

Isolated phosphorylation site-specific antibodies that specifically bind a Leukemia-related signaling protein disclosed in Column A of Table 1 only when phosphorylated (or only when not phosphorylated) at the corresponding amino acid and phosphorylation site listed in Columns D and E of Table 1/FIG. 2 may now be produced by standard antibody production methods, such as anti-peptide antibody methods, using the phosphorylation site sequence information provided in Column E of Table 1. For example, two previously unknown Blk kinase phosphorylation sites (tyrosines 187 and 388) (see Rows 356-357 of Table 1/FIG. 2) are presently disclosed. Thus, antibodies that specifically bind either of these novel Blk kinase sites can now be produced, e.g. by immunizing an animal with a peptide antigen comprising all or part of the amino acid sequence encompassing the respective phosphorylated residue (e.g. a peptide antigen comprising the sequence set forth in Row 357, Column E, of Table 1 (SEQ ID NO: 356) (which encompasses the phosphorylated tyrosine at position 388 in Blk), to produce an antibody that only binds Blk kinase when phosphorylated at that site.

Polyclonal antibodies of the invention may be produced according to standard techniques by immunizing a suitable animal (e.g., rabbit, goat, etc.) with a peptide antigen corresponding to the Leukemia-related phosphorylation site of interest (i.e. a phosphorylation site enumerated in Column E of Table 1, which comprises the corresponding phosphorylatable amino acid listed in Column D of Table 1), collecting immune serum from the animal, and separating the polyclonal antibodies from the immune serum, in accordance with known procedures. For example, a peptide antigen corresponding to all or part of the novel MARK2 kinase phosphorylation site disclosed herein (SEQ ID NO: 342=DQQNLPYGVTPAsPSGHSQGR, encompassing phosphorylated serine 585 (see Row 343 of Table 1)) may be used to produce antibodies that only bind MARK2 when phosphorylated at Ser585. Similarly, a peptide comprising all or part of any one of the phosphorylation site sequences provided in Column E of Table 1 may employed as an antigen to produce an antibody that only binds the corresponding protein listed in Column A of Table 1 when phosphorylated (or when not phosphorylated) at the corresponding residue listed in Column D. If an antibody that only binds the protein when phosphorylated at the disclosed site is desired, the peptide antigen includes the phosphorylated form of the amino acid. Conversely, if an antibody that only binds the protein when not phosphorylated at the disclosed site is desired, the peptide antigen includes the non-phosphorylated form of the amino acid.

Peptide antigens suitable for producing antibodies of the invention may be designed, constructed and employed in accordance with well-known techniques. See, e.g., ANTIBODIES: A LABORATORY MANUAL, Chapter 5, p. 75-76, Harlow & Lane Eds., Cold Spring Harbor Laboratory (1988); Czernik, Methods In Enzymology, 201: 264-283 (1991); Merrifield, J. Am. Chem. Soc. 85:21-49 (1962)).

It will be appreciated by those of skill in the art that longer or shorter phosphopeptide antigens may be employed. See Id. For example, a peptide antigen may comprise the full sequence disclosed in Column E of Table 1/FIG. 2, or it may comprise additional amino acids flanking such disclosed sequence, or may comprise of only a portion of the disclosed sequence immediately flanking the phosphorylatable amino acid (indicated in Column E by lowercase “y” or “s”). Typically, a desirable peptide antigen will comprise four or more amino acids flanking each side of the phosphorylatable amino acid and encompassing it. Polyclonal antibodies produced as described herein may be screened as further described below.

Monoclonal antibodies of the invention may be produced in a hybridoma cell line according to the well-known technique of Kohler and Milstein. See Nature 265:495-97 (1975); Kohler and Milstein, Eur. J. Immunol. 6: 511 (1976); see also, CURRENT PROTOCOLS IN MOLECULAR BIOLOGY, Ausubel et al. Eds. (1989). Monoclonal antibodies so produced are highly specific, and improve the selectivity and specificity of diagnostic assay methods provided by the invention. For example, a solution containing the appropriate antigen may be injected into a mouse or other species and, after a sufficient time (in keeping with conventional techniques), the animal is sacrificed and spleen cells obtained. The spleen cells are then immortalized by fusing them with myeloma cells, typically in the presence of polyethylene glycol, to produce hybridoma cells. Rabbit fusion hybridomas, for example, may be produced as described in U.S. Pat. No. 5,675,063, C. Knight, Issued Oct. 7, 1997. The hybridoma cells are then grown in a suitable selection media, such as hypoxanthine-aminopterin-thymidine (HAT), and the supernatant screened for monoclonal antibodies having the desired specificity, as described below. The secreted antibody may be recovered from tissue culture supernatant by conventional methods such as precipitation, ion exchange or affinity chromatography, or the like.

Monoclonal Fab fragments may also be produced in Escherichia coli by recombinant techniques known to those skilled in the art. See, e.g., W. Huse, Science 246:1275-81 (1989); Mullinax et al., Proc. Nat'l Acad. Sci. 87: 8095 (1990). If monoclonal antibodies of one isotype are preferred for a particular application, particular isotypes can be prepared directly, by selecting from the initial fusion, or prepared secondarily, from a parental hybridoma secreting a monoclonal antibody of different isotype by using the sib selection technique to isolate class-switch variants (Steplewski, et al., Proc. Nat'l. Acad. Sci., 82: 8653 (1985); Spira et al., J. Immunol. Methods, 74: 307 (1984)).

The preferred epitope of a phosphorylation-site specific antibody of the invention is a peptide fragment consisting essentially of about 8 to 17 amino acids including the phosphorylatable tyrosine or serine, wherein about 3 to 8 amino acids are positioned on each side of the phosphorylatable tyrosine (for example, the BCAP tyrosine 392 phosphorylation site sequence disclosed in Row 8, Column E of Table 1), and antibodies of the invention thus specifically bind a target Leukemia-related signaling polypeptide comprising such epitopic sequence. Particularly preferred epitopes bound by the antibodies of the invention comprise all or part of a phosphorylatable site sequence listed in Column E of Table 1, including the phosphorylatable amino acid.

Included in the scope of the invention are equivalent non-antibody molecules, such as protein binding domains or nucleic acid aptamers, which bind, in a phospho-specific manner, to essentially the same phosphorylatable epitope to which the phospho-specific antibodies of the invention bind. See, e.g., Neuberger et al., Nature 312: 604 (1984). Such equivalent non-antibody reagents may be suitably employed in the methods of the invention further described below.

Antibodies provided by the invention may be any type of immunoglobulins, including IgG, IgM, IgA, IgD, and IgE, including F_{ab} or antigen-recognition fragments thereof. The antibodies may be monoclonal or polyclonal and may be of any species of origin, including (for example) mouse, rat, rabbit, horse, or human, or may be chimeric antibodies. See, e.g., M. Walker et al., Molec. Immunol. 26: 403-11 (1989); Morrision et al., Proc. Nat'l. Acad. Sci. 81: 6851 (1984); Neuberger et al., Nature 312: 604 (1984)). The antibodies may be recombinant monoclonal antibodies produced according to the methods disclosed in U.S. Pat. No. 4,474,893 (Reading) or U.S. Pat. No. 4,816,567 (Cabilly et al.) The antibodies may also be chemically constructed by specific antibodies made according to the method disclosed in U.S. Pat. No. 4,676,980 (Segel et al.)

The invention also provides immortalized cell lines that produce an antibody of the invention. For example, hybridoma clones, constructed as described above, that produce monoclonal antibodies to the Leukemia-related signaling protein phosphorylation sties disclosed herein are also provided. Similarly, the invention includes recombinant cells producing an antibody of the invention, which cells may be constructed by well known techniques; for example the antigen combining site of the monoclonal antibody can be cloned by PCR and single-chain antibodies produced as phage-displayed recombinant antibodies or soluble antibodies in E. coli (see, e.g., ANTIBODY ENGINEERING PROTOCOLS, 1995, Humana Press, Sudhir Paul editor.)

Phosphorylation site-specific antibodies of the invention, whether polyclonal or monoclonal, may be screened for epitope and phospho-specificity according to standard techniques. See, e.g. Czemik et al., Methods in Enzymology, 201: 264-283 (1991). For example, the antibodies may be screened against the phospho and non-phospho peptide library by ELISA to ensure specificity for both the desired antigen (i.e. that epitope including a phosphorylation site sequence enumerated in Column E of Table 1) and for reactivity only with the phosphorylated (or non-phosphorylated) form of the antigen. Peptide competition assays may be carried out to confirm lack of reactivity with other phospho-epitopes on the given Leukemia-related signaling protein. The antibodies may also be tested by Western blotting against cell preparations containing the signaling protein, e.g. cell lines over-expressing the target protein, to confirm reactivity with the desired phosphorylated epitope/target.

Specificity against the desired phosphorylated epitope may also be examined by constructing mutants lacking phosphorylatable residues at positions outside the desired epitope that are known to be phosphorylated, or by mutating the desired phospho-epitope and confirming lack of reactivity. Phosphorylation-site specific antibodies of the invention may exhibit some limited cross-reactivity to related epitopes in non-target proteins. This is not unexpected as most antibodies exhibit some degree of cross-reactivity, and anti-peptide antibodies will often cross-react with epitopes having high homology to the immunizing peptide. See, e.g., Czernik, supra. Cross-reactivity with non-target proteins is readily characterized by Western blotting alongside markers of known molecular weight. Amino acid sequences of cross-reacting proteins may be examined to identify sites highly homologous to the Leukemia-related signaling protein epitope for which the antibody of the invention is specific.

In certain cases, polyclonal antisera may exhibit some undesirable general cross-reactivity to phosphotyrosine or phosphoserine itself, which may be removed by further purification of antisera, e.g. over a phosphotyramine column. Antibodies of the invention specifically bind their target protein (i.e. a protein listed in Column A of Table 1) only when phosphorylated (or only when not phosphorylated, as the case may be) at the site disclosed in corresponding Columns D/E, and do not (substantially) bind to the other form (as compared to the form for which the antibody is specific).

Antibodies may be further characterized via immunohistochemical (1HC) staining using normal and diseased tissues to examine Leukemia-related phosphorylation and activation status in diseased tissue. IHC may be carried out according to well-known techniques. See, e.g., ANTIBODIES: A LABORATORY MANUAL, Chapter 10, Harlow & Lane Eds., Cold Spring Harbor Laboratory (1988). Briefly, paraffin-embedded tissue (e.g. tumor tissue) is prepared for immunohistochemical staining by deparaffinizing tissue sections with xylene followed by ethanol; hydrating in water then PBS; unmasking antigen by heating slide in sodium citrate buffer; incubating sections in hydrogen peroxide; blocking in blocking solution; incubating slide in primary antibody and secondary antibody; and finally detecting using ABC avidin/biotin method according to manufacturer's instructions.

Antibodies may be further characterized by flow cytometry carried out according to standard methods. See Chow et al., Cytometry (Communications in Clinical Cytometry) 46: 72-78 (2001). Briefly and by way of example, the following protocol for cytometric analysis may be employed: samples may be centrifuged on Ficoll gradients to remove erythrocytes, and cells may then be fixed with 2% paraformaldehyde for 10 minutes at 37° C. followed by permeabilization in 90% methanol for 30 minutes on ice. Cells may then be stained with the primary phosphorylation-site specific antibody of the invention (which detects a Leukemia-related signal transduction protein enumerated in Table 1), washed and labeled with a fluorescent-labeled secondary antibody. Additional fluorochrome-conjugated marker antibodies (e.g. CD45, CD34) may also be added at this time to aid in the subsequent identification of specific hematopoietic cell types. The cells would then be analyzed on a flow cytometer (e.g. a Beckman Coulter FC500) according to the specific protocols of the instrument used.

Antibodies of the invention may also be advantageously conjugated to fluorescent dyes (e.g. Alexa488, PE) for use in multi-parametric analyses along with other signal transduction (phospho-CrkL, phospho-Erk 1/2) and/or cell marker (CD34) antibodies.

Phosphorylation-site specific antibodies of the invention specifically bind to a human Leukemia-related signal transduction protein or polypeptide only when phosphorylated at a disclosed site, but are not limited only to binding the human species, perse. The invention includes antibodies that also bind conserved and highly homologous or identical phosphorylation sites in respective Leukemia-related proteins from other species (e.g. mouse, rat, monkey, yeast), in addition to binding the human phosphorylation site. Highly homologous or identical sites conserved in other species can readily be identified by standard sequence comparisons, such as using BLAST, with the human Leukemia-related signal transduction protein phosphorylation sites disclosed herein.

### C. Heavy-isotope Labeled Peptides (AQUA Peptides).

The novel Leukemia-related signaling protein phosphorylation sites disclosed herein now enable the production of corresponding heavy-isotope labeled peptides for the absolute quantification of such signaling proteins (both phosphorylated and not phosphorylated at a disclosed site) in biological samples. The production and use of AQUA peptides for the absolute quantification of proteins (AQUA) in complex mixtures has been described. See WO/03016861, “Absolute Quantification of Proteins and Modified Forms Thereof by Multistage Mass Spectrometry,” Gygi et al. and also Gerber et al. Proc. Natl. Acad. Sci. U.S.A. 100: 6940-5 (2003) (the teachings of which are hereby incorporated herein by reference, in their entirety).

The AQUA methodology employs the introduction of a known quantity of at least one heavy-isotope labeled peptide standard (which has a unique signature detectable by LC-SRM chromatography) into a digested biological sample in order to determine, by comparison to the peptide standard, the absolute quantity of a peptide with the same sequence and protein modification in the biological sample. Briefly, the AQUA methodology has two stages: peptide internal standard selection and validation and method development; and implementation using validated peptide internal standards to detect and quantify a target protein in sample. The method is a powerful technique for detecting and quantifying a given peptide/protein within a complex biological mixture, such as a cell lysate, and may be employed, e.g., to quantify change in protein phosphorylation as a result of drug treatment, or to quantify differences in the level of a protein in different biological states.

Generally, to develop a suitable internal standard, a particular peptide (or modified peptide) within a target protein sequence is chosen based on its amino acid sequence and the particular protease to be used to digest. The peptide is then generated by solid-phase peptide synthesis such that one residue is replaced with that same residue containing stable isotopes (¹³C, ¹⁵N). The result is a peptide that is chemically identical to its native counterpart formed by proteolysis, but is easily distinguishable by MS via a 7-Da mass shift. A newly synthesized AQUA internal standard peptide is then evaluated by LC-MS/MS. This process provides qualitative information about peptide retention by reverse-phase chromatography, ionization efficiency, and fragmentation via collision-induced dissociation. Informative and abundant fragment ions for sets of native and internal standard peptides are chosen and then specifically monitored in rapid succession as a function of chromatographic retention to form a selected reaction monitoring (LC-SRM) method based on the unique profile of the peptide standard.

The second stage of the AQUA strategy is its implementation to measure the amount of a protein or modified protein from complex mixtures. Whole cell lysates are typically fractionated by SDS-PAGE gel electrophoresis, and regions of the gel consistent with protein migration are excised. This process is followed by in-gel proteolysis in the presence of the AQUA peptides and LC-SRM analysis. (See Gerber et al. supra.) AQUA peptides are spiked in to the complex peptide mixture obtained by digestion of the whole cell lysate with a proteolytic enzyme and subjected to immunoaffinity purification as described above. The retention time and fragmentation pattern of the native peptide formed by digestion (e.g. trypsinization) is identical to that of the AQUA internal standard peptide determined previously; thus, LC-MS/MS analysis using an SRM experiment results in the highly specific and sensitive measurement of both internal standard and analyte directly from extremely complex peptide mixtures. Because an absolute amount of the AQUA peptide is added (e.g. 250 fmol), the ratio of the areas under the curve can be used to determine the precise expression levels of a protein or phosphorylated form of a protein in the original cell lysate. In addition, the internal standard is present during in-gel digestion as native peptides are formed, such that peptide extraction efficiency from gel pieces, absolute losses during sample handling (including vacuum centrifugation), and variability during introduction into the LC-MS system do not affect the determined ratio of native and AQUA peptide abundances.

An AQUA peptide standard is developed for a known phosphorylation site sequence previously identified by the IAP-LC-MS/MS method within a target protein. One AQUA peptide incorporating the phosphorylated form of the particular residue within the site may be developed, and a second AQUA peptide incorporating the non-phosphorylated form of the residue developed. In this way, the two standards may be used to detect and quantify both the phosphorylated and non-phosphorylated forms of the site in a biological sample.

Peptide internal standards may also be generated by examining the primary amino acid sequence of a protein and determining the boundaries of peptides produced by protease cleavage. Alternatively, a protein may actually be digested with a protease and a particular peptide fragment produced can then sequenced. Suitable proteases include, but are not limited to, serine proteases (e.g. trypsin, hepsin), metallo proteases (e.g. PUMP1), chymotrypsin, cathepsin, pepsin, thermolysin, carboxypeptidases, etc.

A peptide sequence within a target protein is selected according to one or more criteria to optimize the use of the peptide as an internal standard. Preferably, the size of the peptide is selected to minimize the chances that the peptide sequence will be repeated elsewhere in other non-target proteins. Thus, a peptide is preferably at least about 6 amino acids. The size of the peptide is also optimized to maximize ionization frequency. Thus, peptides longer than about 20 amino acids are not preferred. The preferred ranged is about 7 to 15 amino acids. A peptide sequence is also selected that is not likely to be chemically reactive during mass spectrometry, thus sequences comprising cysteine, tryptophan, or methionine are avoided.

A peptide sequence that does not include a modified region of the target region may be selected so that the peptide internal standard can be used to determine the quantity of all forms of the protein. Alternatively, a peptide internal standard encompassing a modified amino acid may be desirable to detect and quantify only the modified form of the target protein. Peptide standards for both modified and unmodified regions can be used together, to determine the extent of a modification in a particular sample (i.e. to determine what fraction of the total amount of protein is represented by the modified form). For example, peptide standards for both the phosphorylated and unphosphorylated form of a protein known to be phosphorylated at a particular site can be used to quantify the amount of phosphorylated form in a sample.

The peptide is labeled using one or more labeled amino acids (i.e. the label is an actual part of the peptide) or less preferably, labels may be attached after synthesis according to standard methods. Preferably, the label is a mass-altering label selected based on the following considerations: The mass should be unique to shift fragment masses produced by MS analysis to regions of the spectrum with low background; the ion mass signature component is the portion of the labeling moiety that preferably exhibits a unique ion mass signature in MS analysis; the sum of the masses of the constituent atoms of the label is preferably uniquely different than the fragments of all the possible amino acids. As a result, the labeled amino acids and peptides are readily distinguished from unlabeled ones by the ion/mass pattern in the resulting mass spectrum. Preferably, the ion mass signature component imparts a mass to a protein fragment that does not match the residue mass for any of the natural amino acids.

The label should be robust under the fragmentation conditions of MS and not undergo unfavorable fragmentation. Labeling chemistry should be efficient under a range of conditions, particularly denaturing conditions, and the labeled tag preferably remains soluble in the MS buffer system of choice. The label preferably does not suppress the ionization efficiency of the protein and is not chemically reactive. The label may contain a mixture of two or more isotopically distinct species to generate a unique mass spectrometric pattern at each labeled fragment position. Stable isotopes, such as ²H, ¹³C, ¹⁵N, ¹⁷O, ¹⁸O, or ³⁴S, are among preferred labels. Pairs of peptide internal standards that incorporate a different isotope label may also be prepared. Preferred amino acid residues into which a heavy isotope label may be incorporated include leucine, proline, valine, and phenylalanine.

Peptide internal standards are characterized according to their mass-to-charge (m/z) ratio, and preferably, also according to their retention time on a chromatographic column (e.g. an HPLC column). Internal standards that co-elute with unlabeled peptides of identical sequence are selected as optimal internal standards. The internal standard is then analyzed by fragmenting the peptide by any suitable means, for example by collision-induced dissociation (CID) using, e.g., argon or helium as a collision gas. The fragments are then analyzed, for example by multi-stage mass spectrometry (MSⁿ) to obtain a fragment ion spectrum, to obtain a peptide fragmentation signature. Preferably, peptide fragments have significant differences in m/z ratios to enable peaks corresponding to each fragment to be well separated, and a signature that is unique for the target peptide is obtained. If a suitable fragment signature is not obtained at the first stage, additional stages of MS are performed until a unique signature is obtained.

Fragment ions in the MS/MS and MS³ spectra are typically highly specific for the peptide of interest, and, in conjunction with LC methods, allow a highly selective means of detecting and quantifying a target peptide/protein in a complex protein mixture, such as a cell lysate, containing many thousands or tens of thousands of proteins. Any biological sample potentially containing a target protein/peptide of interest may be assayed. Crude or partially purified cell extracts are preferably employed. Generally, the sample has at least 0.01 mg of protein, typically a concentration of 0.1-10 mg/mL, and may be adjusted to a desired buffer concentration and pH.

A known amount of a labeled peptide internal standard, preferably about 10 femtomoles, corresponding to a target protein to be detected/quantified is then added to a biological sample, such as a cell lysate. The spiked sample is then digested with one or more protease(s) for a suitable time period to allow digestion. A separation is then performed (e.g. by HPLC, reverse-phase HPLC, capillary electrophoresis, ion exchange chromatography, etc.) to isolate the labeled internal standard and its corresponding target peptide from other peptides in the sample. Microcapillary LC is a preferred method.

Each isolated peptide is then examined by monitoring of a selected reaction in the MS. This involves using the prior knowledge gained by the characterization of the peptide internal standard and then requiring the MS to continuously monitor a specific ion in the MS/MS or MSⁿ spectrum for both the peptide of interest and the internal standard. After elution, the area under the curve (AUC) for both peptide standard and target peptide peaks are calculated. The ratio of the two areas provides the absolute quantification that can be normalized for the number of cells used in the analysis and the protein's molecular weight, to provide the precise number of copies of the protein per cell. Further details of the AQUA methodology are described in Gygi et al., and Gerber et al. supra.

In accordance with the present invention, AQUA internal peptide standards (heavy-isotope labeled peptides) may now be produced, as described above, for any of the 424 novel Leukemia-related signaling protein phosphorylation sites disclosed herein (see Table 1/FIG. 2). Peptide standards for a given phosphorylation site (e.g. the tyrosine 199 in Talin 1—see Row 142 of Table 1) may be produced for both the phosphorylated and non-phosphorylated forms of the site (e.g. see Talin 1 site sequence in Column E, Row 142 of Table 1 (SEQ ID NO: 141) and such standards employed in the AQUA methodology to detect and quantify both forms of such phosphorylation site in a biological sample.

AQUA peptides of the invention may comprise all, or part of, a phosphorylation site peptide sequence disclosed herein (see Column E of Table 1/FIG. 2). In a preferred embodiment, an AQUA peptide of the invention comprises a phosphorylation site sequence disclosed herein in Table 1/FIG. 2. For example, an AQUA peptide of the invention for detection/quantification of Bcr kinase when phosphorylated at tyrosine Y598 may comprise the sequence AFVDNyGVAMEMAEK (y=phosphotyrosine), which comprises phosphorylatable tyrosine 598 (see Row 329, Column E; (SEQ ID NO: 328)). Heavy-isotope labeled equivalents of the peptides enumerated in Table 1/FIG. 2 (both in phosphorylated and unphosphorylated form) can be readily synthesized and their unique MS and LC-SRM signature determined, so that the peptides are validated as AQUA peptides and ready for use in quantification experiments.

The phosphorylation site peptide sequences disclosed herein (see Column E of Table 1/FIG. 2) are particularly well suited for development of corresponding AQUA peptides, since the IAP method by which they were identified (see Part A above and Example 1) inherently confirmed that such peptides are in fact produced by enzymatic digestion (trypsinization) and are in fact suitably fractionated/ionized in MS/MS. Thus, heavy-isotope labeled equivalents of these peptides (both in phosphorylated and unphosphorylated form) can be readily synthesized and their unique MS and LC-SRM signature determined, so that the peptides are validated as AQUA peptides and ready for use in quantification experiments.

Accordingly, the invention provides heavy-isotope labeled peptides (AQUA peptides) for the detection and/or quantification of any of the Leukemia-related phosphorylation sites disclosed in Table 1/FIG. 2 (see Column E) and/or their corresponding parent proteins/polypeptides (see Column A). A phosphopeptide sequence comprising any of the phosphorylation sequences listed in Table 1 may be considered a preferred AQUA peptide of the invention. For example, an AQUA peptide comprising the sequence VENCPDELyDIMK (SEQ ID NO: 365) (where y may be either phosphotyrosine or tyrosine, and where V=labeled valine (e.g. ¹⁴C)) is provided for the quantification of phosphorylated (or non-phosphorylated) Lyn kinase (Tyr472) in a biological sample (see Row 366 of Table 1, tyrosine 472 being the phosphorylatable residue within the site). However, it will be appreciated that a larger AQUA peptide comprising a disclosed phosphorylation site sequence (and additional residues downstream or upstream of it) may also be constructed. Similarly, a smaller AQUA peptide comprising less than all of the residues of a disclosed phosphorylation site sequence (but still comprising the phosphorylatable residue enumerated in Column D of Table 1/FIG. 2) may alternatively be constructed. Such larger or shorter AQUA peptides are within the scope of the present invention, and the selection and production of preferred AQUA peptides may be carried out as described above (see Gygi et al., Gerber et al. supra.).

Certain particularly preferred subsets of AQUA peptides provided by the invention are described above (corresponding to particular protein types/groups in Table 1, for example, Tyrosine Protein Kinases or Protein Phosphatases). Example 4 is provided to further illustrate the construction and use, by standard methods described above, of exemplary AQUA peptides provided by the invention. For example, the above-described AQUA peptides corresponding to the both the phosphorylated and non-phosphorylated forms of the disclosed Lyn kinase tyrosine 472 phosphorylation site (see Row 366 of Table 1/FIG. 2) may be used to quantify the amount of phosphorylated Lyn(Tyr472) in a biological sample, e.g. a tumor cell sample (or a sample before or after treatment with a test drug).

AQUA peptides of the invention may also be employed within a kit that comprises one or multiple AQUA peptide(s) provided herein (for the quantification of a Leukemia-related signal transduction protein disclosed in Table 1/FIG. 2), and, optionally, a second detecting reagent conjugated to a detectable group. For example, a kit may include AQUA peptides for both the phosphorylated and non-phosphorylated form of a phosphorylation site disclosed herein. The reagents may also include ancillary agents such as buffering agents and protein stabilizing agents, e.g., polysaccharides and the like. The kit may further include, where necessary, other members of the signal-producing system of which system the detectable group is a member (e.g., enzyme substrates), agents for reducing background interference in a test, control reagents, apparatus for conducting a test, and the like. The test kit may be packaged in any suitable manner, typically with all elements in a single container along with a sheet of printed instructions for carrying out the test.

AQUA peptides provided by the invention will be highly useful in the further study of signal transduction anomalies underlying cancer, including leukemias, and in identifying diagnostic/bio-markers of these diseases, new potential drug targets, and/or in monitoring the effects of test compounds on Leukemia-related signal transduction proteins and pathways.

### D. Immunoassay Formats

Antibodies provided by the invention may be advantageously employed in a variety of standard immunological assays (the use of AQUA peptides provided by the invention is described separately above). Assays may be homogeneous assays or heterogeneous assays. In a homogeneous assay the immunological reaction usually involves a phosphorylation-site specific antibody of the invention), a labeled analyte, and the sample of interest. The signal arising from the label is modified, directly or indirectly, upon the binding of the antibody to the labeled analyte. Both the immunological reaction and detection of the extent thereof are carried out in a homogeneous solution. Immunochemical labels that may be employed include free radicals, radioisotopes, fluorescent dyes, enzymes, bacteriophages, coenzymes, and so forth.

In a heterogeneous assay approach, the reagents are usually the specimen, a phosphorylation-site specific antibody of the invention, and suitable means for producing a detectable signal. Similar specimens as described above may be used. The antibody is generally immobilized on a support, such as a bead, plate or slide, and contacted with the specimen suspected of containing the antigen in a liquid phase. The support is then separated from the liquid phase and either the support phase or the liquid phase is examined for a detectable signal employing means for producing such signal. The signal is related to the presence of the analyte in the specimen. Means for producing a detectable signal include the use of radioactive labels, fluorescent labels, enzyme labels, and so forth. For example, if the antigen to be detected contains a second binding site, an antibody which binds to that site can be conjugated to a detectable group and added to the liquid phase reaction solution before the separation step. The presence of the detectable group on the solid support indicates the presence of the antigen in the test sample. Examples of suitable immunoassays are the radioimmunoassay, immunofluorescence methods, enzyme-linked immunoassays, and the like.

Immunoassay formats and variations thereof that may be useful for carrying out the methods disclosed herein are well known in the art. See generally E. Maggio, Enzyme-Immunoassay, (1980) (CRC Press, Inc., Boca Raton, Fla.); see also, e.g., U.S. Pat. No. 4,727,022 (Skold et al., “Methods for Modulating Ligand-Receptor Interactions and their Application”); U.S. Pat. No. 4,659,678 (Forrest et al., “Immunoassay of Antigens”); U.S. Pat. No. 4,376,110 (David et al., “Immunometric Assays Using Monoclonal Antibodies”). Conditions suitable for the formation of reagent-antibody complexes are well described. See id. Monoclonal antibodies of the invention may be used in a “two-site” or “sandwich” assay, with a single cell line serving as a source for both the labeled monoclonal antibody and the bound monoclonal antibody. Such assays are described in U.S. Pat. No. 4,376,110. The concentration of detectable reagent should be sufficient such that the binding of a target Leukemia-related signal transduction protein is detectable compared to background.

Phosphorylation site-specific antibodies disclosed herein may be conjugated to a solid support suitable for a diagnostic assay (e.g., beads, plates, slides or wells formed from materials such as latex or polystyrene) in accordance with known techniques, such as precipitation. Antibodies, or other target protein or target site-binding reagents, may likewise be conjugated to detectable groups such as radiolabels (e.g., ³⁵S, ¹²⁵I, ¹³¹I), enzyme labels (e.g., horseradish peroxidase, alkaline phosphatase), and fluorescent labels (e.g., fluorescein) in accordance with known techniques.

Antibodies of the invention may also be optimized for use in a flow cytometry (FC) assay to determine the activation/phosphorylation status of a target Leukemia-related signal transduction protein in patients before, during, and after treatment with a drug targeted at inhibiting phosphorylation at such a protein at the phosphorylation site disclosed herein. For example, bone marrow cells or peripheral blood cells from patients may be analyzed by flow cytometry for target Leukemia-related signal transduction protein phosphorylation, as well as for markers identifying various hematopoietic cell types. In this manner, activation status of the malignant cells may be specifically characterized. Flow cytometry may be carried out according to standard methods. See, e.g. Chow et al., Cytometry (Communications in Clinical Cytometry) 46:72-78 (2001). Briefly and by way of example, the following protocol for cytometric analysis may be employed: fixation of the cells with 1% para-formaldehyde for 10 minutes at 37° C. followed by permeabilization in 90% methanol for 30 minutes on ice. Cells may then be stained with the primary antibody (a phospho-specific antibody of the invention), washed and labeled with a fluorescent-labeled secondary antibody. Alternatively, the cells may be stained with a fluorescent-labeled primary antibody. The cells would then be analyzed on a flow cytometer (e.g. a Beckman Coulter EPICS-XL) according to the specific protocols of the instrument used. Such an analysis would identify the presence of activated Leukemia-related signal transduction protein(s) in the malignant cells and reveal the drug response on the targeted protein.

Alternatively, antibodies of the invention may be employed in immunohistochemical (1HC) staining to detect differences in signal transduction or protein activity using normal and diseased tissues. IHC may be carried out according to well-known techniques. See, e.g., ANTIBODIES: A LABORATORY MANUAL, supra. Briefly, paraffin-embedded tissue (e.g. tumor tissue) is prepared for immunohistochemical staining by deparaffinizing tissue sections with xylene followed by ethanol; hydrating in water then PBS; unmasking antigen by heating slide in sodium citrate buffer; incubating sections in hydrogen peroxide; blocking in blocking solution; incubating slide in primary antibody and secondary antibody; and finally detecting using ABC avidin/biotin method according to manufacturer's instructions.

Antibodies of the invention may be also be optimized for use in other clinically-suitable applications, for example bead-based multiplex-type assays, such as IGEN, Luminex™ and/or Bioplex™ assay formats, or otherwise optimized for antibody arrays formats, such as reversed-phase array applications (see, e.g. Paweletz et al., Oncogene 20(16): 1981-89 (2001)). Accordingly, in another embodiment, the invention provides a method for the multiplex detection of Leukemia-related protein phosphorylation in a biological sample, the method comprising utilizing two or more antibodies or AQUA peptides of the invention to detect the presence of two or more phosphorylated Leukemia-related signaling proteins enumerated in Column A of Table 1/FIG. 2. In one preferred embodiment, two to five antibodies or AQUA peptides of the invention are employed in the method. In another preferred embodiment, six to ten antibodies or AQUA peptides of the invention are employed, while in another preferred embodiment eleven to twenty such reagents are employed.

Antibodies and/or AQUA peptides of the invention may also be employed within a kit that comprises at least one phosphorylation site-specific antibody or AQUA peptide of the invention (which binds to or detects a Leukemia-related signal transduction protein disclosed in Table 1/FIG. 2), and, optionally, a second antibody conjugated to a detectable group. In some embodies, the kit is suitable for multiplex assays and comprises two or more antibodies or AQUA peptides of the invention, and in some embodiments, comprises two to five, six to ten, or eleven to twenty reagents of the invention. The kit may also include ancillary agents such as buffering agents and protein stabilizing agents, e.g., polysaccharides and the like. The kit may further include, where necessary, other members of the signal-producing system of which system the detectable group is a member (e.g., enzyme substrates), agents for reducing background interference in a test, control reagents, apparatus for conducting a test, and the like. The test kit may be packaged in any suitable manner, typically with all elements in a single container along with a sheet of printed instructions for carrying out the test.

The following Examples are provided only to further illustrate the invention, and are not intended to limit its scope, except as provided in the claims appended hereto. The present invention encompasses modifications and variations of the methods taught herein which would be obvious to one of ordinary skill in the art.

### EXAMPLE 1

### Isolation of Phosphotyrosine-Containing Peptides from Extracts of Leukemia Cell Lines and Identification of Novel Phosphorylation Sites

In order to discover previously unknown Leukemia-related signal transduction protein phosphorylation sites, IAP isolation techniques were employed to identify phosphotyrosine- and/or phosphoserine-containing peptides in cell extracts from the following human Leukemia cell lines and patient cell lines: HT-93, KBM-3, SEM, KU-812, SUP-B15, BV-173, CMK, HEL, CLL-220, CLL-1202, CLL23LB4, MEC1, MEC2, M01043, K562, EOL1, HL60, CTV-1, REH, MV4-11, PL-21, and MKPL-1; or from the following cell lines expressing activated BCR-Abl wild-type and mutant kinases such as: Baf3-p210 BCR-Abl, Baf3-M351T-BCR-ABL, Baf3-E255K-BCR-Abl, Baf3-Y253F-BCR-Abl, Baf3-T3151-BCR-ABI, 3T3-v-Abl; or activated Flt3 kinase such as Baf3-FLT3.

Tryptic phosphotyrosine- and phosphoserine-containing peptides were purified and analyzed from extracts of each of the 29 cell lines mentioned above, as follows. Cells were cultured in DMEM medium or RPMI 1640 medium supplemented with 10% fetal bovine serum and penicillin/streptomycin. Cells were harvested by low speed centrifugation. After complete aspiration of medium, cells were resuspended in 1 mL lysis buffer per 1.25×10⁸ cells (20 mM HEPES pH 8.0, 9 M urea, 1 mM sodium vanadate, supplemented or not with 2.5 mM sodium pyro-phosphate, 1 mM β-glycerol-phosphate) and sonicated.

Sonicated cell lysates were cleared by centrifugation at 20,000×g, and proteins were reduced with DTT at a final concentration of 4.1 mM and alkylated with iodoacetamide at 8.3 mM. For digestion with trypsin, protein extracts were diluted in 20 mM HEPES pH 8.0 to a final concentration of 2 M urea and soluble TLCK-trypsin (Worthington) was added at 10-20 μg/mL. Digestion was performed for 1-2 days at room temperature.

Trifluoroacetic acid (TFA) was added to protein digests to a final concentration of 1%, precipitate was removed by centrifugation, and digests were loaded onto Sep-Pak C₁₈ columns (Waters) equilibrated with 0.1% TFA. A column volume of 0.7-1.0 ml was used per 2×10⁸ cells. Columns were washed with 15 volumes of 0.1% TFA, followed by 4 volumes of 5% acetonitrile (MeCN) in 0.1% TFA. Peptide fraction I was obtained by eluting columns with 2 volumes each of 8,12, and 15% MeCN in 0.1% TFA and combining the eluates. Fractions II and III were a combination of eluates after eluting columns with 18, 22, 25% MeCN in 0.1% TFA and with 30, 35, 40% MeCN in 0.1% TFA, respectively. All peptide fractions were lyophilized.

Peptides from each fraction corresponding to 2×10⁸ cells were dissolved in 1 ml of IAP buffer (20 mM Tris/HCl or 50 mM MOPS pH 7.2, mM sodium phosphate, 50 mM NaCl) and insoluble matter (mainly in peptide fractions III) was removed by centrifugation. IAP was performed on each peptide fraction separately. The phosphotyrosine monoclonal antibody P-Tyr-100 (Cell Signaling Technology, Inc., catalog number 9411) or the phospho-motif PxpSP rabbit monoclonal antibody (Cell Signaling Technology, Inc., catalog number 2325) (pS=phosphoserine) were coupled at 4 mg/ml beads to protein G or protein A agarose (Roche), respectively. Immobilized antibody (15 μl, 60 μg) was added as 1:1 slurry in IAP buffer to 1 ml of each peptide fraction, and the mixture was incubated overnight at 4° C. with gentle rotation. The immobilized antibody beads were washed three times with 1 ml IAP buffer and twice with 1 ml water, all at 4° C. Peptides were eluted from beads by incubation with 75 μl of 0.1% TFA at room temperature for 10 minutes.

Alternatively, one single peptide fraction was obtained from Sep-Pak C18 columns by elution with 2 volumes each of 10%, 15%, 20%, 25%, 30%, 35% and 40% acetonitirile in 0.1% TFA and combination of all eluates. IAP on this peptide fraction was performed as follows: After lyophilization, peptide was dissolved in 1.4 ml IAP buffer (MOPS pH 7.2, mM sodium phosphate, 50 mM NaCl) and insoluble matter was removed by centrifugation. Immobilized antibody (40 μl, 160 μg) was added as 1:1 slurry in IAP buffer, and the mixture was incubated overnight at 4° C. with gentle shaking. The immobilized antibody beads were washed three times with 1 ml IAP buffer and twice with 1 ml water, all at 4° C. Peptides were eluted from beads by incubation with 55 μl of 0.15% TFA at room temperature for 10 min (eluate 1), followed by a wash of the beads (eluate 2) with 45 μl of 0.15% TFA. Both eluates were combined.

### Analysis by LC-MS/MS Mass Spectrometry.

40 μl or more of IAP eluate were purified by 0.2 μl StageTips or ZipTips. Peptides were eluted from the microcolumns with 1 μl of 40% MeCN, 0.1% TFA (fractions I and II) or 1 μl of 60% MeCN, 0.1% TFA (fraction III) into 7.6 μl of 0.4% acetic acid/0.005% heptafluorobutyric acid. This sample was loaded onto a 10 cm×75 μm PicoFrit capillary column (New Objective) packed with Magic C18 AQ reversed-phase resin (Michrom Bioresources) using a Famos autosampler with an inert sample injection valve (Dionex). The column was then developed with a 45-min linear gradient of acetonitrile delivered at 200 nl/min (Ultimate, Dionex), and tandem mass spectra were collected in a data-dependent manner with an LCQ Deca XP Plus ion trap mass spectrometer essentially as described by Gygi et al., supra.

### Database Analysis & Assignments.

MS/MS spectra were evaluated using TurboSequest in the Sequest Browser package (v. 27, rev. 12) supplied as part of BioWorks 3.0 (ThermoFinnigan). Individual MS/MS spectra were extracted from the raw data file using the Sequest Browser program CreateDta, with the following settings: bottom MW, 700; top MW, 4,500; minimum number of ions, 20; minimum TIC, 4×10⁵; and precursor charge state, unspecified. Spectra were extracted from the beginning of the raw data file before sample injection to the end of the eluting gradient. The IonQuest and VuDta programs were not used to further select MS/MS spectra for Sequest analysis. MS/MS spectra were evaluated with the following TurboSequest parameters: peptide mass tolerance, 2.5; fragment ion tolerance, 0.0; maximum number of differential amino acids per modification, 4; mass type parent, average; mass type fragment, average; maximum number of internal cleavage sites, 10; neutral losses of water and ammonia from b and y ions were considered in the correlation analysis. Proteolytic enzyme was specified except for spectra collected from elastase digests.

Searches were performed against the NCBI human protein database (either as released on Apr. 29, 2003 and containing 37,490 protein sequences or as released on Feb. 23, 2004 and containing 27,175 protein sequences). Cysteine carboxamidomethylation was specified as a static modification, and phosphorylation was allowed as a variable modification on serine, threonine, and tyrosine residues or on tyrosine residues alone. It was determined that restricting phosphorylation to tyrosine residues had little effect on the number of phosphorylation sites assigned.

In proteomics research, it is desirable to validate protein identifications based solely on the observation of a single peptide in one experimental result, in order to indicate that the protein is, in fact, present in a sample. This has led to the development of statistical methods for validating peptide assignments, which are not yet universally accepted, and guidelines for the publication of protein and peptide identification results (see Carr et al., Mol. Cell. Proteomics 3: 531-533 (2004)), which were followed in this Example. However, because the immunoaffinity strategy separates phosphorylated peptides from unphosphorylated peptides, observing just one phosphopeptide from a protein is a common result, since many phosphorylated proteins have only one tyrosine-phosphorylated site. For this reason, it is appropriate to use additional criteria to validate phosphopeptide assignments. Assignments are likely to be correct if any of these additional criteria are met: (i) the same sequence is assigned to co-eluting ions with different charge states, since the MS/MS spectrum changes markedly with charge state; (ii) the site is found in more than one peptide sequence context due to sequence overlaps from incomplete proteolysis or use of proteases other than trypsin; (iii) the site is found in more than one peptide sequence context due to homologous but not identical protein isoforms; (iv) the site is found in more than one peptide sequence context due to homologous but not identical proteins among species; and (v) sites validated by MS/MS analysis of synthetic phosphopeptides corresponding to assigned sequences, since the ion trap mass spectrometer produces highly reproducible MS/MS spectra. The last criterion is routinely employed to confirm novel site assignments of particular interest.

All spectra and all sequence assignments made by Sequest were imported into a relational database. Assigned sequences were accepted or rejected following a conservative, two-step process. In the first step, a subset of high-scoring sequence assignments was selected by filtering for XCorr values of at least 1.5 for a charge state of +1, 2.2 for +2, and 3.3 for +3, allowing a maximum RSp value of 10. Assignments in this subset were rejected if any of the following criteria were satisfied: (i) the spectrum contained at least one major peak (at least 10% as intense as the most intense ion in the spectrum) that could not be mapped to the assigned sequence as an a, b, or y ion, as an ion arising from neutral-loss of water or ammonia from a b or y ion, or as a multiply protonated ion; (ii) the spectrum did not contain a series of b or y ions equivalent to at least six uninterrupted residues; or (iii) the sequence was not observed at least five times in all the studies we have conducted (except for overlapping sequences due to incomplete proteolysis or use of proteases other than trypsin). In the second step, assignments with below-threshold scores were accepted if the low-scoring spectrum showed a high degree of similarity to a high-scoring spectrum collected in another study, which simulates a true reference library-searching strategy. All spectra supporting the final list of 424 assigned sequences enumerated in Table 1/FIG. 2 herein were reviewed by at least three people to establish their credibility.

### EXAMPLE 2

### Production of Phospho-specific Polyclonal Antibodies for the Detection of Leukemia-related Signaling Protein Phosphorylation

Polyclonal antibodies that specifically bind a Leukemia-related signal transduction protein only when phosphorylated at the respective phosphorylation site disclosed herein (see Table 1/FIG. 2) are produced according to standard methods by first constructing a synthetic peptide antigen comprising the phosphorylation site sequence and then immunizing an animal to raise antibodies against the antigen, as further described below. Production of exemplary polyclonal antibodies is provided below.

A. FLT3 (tyrosine 955).

A 14 amino acid phospho-peptide antigen, ADAEEAMY*QNVDGR (where y*=phosphotyrosine) that corresponds to the sequence encompassing the tyrosine 955 phosphorylation site in human FLT3 kinase (see Row 371 of Table 1; SEQ ID NO: 370), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals to produce (and subsequently screen) phospho-specific FLT3(tyr955) polyclonal antibodies as described in Immunization/Screening below.

### B. CAMKK2 (Serine 331).

A 15 amino acid phospho-peptide antigen, ICPSLPYS*PVSSPQS (where s*=phosphoserine) that corresponds to the sequence encompassing the serine 331 phosphorylation site in human CAMKK2 kinase (see Row 331 of Table 1 (SEQ ID NO: 330)), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals to produce (and subsequently screen) phospho-specific CAMKK2(ser331) polyclonal antibodies as described in Immunization/Screening below.

### C. Crk (Tyrosine 251).

A 13 amino acid phospho-peptide antigen, RVPNAy*DKTALAL (where y*=phosphotyrosine) that corresponds to the sequence encompassing the tyrosine 251 phosphorylation site in human Crk protein (see Row 19 of Table 1 (SEQ ID NO: 18), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals to produce (and subsequently screen) phospho-specific Crk(tyr251) antibodies as described in Immunization/Screening below.

### Immunization/Screening.

A synthetic phospho-peptide antigen as described in A-C above is coupled to KLH, and rabbits are injected intradermally (ID) on the back with antigen in complete Freunds adjuvant (500 μg antigen per rabbit). The rabbits are boosted with same antigen in incomplete Freund adjuvant (250 μg antigen per rabbit) every three weeks. After the fifth boost, bleeds are collected. The sera are purified by Protein A-affinity chromatography by standard methods (see ANTIBODIES: A LABORATORY MANUAL, Cold Spring Harbor, supra.). The eluted immunoglobulins are further loaded onto a non-phosphorylated synthetic peptide antigen-resin Knotes column to pull out antibodies that bind the non-phosphorylated form of the phosphorylation site. The flow through fraction is collected and applied onto a phospho-synthetic peptide antigen-resin column to isolate antibodies that bind the phosphorylated form of the site. After washing the column extensively, the bound antibodies (i.e. antibodies that bind a phosphorylated peptide described in A-C above, but do not bind the non-phosphorylated form of the peptide) are eluted and kept in antibody storage buffer.

The isolated antibody is then tested for phospho-specificity using Western blot assay using an appropriate cell line that expresses (or overexpresses) target phospho-protein (i.e. phosphorylated FLT3, CAMKK2, or Crk), for example, SEM, M01043 and Baf3-E255K BCR-Abl cells, respectively. Cells are cultured in DMEM or RPMI supplemented with 10% FCS. Cell are collected, washed with PBS and directly lysed in cell lysis buffer. The protein concentration of cell lysates is then measured. The loading buffer is added into cell lysate and the mixture is boiled at 100° C. for 5 minutes. 20 μl (10 μg protein) of sample is then added onto 7.5% SDS-PAGE gel.

A standard Western blot may be performed according to the Immunoblotting Protocol set out in the CELL SIGNALING TECHNOLOGY, INC. 2003-04 Catalogue, p. 390. The isolated phospho-specific antibody is used at dilution 1:1000. Phosphorylation-site specificity of the antibody will be shown by binding of only the phosphorylated form of the target protein. Isolated phospho-specific polyclonal antibody does not (substantially) recognize the target protein when not phosphorylated at the appropriate phosphorylation site in the non-stimulated cells (e.g. FLT3 is not bound when not phosphorylated at tyrosine 955).

In order to confirm the specificity of the isolated antibody, different cell lysates containing various phosphorylated signal transduction proteins other than the target protein are prepared. The Western blot assay is performed again using these cell lysates. The phospho-specific polyclonal antibody isolated as described above is used (1:1000 dilution) to test reactivity with the different phosphorylated non-target proteins on Western blot membrane. The phospho-specific antibody does not significantly cross-react with other phosphorylated signal transduction proteins, although occasionally slight binding with a highly homologous phosphorylation-site on another protein may be observed. In such case the antibody may be further purified using affinity chromatography, or the specific immunoreactivity cloned by rabbit hybridoma technology.

### EXAMPLE 3

### Production of Phospho-Specific Monoclonal Antibodies for the Detection of Leukemia-Related Signaling Protein Phosphorylation

Monoclonal antibodies that specifically bind a Leukemia-related signal transduction protein only when phosphorylated at the respective phosphorylation site disclosed herein (see Table 1/FIG. 2) are produced according to standard methods by first constructing a synthetic peptide antigen comprising the phosphorylation site sequence and then immunizing an animal to raise antibodies against the antigen, and harvesting spleen cells from such animals to produce fusion hybridomas, as further described below. Production of exemplary monoclonal antibodies is provided below.

### A. ZAP70 (Tyrosine 397).

A 10 amino acid phospho-peptide antigen, HQLDNPy*IVR (where y*=phosphotyrosine) that corresponds to the sequence encompassing the tyrosine 397 phosphorylation site in human ZAP70 kinase (see Row 368 of Table 1 (SEQ ID NO: 367)), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals and harvest spleen cells for generation (and subsequent screening) of phospho-specific monoclonal ZAP70(tyr397) antibodies as described in Immunization/Fusion/Screening below.

### B. LRRK1 (Tyrosine 417).

A 10 amino acid phospho-peptide antigen, VTIy*SFTGNQ (where y*=phosphotyrosine) that corresponds to the sequence encompassing the tyrosine 417 phosphorylation site in human LRRK1 kinase (see Row 336 of Table 1 (SEQ ID NO: 335)), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals and harvest spleen cells for generation (and subsequent screening) of phospho-specific monoclonal LRRK1(tyr417) antibodies as described in Immunization/Fusion/Screening below.

### C. Elf-1 (Serine 187).

A 14 amino acid phospho-peptide antigen, KPPRPDs*PATTPNI (where s*=phosphoserine) that corresponds to the sequence encompassing the serine 187 phosphorylation site in human Elf-1 protein (see Row 272 of Table 1 (SEQ ID NO: 271)), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals and harvest spleen cells for generation (and subsequent screening) of phospho-specific monoclonal Elf-1(ser187) antibodies as described in Immunization/Fusion/Screening below.

### Immunization/Fusion/Screening.

A synthetic phospho-peptide antigen as described in A-C above is coupled to KLH, and BALB/C mice are injected intradermally (ID) on the back with antigen in complete Freunds adjuvant (e.g. 50 μg antigen per mouse). The mice are boosted with same antigen in incomplete Freund adjuvant (e.g. 25 μg antigen per mouse) every three weeks. After the fifth boost, the animals are sacrificed and spleens are harvested.

Harvested spleen cells are fused to SP2/0 mouse myeloma fusion partner cells according to the standard protocol of Kohler and Milstein (1975). Colonies originating from the fusion are screened by ELISA for reactivity to the phospho-peptide and non-phospho-peptide forms of the antigen and by Western blot analysis (as described in Example 1 above). Colonies found to be positive by ELISA to the phospho-peptide while negative to the non-phospho-peptide are further characterized by Western blot analysis. Colonies found to be positive by Western blot analysis are subcloned by limited dilution. Mouse ascites are produced from a single clone obtained from subcloning, and tested for phospho-specificity (against the ZAP70, LRRK1, or Elf-1 phospho-peptide antigen, as the case may be) on ELISA. Clones identified as positive on Western blot analysis using cell culture supernatant as having phospho-specificity, as indicated by a strong band in the induced lane and a weak band in the uninduced lane of the blot, are isolated and subcloned as clones producing monoclonal antibodies with the desired specificity.

Ascites fluid from isolated clones may be further tested by Western blot analysis. The ascites fluid should produce similar results on Western blot analysis as observed previously with the cell culture supernatant, indicating phospho-specificity against the phosphorylated target (e.g. Elf-1 phosphorylated at serine 187).

### EXAMPLE 4

### Production and Use of AQUA Peptides for the Quantification of Leukemia-related Signaling Protein Phosphorylation

Heavy-isotope labeled peptides (AQUA peptides (internal standards)) for the detection and quantification of a Leukemia-related signal transduction protein only when phosphorylated at the respective phosphorylation site disclosed herein (see Table 1/FIG. 2) are produced according to the standard AQUA methodology (see Gygi et al., Gerber et al., supra.) methods by first constructing a synthetic peptide standard corresponding to the phosphorylation site sequence and incorporating a heavy-isotope label. Subsequently, the MSⁿ and LC-SRM signature of the peptide standard is validated, and the AQUA peptide is used to quantify native peptide in a biological sample, such as a digested cell extract. Production and use of exemplary AQUA peptides is provided below.

### A. Tyk2 (Tyrosine 292).

An AQUA peptide comprising the sequence, LLAQAEGEPCy*IR (y*=phosphotyrosine; sequence incorporating ¹⁴C/¹⁵N-labeled leucine (indicated by bold L), which corresponds to the tyrosine 292 phosphorylation site in human Tyk2 kinase (see Row 367 in Table 1 (SEQ ID NO: 366)), is constructed according to standard synthesis techniques using, e.g., a Rainin/Protein Technologies, Inc., Symphony peptide synthesizer (see Merrifield, supra.) as further described below in Synthesis & MS/MS Signature. The Tyk2(tyr292) AQUA peptide is then spiked into a biological sample to quantify the amount of phosphorylated Tyk2(tyr292) in the sample, as further described below in Analysis & Quantification.

### B. GRK2 (Tyrosine 356).

An AQUA peptide comprising the sequence KKPHASVGTHGy*MAPEVLQK (y*=phosphotyrosine; sequence incorporating ¹⁴C/¹⁵N-labeled leucine (indicated by bold L), which corresponds to the tyrosine 356 phosphorylation site in human GRK2 kinase (see Row 335 in Table 1 (SEQ ID NO: 334)), is constructed according to standard synthesis techniques using, e.g., a Rainin/Protein Technologies, Inc., Symphony peptide synthesizer (see Merrifield, supra.) as further described below in Synthesis & MS/MS Signature. The GRK2(tyr356) AQUA peptide is then spiked into a biological sample to quantify the amount of phosphorylated GRK2(tyr356) in the sample, as further described below in Analysis & Quantification.

C. eIF4B (Tyrosine 211)

An AQUA peptide comprising the sequence, ARPATDSFDDy*PPR (y*=phosphotyrosine; sequence incorporating ¹⁴C/¹⁵N-labeled phenylalanine (indicated by bold F), which corresponds to the tyrosine 211 phosphorylation site in human eIF4B protein (see Row 397 in Table 1 (SEQ ID NO: 396)), is constructed according to standard synthesis techniques using, e.g., a Rainin/Protein Technologies, Inc., Symphony peptide synthesizer (see Merrifield, supra.) as further described below in Synthesis & MS/MS Signature. The eIF4B(tyr211) AQUA peptide is then spiked into a biological sample to quantify the amount of phosphorylated eIF4B(tyr211) in the sample, as further described below in Analysis & Quantification.

### D. NEDD4L (Serine 479).

An AQUA peptide comprising the sequence, DTLSNPQs*PQPSPYNSPKPQHK (s*=phosphoserine; sequence incorporating ¹⁴C/¹⁵N-labeled proline (indicated by bold P), which corresponds to the serine 479 phosphorylation site in human NEDD4L protein (see Row 164 in Table 1 (SEQ ID NO: 163)), is constructed according to standard synthesis techniques using, e.g., a Rainin/Protein Technologies, Inc., Symphony peptide synthesizer (see Merrifield, supra.) as further described below in Synthesis & MS/MS Signature. The NEDD4L(ser479) AQUA peptide is then spiked into a biological sample to quantify the amount of phosphorylated NEDD4L(ser479) in the sample, as further described below in Analysis & Quantification.

### Synthesis & MS/MS Spectra.

Fluorenylmethoxycarbonyl (Fmoc)-derivatized amino acid monomers may be obtained from AnaSpec (San Jose, Calif.). Fmoc-derivatized stable-isotope monomers containing one ¹⁵N and five to nine ¹³C atoms may be obtained from Cambridge Isotope Laboratories (Andover, Mass.). Preloaded Wang resins may be obtained from Applied Biosystems. Synthesis scales may vary from 5 to 25 μmol. Amino acids are activated in situ with 1-H-benzotriazolium, 1-bis(dimethylamino) methylene]-hexafluorophosphate (1-), 3-oxide:1-hydroxybenzotriazole hydrate and coupled at a 5-fold molar excess over peptide. Each coupling cycle is followed by capping with acetic anhydride to avoid accumulation of one-residue deletion peptide by-products. After synthesis peptide-resins are treated with a standard scavenger-containing trifluoroacetic acid (TFA)-water cleavage solution, and the peptides are precipitated by addition to cold ether. Peptides (i.e. a desired AQUA peptide described in A-D above) are purified by reversed-phase C18 HPLC using standard TFA/acetonitrile gradients and characterized by matrix-assisted laser desorption ionization-time of flight (Biflex III, Bruker Daltonics, Billerica, Mass.) and ion-trap (ThermoFinnigan, LCQ DecaXP) MS.

MS/MS spectra for each AQUA peptide should exhibit a strong y-type ion peak as the most intense fragment ion that is suitable for use in an SRM monitoring/analysis. Reverse-phase microcapillary columns (0.1 Å˜150-220 mm) are prepared according to standard methods. An Agilent 1100 liquid chromatograph may be used to develop and deliver a solvent gradient [0.4% acetic acid/0.005% heptafluorobutyric acid (HFBA)/7% methanol and 0.4% acetic acid/0.005% HFBA/65% methanol/35% acetonitrile] to the microcapillary column by means of a flow splitter. Samples are then directly loaded onto the microcapillary column by using a FAMOS inert capillary autosampler (LC Packings, San Francisco) after the flow split. Peptides are reconstituted in 6% acetic acid/0.01% TFA before injection.

### Analysis & Quantification.

Target protein (e.g. a phosphorylated protein of A-D above) in a biological sample is quantified using a validated AQUA peptide (as described above). The IAP method is then applied to the complex mixture of peptides derived from proteolytic cleavage of crude cell extracts to which the AQUA peptides have been spiked in.

LC-SRM of the entire sample is then carried out. MS/MS may be performed by using a ThermoFinnigan (San Jose, Calif.) mass spectrometer (LCQ DecaXP ion trap or TSQ Quantum triple quadrupole). On the DecaXP, parent ions are isolated at 1.6 m/z width, the ion injection time being limited to 150 ms per microscan, with two microscans per peptide averaged, and with an AGC setting of 1×10⁸; on the Quantum, Q1 is kept at 0.4 and Q3 at 0.8 m/z with a scan time of 200 ms per peptide. On both instruments, analyte and internal standard are analyzed in alternation within a previously known reverse-phase retention window; well-resolved pairs of internal standard and analyte are analyzed in separate retention segments to improve duty cycle. Data are processed by integrating the appropriate peaks in an extracted ion chromatogram (60.15 m/z from the fragment monitored) for the native and internal standard, followed by calculation of the ratio of peak areas multiplied by the absolute amount of internal standard (e.g., 500 fmol).

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. An isolated phosphorylation site-specific antibody that specifically binds a human Leukemia-related signaling protein selected from Column A of Table 1 only when phosphorylated at the tyrosine or serine listed in corresponding Column D of Table 1, comprised within the phosphorylatable peptide sequence listed in corresponding Column E of Table 1 (SEQ ID NOs: 1-424), wherein said antibody does not bind said signaling protein when not phosphorylated at said tyrosine or serine.

15. An isolated phosphorylation site-specific antibody that specifically binds a human Leukemia-related signaling protein selected from Column A of Table 1 only when not phosphorylated at the tyrosine or serine listed in corresponding Column D of Table 1, comprised within the phosphorylatable peptide sequence listed in corresponding Column E of Table 1 (SEQ ID NOs: 1-424), wherein said antibody does not bind said signaling protein when phosphorylated at said tyrosine or serine.

16. (canceled)

17. (canceled)

18. (canceled)

19. (canceled)

20. (canceled)

21. The antibody of claim 14, wherein said antibody specifically binds an Adaptor/Scaffold protein selected from Column A, Rows 2-78, of Table 1 only when phosphorylated at the tyrosine or serine listed in corresponding Column D, Rows 2-78, of Table 1, comprised within the phosphorylatable peptide sequence listed in corresponding Column E, Rows 2-78, of Table 1 (SEQ ID NOs: 1-77), wherein said antibody does not bind said protein when not phosphorylated at said tyrosine or serine.

22. (canceled)

23. The antibody of claim 14, wherein said antibody specifically binds a Cytoskeletal protein selected from Column A, Rows 98-150, of Table 1 only when phosphorylated at the tyrosine or serine listed in corresponding Column D, Rows 98-150, of Table 1, comprised within the phosphorylatable peptide sequence listed in corresponding Column E, Rows 98-150, of Table 1 (SEQ ID NOs: 97-149), wherein said antibody does not bind said protein when not phosphorylated at said tyrosine or serine.

24. (canceled)

25. The antibody of claim 14, wherein said antibody specifically binds a Cellular Metabolism Enzyme selected from Column A, Rows 152-177, of Table 1 only when phosphorylated at the tyrosine or serine listed in corresponding Column D, Rows 152-177, of Table 1, comprised within the phosphorylatable peptide sequence listed in corresponding Column E, Rows 152-177, of Table 1 (SEQ ID NOs: 151-176), wherein said antibody does not bind said protein when not phosphorylated at said tyrosine or serine.

26. (canceled)

27. The antibody of claim 14, wherein said antibody specifically binds a G Protein/GTP Activating/Guanine Nucleotide Exchange Factor protein selected from Column A, Rows 179-198, of Table 1 only when phosphorylated at the tyrosine or serine listed in corresponding Column D, Rows 179-198, of Table 1, comprised within the phosphorylatable peptide sequence listed in corresponding Column E, Rows 179-198, of Table 1 (SEQ ID NOs: 178-197), wherein said antibody does not bind said protein when not phosphorylated at said tyrosine or serine.

28. (canceled)

29. The antibody of claim 14, wherein said antibody specifically binds a Lipid Kinase selected from Column A, Rows 208-219, of Table 1 only when phosphorylated at the tyrosine or serine listed in corresponding Column D, Rows 208-219, of Table 1, comprised within the phosphorylatable peptide sequence listed in corresponding Column E, Rows 208-219 of Table 1 (SEQ ID NOs: 207-218), wherein said antibody does not bind said protein when not phosphorylated at said tyrosine or serine.

30. (canceled)

31. The antibody of claim 14, wherein said antibody specifically binds a Nuclear/DNA Repair/RNA Binding/Transcription protein selected from Column A, Rows 229-316, of Table 1 only when phosphorylated at the tyrosine or serine listed in corresponding Column D, Rows 229-316, of Table 1, comprised within the phosphorylatable peptide sequence listed in corresponding Column E, Rows 229-316, of Table 1 (SEQ ID NOs: 228-315), wherein said antibody does not bind said protein when not phosphorylated at said tyrosine or serine.

32. (canceled)

33. The antibody of claim 14, wherein said antibody specifically binds a Serine/Threonine Protein Kinase selected from Column A, Rows 327-345, of Table 1 only when phosphorylated at the tyrosine or serine listed in corresponding Column D, Rows 327-345, of Table 1, comprised within the phosphorylatable peptide sequence listed in corresponding Column E, Rows 327-345, of Table 1 (SEQ ID NOs: 326-344), wherein said antibody does not bind said protein when not phosphorylated at said tyrosine or serine.

34. (canceled)

35. The antibody of claim 14, wherein said antibody specifically binds a Tyrosine Protein Kinase selected from Column A, Rows 346-372, of Table 1 only when phosphorylated at the tyrosine listed in corresponding Column D, Rows 346-372, of Table 1, comprised within the phosphorylatable peptide sequence listed in corresponding Column E, Rows 346-372, of Table 1 (SEQ ID NOs: 345-371), wherein said antibody does not bind said protein when not phosphorylated at said tyrosine.

36. (canceled)

37. The antibody of claim 14, wherein said antibody specifically binds a Protein Phosphatase selected from Column A, Rows 373-378, of Table 1 only when phosphorylated at the tyrosine listed in corresponding Column D, Rows 373-378, of Table 1, comprised within the phosphorylatable peptide sequence listed in corresponding Column E, Rows 373-378, of Table 1 (SEQ ID NOs: 372-377), wherein said antibody does not bind said protein when not phosphorylated at said tyrosine.

38. (canceled)

39. The antibody of claim 14, wherein said antibody specifically binds a Translastion/Transporter protein selected from Column A, Rows 390-405, of Table 1 only when phosphorylated at the tyrosine or serine listed in corresponding Column D, Rows 390-405, of Table 1, comprised within the phosphorylatable peptide sequence listed in corresponding Column E, Rows 390-405, of Table 1 (SEQ ID NOs: 389-404), wherein said antibody does not bind said protein when not phosphorylated at said tyrosine or serine.

40. (canceled)

41. The antibody of claim 14, wherein said antibody specifically binds an Immunoglobulin Superfamily protein selected from Column A, Rows 199-203, of Table 1 only when phosphorylated at the tyrosine listed in corresponding Column D, Rows 199-203, of Table 1, comprised within the phosphorylatable peptide sequence listed in corresponding Column E, Rows 199-203, of Table 1 (SEQ ID NOs: 198-202), wherein said antibody does not bind said protein when not phosphorylated at said tyrosine.

42. (canceled)

43. The antibody of claim 14, wherein said antibody specifically binds an Inhibitor protein selected from Column A, Rows 204-207, of Table 1 only when phosphorylated at the tyrosine listed in corresponding Column D, Rows 204-207, of Table 1, comprised within the phosphorylatable peptide sequence listed in corresponding Column E, Rows 204-207, of Table 1 (SEQ ID NOs: 203-206), wherein said antibody does not bind said protein when not phosphorylated at said tyrosine.

44. (canceled)

45. (canceled)

46. (canceled)

47. (canceled)

48. (canceled)


SEQUENCE LISTING (424 sequences)

SEQ ID NO: 1 - protein, 25 aa, Homo sapiens
  MOD_RES (4)..(4): PHOSPHORYLATION; tyrosine at position 4 is phosphorylated
NTPYKTLEPVKPPTVPNDYMTSPAR

SEQ ID NO: 2 - protein, 25 aa, Homo sapiens
  MOD_RES (19)..(19)
  MOD_RES (19)..(19): PHOSPHORYLATION; tyrosine at position 19 is phosphorylated
NTPYKTLEPVKPPTVPNDYMTSPAR

SEQ ID NO: 3 - protein, 11 aa, Homo sapiens
  MOD_RES (6)..(6): PHOSPHORYLATION; tyrosine at position 6 is phosphorylated
ALIESYQNLTR

SEQ ID NO: 4 - protein, 20 aa, Homo sapiens
  MOD_RES (14)..(14): PHOSPHORYLATION; tyrosine at position 14 is phosphorylated
TLEPVRPPVVPNDYVPSPTR

SEQ ID NO: 5 - protein, 20 aa, Homo sapiens
  MOD_RES (12)..(12): PHOSPHORYLATION; serine at position 12 is phosphorylated
DALGDSLQVPVSPSSTTSSR

SEQ ID NO: 6 - protein, 24 aa, Homo sapiens
  MOD_RES (12)..(12): PHOSPHORYLATION; tyrosine at position 12 is phosphorylated
TGFTPLHIAAHYENLNVAQLLLNR

SEQ ID NO: 7 - protein, 16 aa, Homo sapiens
  MOD_RES (9)..(9): PHOSPHORYLATION; tyrosine at position 9 is phosphorylated
SQERPGNFYVSSESIR

SEQ ID NO: 8 - protein, 19 aa, Homo sapiens
  MOD_RES (14)..(14): PHOSPHORYLATION; tyrosine at position 14 is phosphorylated
HSQHLPAKVEFGVYESGPR

SEQ ID NO: 9 - protein, 25 aa, Homo sapiens
  MOD_RES (20)..(20): PHOSPHORYLATION; serine at position 20 is phosphorylated
VNHEPEPAGGATPGATLPKSPSQLR

SEQ ID NO: 10 - protein, 21 aa, Homo sapiens
  MOD_RES (3)..(3): PHOSPHORYLATION; tyrosine at position 3 is phosphorylated
NTYNQTALDIVNQFTTSQASR

SEQ ID NO: 11 - protein, 31 aa, Homo sapiens
  MOD_RES (15)..(15): PHOSPHORYLATION; tyrosine at position 15 is phosphorylated
YQVPNPQAAPRDTIYQVPPSYQNQGIYQVPT

SEQ ID NO: 12 - protein, 31 aa, Homo sapiens; identical to SEQ ID NO: 11
  MOD_RES (27)..(27): PHOSPHORYLATION; tyrosine at position 27 is phosphorylated

SEQ ID NO: 13 - protein, 23 aa, Homo sapiens
  MOD_RES (17)..(17): PHOSPHORYLATION; tyrosine at positioon 17 is phosphorylated
GPVFSVPVGEIKPQGVYDIPPTK

SEQ ID NO: 14 - protein, 28 aa, Homo sapiens
  MOD_RES (24)..(24): PHOSPHORYLATION; tysrosine at position 24 is phosphorylated
HQSLSPNHPPPQLGQSVGSQNDAYDVPR

SEQ ID NO: 15 - protein, 21 aa, Homo sapiens
  MOD_RES (10)..(10): PHOSPHORYLATION; tyrosine at position 10 is phosphorylated
ANPQERDGVYDVPLHNPPDAK

SEQ ID NO: 16 - protein, 23 aa, Homo sapiens
  MOD_RES (12)..(12): PHOSPHORYLATION; tyrosine at position 12 is phosphorylated
DLPPPPPPDRPYSVGAESRPQRK

SEQ ID NO: 17 - protein, 20 aa, Homo sapiens
  MOD_RES (11)..(11): PHOSPHORYLATION; tyrosine at position 11 is phosphorylated
DTCYSPKPSVYLSTPSSASK

SEQ ID NO: 18 - protein, 20 aa, Homo sapiens
  MOD_RES (6)..(6): PHOSPHORYLATION; tyrosine at position 6 is phosphorylated
RVPNAYDKTALALEVGELVK

SEQ ID NO: 19 - protein, 20 aa, Homo sapiens
  MOD_RES (15)..(15): PHOSPHORYLATION; tyrosine at position 15 is phosphorylated
VQLNVFDEQGEEDSYDLKGR

SEQ ID NO: 20 - protein, 12 aa, Homo sapiens
  MOD_RES (7)..(7): PHOSPHORYLATION; tyrosine at position 7 is phosphorylated
HPEIVGYSVPGR

SEQ ID NO: 21 - protein, 22 aa, Homo sapiens
  MOD_RES (12)..(12): PHOSPHORYLATION; tyrosine at position 12 is phosphorylated
QSRPCMEENELYSSAVTVGPHK

SEQ ID NO: 22 - protein, 18 aa, Homo sapiens
  MOD_RES (8)..(8): PHOSPHORYLATION; tyrosine at position 8 is phosphorylated
GWQPGTEYDNVVLKKGPK

SEQ ID NO: 23 - protein, 27 aa, Homo sapiens
  MOD_RES (12)..(12): PHOSPHORYLATION; tyrosine at position 12 is phosphorylated
RGLVPMEENSIYSSWQEVGEFPVVVQR

SEQ ID NO: 24 - protein, 34 aa, Homo sapiens
  MOD_RES (28)..(28): PHOSPHORYLATION; tyrosine at position 28 is phosphorylated
KMHLAEPGPQSLPLLLGPEPNDLASGLYASVCKR

SEQ ID NO: 25 - protein, 37 aa, Homo sapiens
  MOD_RES (11)..(11): PHOSPHORYLATION; tyrosine at position 11 is phosphorylated
ASGPPGNEHLYENLCVLEASPTLHGGEPEPHEGPGSR

SEQ ID NO: 26 - protein, 30 aa, Homo sapiens
  MOD_RES (8)..(8): PHOSPHORYLATION; tyrosine at position 8 is phosphorylated
SPTTSPIYHNGQDLSWPGPANDSTLEAQYR

SEQ ID NO: 27 - protein, 31 aa, Homo sapiens
  MOD_RES (29)..(29): PHOSPHORYLATION; tyrosine at position 29 is phosphorylated
SPTTSPIYHNGQDLSWPGPANDSTLEAQYRR

SEQ ID NO: 28 - protein, 20 aa, Homo sapiens
  MOD_RES (20)..(20): PHOSPHORYLATION; tyrosine at position 20 is phosphorylated
KIWDLADPEGKGFLDKQGFY

SEQ ID NO: 29 - protein, 14 aa, Homo sapiens
  MOD_RES (8)..(8): PHOSPHORYLATION; serine at position 8 is phosphorylated
SSLYFPRSPSANEK

SEQ ID NO: 30 - protein, 33 aa, Homo sapiens
  MOD_RES (19)..(19): PHOSPHORYLATION; tyrosine at position 19 is phosphorylated
KATEDFLGSSSGYSSEDDYVGYSDVDQQSSSSR

SEQ ID NO: 31 - protein, 33 aa, Homo sapiens; identical to SEQ ID NO: 30
  MOD_RES (22)..(22): PHOSPHORYLATION; tyrosine at position 22 is phosphorylated

SEQ ID NO: 32 - protein, 23 aa, Homo sapiens
  MOD_RES (20)..(20): PHOSPHORYLATION; tyrosine at position 20 is phosphorylated
NSSYVHGGLDSNGKPADAVYGQK

SEQ ID NO: 33 - protein, 34 aa, Homo sapiens
  MOD_RES (13)..(13): PHOSPHORYLATION; tyrosine at position 13 is phosphorylated
HGMNGFFQQQMIYDSPPSRAPSASVDSSLYNLPR

SEQ ID NO: 34 - protein, 20 aa, Homo sapiens
  MOD_RES (16)..(16): PHOSPHORYLATION; tyrosine at position 16 is phosphorylated
HVSISYDIPPTPGNTYQIPR

SEQ ID NO: 35 - protein, 27 aa, Homo sapiens
  MOD_RES (20)..(20): PHOSPHORYLATION; tyrosine at position 20 is phosphorylated
LAQGNGHCVNGISGQVHGFYSLPKPSR

SEQ ID NO: 36 - protein, 17 aa, Homo sapiens
  MOD_RES (14)..(14): PHOSPHORYLATION; tyrosine at position 14 is phosphorylated
GSLTGSETDNEDVYTFK

SEQ ID NO: 37 - protein, 24 aa, Homo sapiens
  MOD_RES (20)..(20): PHOSPHORYLATION; tyrosine at position 20 is phosphorylated
EFGDLLVDNMDVPATPLSAYQIPR

SEQ ID NO: 38 - protein, 18 aa, Homo sapiens
  MOD_RES (7)..(7): PHOSPHORYLATION; tyrosine at position 7 is phosphorylated
SAVGFDYKGEVEKHTSQK

SEQ ID NO: 39 - protein, 8 aa, Homo sapiens
  MOD_RES (3)..(3): PHOSPHORYLATION; tyrosine at position 3 is phosphorylated
LIYLVPEK

SEQ ID NO: 40 - protein, 12 aa, Homo sapiens
  MOD_RES (11)..(11): PHOSPHORYLATION; tyrosine at position 11 is phosphorylated
AVNKKPTSAAYS

SEQ ID NO: 41 - protein, 19 aa, Homo sapiens
  MOD_RES (10)..(10): PHOSPHORYLATION; tyrosine at position 10 is phosphorylated
VAYHPYPEDYGDIEIGSHR

SEQ ID NO: 42 - protein, 20 aa, Homo sapiens
  MOD_RES (7)..(7): PHOSPHORYLATION; tyrosine at position 7 is phosphorylated
HGSEEAYIDPIAMEYYNWGR

SEQ ID NO: 43 - protein, 20 aa, Homo sapiens
  MOD_RES (15)..(15): PHOSPHORYLATION; tyrosine at position 15 is phosphorylated
HGSEEAYIDPIAMEYYNWGR

SEQ ID NO: 44 - protein, 20 aa, Homo sapiens
  MOD_RES (16)..(16): PHOSPHORYLATION; tyrosine at position 16 is phosphorylated
HGSEEAYIDPIAMEYYNWGR

SEQ ID NO: 45 - protein, 17 aa, Homo sapiens
  MOD_RES (13)..(13): PHOSPHORYLATION; tyrosine at position 13 is phosphorylated
QENAQSSAAAQTYSLAR

SEQ ID NO: 46 - protein, 33 aa, Homo sapiens
  MOD_RES (24)..(24): PHOSPHORYLATION; tyrosine at position 24 is phosphorylated
RKPSVPDSASPADDSFVDPGERLYDLNMPAYVK

SEQ ID NO: 47 - protein, 32 aa, Homo sapiens
  MOD_RES (24)..(24): PHOSPHORYLATION; tyrosine at position 24 is phosphorylated
NYVTVMQNNPLTSGLEPSPPQCDYIRPSLTGK

SEQ ID NO: 48 - protein, 32 aa, Homo sapiens
  MOD_RES (23)..(23): PHOSPHORYLATION; tyrosine at position 23 is phosphorylated
DASPTPSTDAEYPANGSGADRIYDLNIPAFVK

SEQ ID NO: 49 - protein, 32 aa, Homo sapiens; identical to SEQ ID NO: 48
  MOD_RES (12)..(12): PHOSPHORYLATION; tyrosine at position 12 is phosphorylated

SEQ ID NO: 50 - protein, 24 aa, Homo sapiens
  MOD_RES (17)..(17): PHOSPHORYLATION; tyrosine at position 17 is phosphorylated
QHSLPSSEHLGADGGLYQIPPQPR

SEQ ID NO: 51 - protein, 18 aa, Homo sapiens
  MOD_RES (14)..(14): PHOSPHORYLATION; tyrosine at position 14 is phosphorylated
SVDGDQGLGMEGPYEVLK

SEQ ID NO: 52 - protein, 18 aa, Homo sapiens
  MOD_RES (14)..(14): PHOSPHORYLATION; tyrosine at position 14 is phosphorylated
DSSSQENMVEDCLYETVK

SEQ ID NO: 53 - protein, 26 aa, Homo sapiens
  MOD_RES (14)..(14): PHOSPHORYLATION; tyrosiine at position 14 is phosphorylated
NKSGQSLTVPESTYTSIQGDPQRSPS

SEQ ID NO: 54 - protein, 34 aa, Homo sapiens
  MOD_RES (30)..(30): PHOSPHORYLATION; tyrosine at position 30 is phosphorylated
SGQSLTVPESTYTSIQGDPQRSPSSCNDLYATVK

SEQ ID NO: 55 - protein, 28 aa, Homo sapiens
  MOD_RES (18)..(18): PHOSPHORYLATION; tyrosine at position 18 is phosphorylated
ATLGTNGHHGLVPKENDYESISDLQQGR

SEQ ID NO: 56 - protein, 11 aa, Homo sapiens
  MOD_RES (7)..(7): PHOSPHORYLATION; tyrosine at position 7 is phosphorylated
FSPDSQYIDNR

SEQ ID NO: 57 - protein, 19 aa, Homo sapiens
  MOD_RES (8)..(8): PHOSPHORYLATION; tyrosine at position 8 is phosphorylated
RIPDDPDYSVVEDYSLLYQ

SEQ ID NO: 58 - protein, 19 aa, Homo sapiens
  MOD_RES (18)..(18): PHOSPHORYLATION; tyrosine at position 18 is phosphorylated
RIPDDPDYSVVEDYSLLYQ

SEQ ID NO: 59 - protein, 36 aa, Homo sapiens
  MOD_RES (22)..(22): PHOSPHORYLATION; tyrosine at position 22 is phosphorylated
FILQDLGSDVIPEDDEERGELYDDVDHPAAVSSPQR

SEQ ID NO: 60 - protein, 16 aa, Homo sapiens
  MOD_RES (10)..(10): PHOSPHORYLATION; tyrosine at position 10 is phosphorylated
VHTDFTPSPYDTDSLK

SEQ ID NO: 61 - protein, 14 aa, Homo sapiens
  MOD_RES (7)..(7): PHOSPHORYLATION; tyrosine at position 7 is phosphorylated
VTIADDYSDPFDAK

SEQ ID NO: 62 - protein, 20 aa, Homo sapiens
  MOD_RES (16)..(16): PHOSPHORYLATION; serine at position 16 is phosphorylated
VHAAPAAPSATALPASPVAR

SEQ ID NO: 63 - protein, 26 aa, Homo sapiens
  MOD_RES (10)..(10): PHOSPHORYLATION; tyrosine at position 10 is phosphorylated
ASPSPSLSSYSDPDSGHYCQLQPPVR

SEQ ID NO: 64 - protein, 26 aa, Homo sapiens
  MOD_RES (18)..(18): PHOSPHORYLATION; tyrosine at position 18 is phosphorylated
ASPSPSLSSYSDPDSGHYCQLQPPVR

SEQ ID NO: 65 - protein, 12 aa, Homo sapiens
  MOD_RES (8)..(8): PHOSPHORYLATION; tyrosine at position 8 is phosphorylated
TTAVEIDYDSLK

SEQ ID NO: 66 - protein, 16 aa, Homo sapiens
  MOD_RES (10)..(10): PHOSPHORYLATION; tyrosine at position 10 is phosphorylated
VHTDFTPSPYDHDSLK

SEQ ID NO: 67 - protein, 23 aa, Homo sapiens
  MOD_RES (3)..(3): PHOSPHORYLATION; tyrosine at position 3 is phosphorylated
SAYEAGIQALKPPDAPGPDEAPK

SEQ ID NO: 68 - protein, 21 aa, Homo sapiens
  MOD_RES (4)..(4): PHOSPHORYLATION; tyrosine at position 4 is phosphorylated
EELYSKVTPRRNRQQRPGTIK

SEQ ID NO: 69 - protein, 19 aa, Homo sapiens
  MOD_RES (12)..(12): PHOSPHORYLATION; serine at position 12 is phosphorylated
ESMCSTPAFPVSPETPYVK

SEQ ID NO: 70 - protein, 13 aa, Homo sapiens
  MOD_RES (7)..(7): PHOSPHORYLATION; tyrosine at position 7 is phosphorylated
AGSLPNYATINGK

SEQ ID NO: 71 - protein, 22 aa, Homo sapiens
  MOD_RES (8)..(8): PHOSPHORYLATION; tyrosine at position 8 is phosphorylated
PKPSNPIYNEPDEPIAFYAMGR

SEQ ID NO: 72 - protein, 22 aa, Homo sapiens
  MOD_RES (18)..(18): PHOSPHORYLATION; tyrosine at position 18 is phosphorylated
PKPSNPIYNEPDEPIAFYAMGR

SEQ ID NO: 73 - protein, 35 aa, Homo sapiens
  MOD_RES (18)..(18): PHOSPHORYLATION; tyrosine at position 18 is phosphorylated
RYEPIQATPPPPPLPSQYAQPSQPVTSASLHIHSK

SEQ ID NO: 74 - protein, 13 aa, Homo sapiens
  MOD_RES (9)..(9): PHOSPHORYLATION; tyrosine at position 9 is phosphorylated
AEQLASVQYTLPK

SEQ ID NO: 75 - protein, 16 aa, Homo sapiens
  MOD_RES (11)..(11): PHOSPHORYLATION; tyrosine at position 11 is phosphorylated
IEIAQKHPDIYAVPIK

SEQ ID NO: 76 - protein, 15 aa, Homo sapiens
  MOD_RES (4)..(4): PHOSPHORYLATION; tyrosine at position 4 is phosphorylated
RQQYSDQDYHSSTEK

SEQ ID NO: 77 - protein, 15 aa, Homo sapiens
  MOD_RES (9)..(9): PHOSPHORYLATION; tyrosine at position 9 is phosphorylated
RQQYSDQDYHSSTEK

SEQ ID NO: 78 - protein, 19 aa, Homo sapiens
  MOD_RES (10)..(10): PHOSPHORYLATION; tyrosine at position 10 is phosphorylated
THYPAQQGEYQTHQPVYHK

SEQ ID NO: 79 - protein, 33 aa, Homo sapiens
  MOD_RES (5)..(5): PHOSPHORYLATION; serine at position 5 is phosphorylated
GTEPSPGGTPQPSRPVSPAGPPEGVPEEAQPPR

SEQ ID NO: 80 - protein, 13 aa, Homo sapiens
  MOD_RES (9)..(9): PHOSPHORYLATION; tyrosine at position 9 is phosphorylated
DFYFDENPYFENK

SEQ ID NO: 81 - protein, 22 aa, Homo sapiens
  MOD_RES (22)..(22): PHOSPHORYLATION; tyrosine at position 22 is phosphorylated
KYRGSIHDFPGFDPNQDAEALY

SEQ ID NO: 82 - protein, 25 aa, Homo sapiens
  MOD_RES (5)..(5): PHOSPHORYLATION; tyrosine at position 5 is phosphorylated
HAASYSSDSENQGSYSGVIPPPPGR

SEQ ID NO: 83 - protein, 29 aa, Homo sapiens
  MOD_RES (13)..(13): PHOSPHORYLATION; tyrosine at posiiton 13 is phosphorylated
ASYSSDSENQGSYSGVIPPPPGRGQVKKG

SEQ ID NO: 84 - protein, 22 aa, Homo sapiens
  MOD_RES (10)..(10): PHOSPHORYLATION; serine at position 10 is phosphorylated
AIPGDQHPESPVHTEPMGIQGR

SEQ ID NO: 85 - protein, 31 aa, Homo sapiens
  MOD_RES (8)..(8): PHOSPHORYLATION; tyrosine at position 8 is phosphorylated
TEDSIRDYEDGMEVDTTPTVAGQFEDADVDH

SEQ ID NO: 86 - protein, 18 aa, Homo sapiens
  MOD_RES (1)..(1): PHOSPHORYLATION; tyrosine at position 1 is phosphorylated
YNCCEEIYPDITYSLYIR

SEQ ID NO: 87 - protein, 18 aa, Homo sapiens
  MOD_RES (8)..(8): PHOSPHORYLATION; tyrosine at position 8 is phosphorylated
YNCCEEIYPDITYSLYIR

SEQ ID NO: 88 - protein, 11 aa, Homo sapiens
  MOD_RES (10)..(10): PHOSPHORYLATION; tyrosine at position 10 is phosphorylated
HFSGLEEAVYR

SEQ ID NO: 89 - protein, 14 aa, Homo sapiens
  MOD_RES (10)..(10): PHOSPHORYLATION; tyrosine at position 10 is phosphorylated
GILDTYLGKYWAIK

SEQ ID NO: 90 - protein, 19 aa, Homo sapiens
  MOD_RES (7)..(7): PHOSPHORYLATION; tyrosine at position 7 is phosphorylated
GEHSIVYLKPSYAFGSVGK

SEQ ID NO: 91 - protein, 13 aa, Homo sapiens
  MOD_RES (5)..(5): PHOSPHORYLATION; tyrosine at position 5 is phosphorylated
TTPSYVAFTDTER

SEQ ID NO: 92 - protein, 31 aa, Homo sapiens
  MOD_RES (14)..(14): PHOSPHORYLATION; tyrosine at position 14 is phosphorylated
ELEQVCNPIISGLYQGAGGPGPGGFGAQGPK

SEQ ID NO: 93 - protein, 21 aa, Homo sapiens
  MOD_RES (13)..(13): PHOSPHORYLATION; tyrosine at position 13 is phosphorylated
LVSSPCCIVTSTYGWTANMER

SEQ ID NO: 94 - protein, 20 aa, Homo sapiens
  MOD_RES (9)..(9): PHOSPHORYLATION; tyrosine at position 9 is phosphorylated
MDNKKRLAYAIIQFLHDQLR

SEQ ID NO: 95 - protein, 14 aa, Homo sapiens
  MOD_RES (4)..(4): PHOSPHORYLATION; tyrosine at position 4 is phosphorylated
VEKYTISQEAYDQR

SEQ ID NO: 96 - protein, 37 aa, Homo sapiens
  MOD_RES (31)..(31): PHOSPHORYLATION; tyrosine at position 31 is phosphorylated
YCPQGTVADGAPSGTGDCPDPGEVPEYPPYYQEEAGY

SEQ ID NO: 97 - protein, 11 aa, Homo sapiens
  MOD_RES (7)..(7): PHOSPHORYLATION; tyrosine at position 7 is phosphorylated
IWHHTFYNELR

SEQ ID NO: 98 - protein, 18 aa, Homo sapiens
  MOD_RES (6)..(6): PHOSPHORYLATION; tyrosine at position 6 is phosphorylated
WHHTFYNELRVAPEEHPV

SEQ ID NO: 99 - protein, 22 aa, Homo sapiens
  MOD_RES (4)..(4): PHOSPHORYLATION; tyrosine at position 4 is phosphorylated
KDLYANTVLSGGTTMYPGLADR

SEQ ID NO: 100 - protein, 17 aa, Homo sapiens
  MOD_RES (3)..(3): PHOSPHORYLATION; tyrosine at position 3 is phosphorylated
VTYVITIDGKPYSLHLR

SEQ ID NO: 101 - protein, 21 aa, Homo sapiens
  MOD_RES (11)..(11): PHOSPHORYLATION; tyrosine at position 11 is phosphorylated
IENPNQFVPLYTDPQEVLEMR

SEQ ID NO: 102 - protein, 11 aa, Homo sapiens
  MOD_RES (4)..(4): PHOSPHORYLATION; tyrosine at position 4 is phosphorylated
DITYFIQQLLR

SEQ ID NO: 103 - protein, 16 aa, Homo sapiens
  MOD_RES (5)..(5): PHOSPHORYLATION; tyrosine at position 5 is phosphorylated
DYNPYNYSDSISPFNK

SEQ ID NO: 104 - protein, 28 aa, Homo sapiens
  MOD_RES (23)..(23): PHOSPHORYLATION; tyrosine at position 23 is phosphorylated
NIILEEGKEILVGDVGQTVDDPYATFVK

SEQ ID NO: 105 - protein, 14 aa, Homo sapiens
  MOD_RES (4)..(4): PHOSPHORYLATION; tyrosine at position 4 is phosphorylated
YALYDATYETKESK

SEQ ID NO: 106 - protein, 14 aa, Homo sapiens
  MOD_RES (8)..(8): PHOSPHORYLATION; tyrosine at position 8 is phosphorylated
YALYDATYETKESK

SEQ ID NO: 107 - protein, 17 aa, Homo sapiens
  MOD_RES (15)..(15): PHOSPHORYLATION; tyrosine at position 15 is phosphorylated
NASTFEEVVQVPSAYQK

SEQ ID NO: 108 - protein, 15 aa, Homo sapiens
  MOD_RES (1)..(1): PHOSPHORYLATION; tyrosine at position 1 is phosphorylated
YYLCLQLRDDIVSGR

SEQ ID NO: 109 - protein, 15 aa, Homo sapiens
  MOD_RES (2)..(2): PHOSPHORYLATION; tyrosine at position 2 is phosphorylated
YYLCLQLRDDIVSGR

SEQ ID NO: 110 - protein, 23 aa, Homo sapiens
  MOD_RES (3)..(3): PHOSPHORYLATION; tyrosine at position 3 is phosphorylated
LIYGQDSAYQSIAHYRPISNVSR

SEQ ID NO: 111 - protein, 23 aa, Homo sapiens
  MOD_RES (9)..(9): PHOSPHORYLATION; tyrosine at position 9 is phosphorylated
LIYGQDSAYQSIAHYRPISNVSR

SEQ ID NO: 112 - protein, 29 aa, Homo sapiens
  MOD_RES (7)..(7): PHOSPHORYLATION; tyrosine at position 7 is phosphorylated
SSLGLSYYPTSSTSSVSSSSSSPSSWLTR

SEQ ID NO: 113 - protein, 20 aa, Homo sapiens
  MOD_RES (6)..(6): PHOSPHORYLATION; tyrosine at position 6 is phosphorylated
GDADMYDLPKKEDALLYQSK

SEQ ID NO: 114 - protein, 15 aa, Homo sapiens
  MOD_RES (6)..(6): PHOSPHORYLATION; tyrosine at position 6 is phosphorylated
GYNDDYYEESYFTTR

SEQ ID NO: 115 - protein, 17 aa, Homo sapiens
  MOD_RES (13)..(13): PHOSPHORYLATION; serine at position 13 is phosphorylated
SEVQQPVHPKPLSPDSR

SEQ ID NO: 116 - protein, 15 aa, Homo sapiens
  MOD_RES (5)..(5): PHOSPHORYLATION; serine at position 5 is phosphorylated
ETPHSPGVEDAPIAK

SEQ ID NO: 117 - protein, 10 aa, Homo sapiens
  MOD_RES (5)..(5): PHOSPHORYLATION; tyrosine at position 5 is phosphorylated
ANTAYHLHQR

SEQ ID NO: 118 - protein, 11 aa, Homo sapiens
  MOD_RES (9)..(9): PHOSPHORYLATION; tyrosine at position 9 is phosphorylated
TMSVSDFNYSR

SEQ ID NO: 119 - protein, 15 aa, Homo sapiens
  MOD_RES (12)..(12): PHOSPHORYLATION; tyrosine at position 12 is phosphorylated
SQEQLAAELAEYTAK

SEQ ID NO: 120 - protein, 16 aa, Homo sapiens
  MOD_RES (11)..(11): PHOSPHORYLATION; tyrosine at position 11 is phosphorylated
TAPPPPPPPVYEPVSY

SEQ ID NO: 121 - protein, 26 aa, Homo sapiens
  MOD_RES (15)..(15): PHOSPHORYLATION; tyrosine at position 15 is phosphorylated
LQVEPAVDTSGVQCYGPGIEGQGVFR

SEQ ID NO: 122 - protein, 39 aa, Homo sapiens
  MOD_RES (19)..(19): PHOSPHORYLATION; serine at position 19 is phosphorylated
TVSSPIPYTPSPSSSRPISPGLSYASHTVGFTPPTSLTR

SEQ ID NO: 123 - protein, 12 aa, Homo sapiens
  MOD_RES (9)..(9): PHOSPHORYLATION; serine at position 9 is phosphorylated
AGGPTTPLSPTR

SEQ ID NO: 124 - protein, 12 aa, Homo sapiens
  MOD_RES (9)..(9): PHOSPHORYLATION; serine at position 9 is phorphorylated
AGGPATPLSPTR

SEQ ID NO: 125 - protein, 22 aa, Homo sapiens
  MOD_RES (5)..(5): PHOSPHORYLATION; tyrosine at position 5 is phosphorylated
VQLVYATNIQEPNVYSEVQEPK

SEQ ID NO: 126 - protein, 22 aa, Homo sapiens
  MOD_RES (15)..(15): PHOSPHORYLATION; tyrosine at position 15 is phosphorylated
VQLVYATNIQEPNVYSEVQEPK

SEQ ID NO: 127 - protein, 13 aa, Homo sapiens
  MOD_RES (4)..(4): PHOSPHORYLATION; tyrosine at position 4 is phosphorylated
WANYHLENAGCNK

SEQ ID NO: 128 - protein, 19 aa, Homo sapiens
  MOD_RES (8)..(8): PHOSPHORYLATION; tyrosine at position 8 is phosphorylated
VDTDGNGYISFNELNDLFK

SEQ ID NO: 129 - protein, 14 aa, Homo sapiens
  MOD_RES (2)..(2): PHOSPHORYLATION; tyrosine at position 2 is phosphorylated
VYALPEDLVEVNPK

SEQ ID NO: 130 - protein, 23 aa, Homo sapiens
  MOD_RES (8)..(8): PHOSPHORYLATION; tyrosine at position 8 is phosphorylated
SAQPSPHYMAGPSSGQIYGPGPR

SEQ ID NO: 131 - protein, 26 aa, Homo sapiens
  MOD_RES (9)..(9): PHOSPHORYLATION; tyrosine at position 9 is phosphorylated
EGILNDDIYCPPETAVLLASYAVQSK

SEQ ID NO: 132 - protein, 13 aa, Homo sapiens
  MOD_RES (3)..(3): PHOSPHORYLATION; tyrosine at position 3 is phosphorylated
GQYHTLQAGFSSR

SEQ ID NO: 133 - protein, 21 aa, Homo sapiens
  MOD_RES (20)..(20): PHOSPHORYLATION; tyrosine at position 20 is phosphorylated
NNYALNTTATYAEPYRPIQYR

SEQ ID NO: 134 - protein, 18 aa, Homo sapiens
  MOD_RES (13)..(13): PHOSPHORYLATION; serine at position 13 is phosphorylated
SSSVGSSSSYPISPAVSR

SEQ ID NO: 135 - protein, 28 aa, Homo sapiens
  MOD_RES (13)..(13): PHOSPHORYLATION; tyrosine at position 13 is phosphorylated
LLEAAAQSTKGYYSPYSVSGSGSTAGSR

SEQ ID NO: 136 - protein, 32 aa, Homo sapiens
  MOD_RES (12)..(12): PHOSPHORYLATION; tyrosine at position 12 is phosphorylated
EIMPHIREKLCYITLDFEKEMATAASSSSLEK

SEQ ID NO: 137 - protein, 26 aa, Homo sapiens
  MOD_RES (20)..(20): PHOSPHORYLATION; tyrosine at position 20 is phosphorylated
AGTFQAFEQFGQQLLAHGHYASPEIK

SEQ ID NO: 138 - protein, 21 aa, Homo sapiens
  MOD_RES (9)..(9): PHOSPHORYLATION; tyrosine at position 9 is phosphorylated
ALSSEGKPYVTKEELYQNLTR

SEQ ID NO: 139 - protein, 22 aa, Homo sapiens
  MOD_RES (13)..(13): PHOSPHORYLATION; tyrosine at position 13 is phosphorylated
EVVAGSHELGQDYEHVTMLQER

SEQ ID NO: 140 - protein, 19 aa, Homo sapiens
  MOD_RES (12)..(12): PHOSPHORYLATION; tyrosine at position 12 is phosphorylated
IVSSSDVGHDEYSTQSLVK

SEQ ID NO: 141 - protein, 11 aa, Homo sapiens
  MOD_RES (3)..(3): PHOSPHORYLATION; tyrosine at position 3 is phosphorylated
FFYSDQNVDSR

SEQ ID NO: 142 - protein, 11 aa, Homo sapiens
  MOD_RES (9)..(9): PHOSPHORYLATION; tyrosine at position 9 is phosphorylated
KSTVLQQQYNR

SEQ ID NO: 143 - protein, 22 aa, Homo sapiens
  MOD_RES (9)..(9): PHOSPHORYLATION; tyrosine at position 9 is phosphorylated
FMVDNEAIYDICRRNLDIERPT

SEQ ID NO: 144 - protein, 14 aa, Homo sapiens
  MOD_RES (9)..(9): PHOSPHORYLATION; tyrosine at position 9 is phosphorylated
NLDIERPTYTNLNR

SEQ ID NO: 145 - protein, 33 aa, Homo sapiens
  MOD_RES (14)..(14): PHOSPHORYLATION; tyrosine at position 14 is phosphorylated
SEAREDMAALEKDYEEVGVDSVEGEGEEEGEEY

SEQ ID NO: 146 - protein, 14 aa, Homo sapiens
  MOD_RES (4)..(4): PHOSPHORYLATION; tyrosine at position 4 is phosphorylated
NSSYFVEWIPNNVK

SEQ ID NO: 147 - protein, 8 aa, Homo sapiens
  MOD_RES (2)..(2): PHOSPHORYLATION; tyrosine at position 2 is phosphorylated
SYVTTSTR

SEQ ID NO: 148 - protein, 8 aa, Homo sapiens
  MOD_RES (7)..(7): PHOSPHORYLATION; tyrosine at position 7 is phosphorylated
SFLDSGYR

SEQ ID NO: 149 - protein, 17 aa, Homo sapiens
  MOD_RES (5)..(5): PHOSPHORYLATION; tyrosine at position 5 is phosphorylated
VSSGYVPPPVATPFSSK

SEQ ID NO: 150 - protein, 19 aa, Homo sapiens
  MOD_RES (15)..(15): PHOSPHORYLATION; tyrosine at position 15 is phosphorylated
QMPIGGDVPALQLQYDHCK

SEQ ID NO: 151 - protein, 19 aa, Homo sapiens
  MOD_RES (16)..(16): PHOSPHORYLATION; tyrosine at position 16 is phosphorylated
AWGGKEENLKAAQEEYIKR

SEQ ID NO: 152 - protein, 20 aa, Homo sapiens
  MOD_RES (10)..(10): PHOSPHORYLATION; tyrosine at position 10 is phosphorylated
TDSDSDLQLYKEQGEGQGDR

SEQ ID NO: 153 - protein, 15 aa, Homo sapiens
  MOD_RES (8)..(8): PHOSPHORYLATION; tyrosine at position 8 is phosphorylated
GMYDGPVYEVPATPK

SEQ ID NO: 154 - protein, 21 aa, Homo sapiens
  MOD_RES (16)..(16): PHOSPHORYLATION; tyrosine at position 16 is phosphorylated
KIDPYINIDAGTFSPYEHGEV

SEQ ID NO: 155 - protein, 16 aa, Homo sapiens
  MOD_RES (6)..(6): PHOSPHORYLATION; serine at position 6 is phosphorylated
NSLPASPAHQLSSSPR

SEQ ID NO: 156 - protein, 22 aa, Homo sapiens
  MOD_RES (17)..(17): PHOSPHORYLATION; tyrosine at position 17 is phosphorylated
KPGMFFNPEESELDLTYGNRYK

SEQ ID NO: 157 - protein, 13 aa, Homo sapiens
  MOD_RES (6)..(6): PHOSPHORYLATION; tyrosine at position 6 is phosphorylated
EAMSAYNSHEEGR

SEQ ID NO: 158 - protein, 19 aa, Homo sapiens
  MOD_RES (8)..(8): PHOSPHORYLATION; tyrosine at position 8 is phosphorylated
WEQGQADYMGADSFDNIKR

SEQ ID NO: 159 - protein, 21 aa, Homo sapiens
  MOD_RES (11)..(11): PHOSPHORYLATION; tyrosine at position 11 is phosphorylated
IANDNSLNHEYLPILGLAEFR

SEQ ID NO: 160 - protein, 17 aa, Homo sapiens
  MOD_RES (13)..(13): PHOSPHORYLATION; tyrosine at position 13 is phosphorylated
LLIVSNPVDILTYVAWK

SEQ ID NO: 161 - protein, 17 aa, Homo sapiens
  MOD_RES (5)..(5): PHOSPHORYLATION; tyrosine at position 5 is phosphorylated
DQLIYNLLKEEQTPQNK

SEQ ID NO: 162 - protein, 15 aa, Homo sapiens
  MOD_RES (10)..(10): PHOSPHORYLATION; serine at position 10 is phosphorylated
VLTANSNPSSPSAAK

SEQ ID NO: 163 - protein, 22 aa, Homo sapiens
  MOD_RES (8)..(8): PHOSPHORYLATION; serine at position 8 is phosphorylated
DTLSNPQSPQPSPYNSPKPQHK

SEQ ID NO: 164 - protein, 22 aa, Homo sapiens
  MOD_RES (12)..(12): PHOSPHORYLATION; serine at position 12 is phosphorylated
DTLSNPQSPQPSPYNSPKPQHK

SEQ ID NO: 165 - protein, 22 aa, Homo sapiens
  MOD_RES (16)..(16): PHOSPHORYLATIONserine at position 16 is phosphorylated
DTLSNPQSPQPSPYNSPKPQHK

SEQ ID NO: 166 - protein, 14 aa, Homo sapiens
  MOD_RES (1)..(1): PHOSPHORYLATION; tyrosine at position 1 is phosphorylated
YHGHSMSDPGVSYR

SEQ ID NO: 167 - protein, 14 aa, Homo sapiens
  MOD_RES (13)..(13): PHOSPHORYLATION; tyrosine at position 13 is phosphorylated
YHGHSMSDPGVSYR

SEQ ID NO: 168 - protein, 11 aa, Homo sapiens
  MOD_RES (4)..(4): PHOSPHORYLATION; tyrosine at position 4 is phosphorylated
IALYETPTGWK

SEQ ID NO: 169 - protein, 10 aa, Homo sapiens
  MOD_RES (2)..(2): PHOSPHORYLATION; tyrosine at position 2 is phosphorylated
HYGGLTGLNK

SEQ ID NO: 170 - protein, 15 aa, Homo sapiens
  MOD_RES (11)..(11): PHOSPHORYLATION; tyrosine at position 11 is phosphorylated
TGFSTSPESPYTHWK

SEQ ID NO: 171 - protein, 21 aa, Homo sapiens
  MOD_RES (4)..(4): PHOSPHORYLATION; tyrosine at position 4 is phosphorylated
EKTYSECEDGTYSPEISWHHR

SEQ ID NO: 172 - protein, 19 aa, Homo sapiens
  MOD_RES (10)..(10): PHOSPHORYLATION; tyrosine at position 10 is phosphorylated
TYSECEDGTYSPEISWHHR

SEQ ID NO: 173 - protein, 21 aa, Homo sapiens
  MOD_RES (7)..(7): PHOSPHORYLATION; tyrosine at position 7 is phosphorylated
ITLDNAYMEKCDENILWLDYK

SEQ ID NO: 174 - protein, 34 aa, Homo sapiens
  MOD_RES (28)..(28): PHOSPHORYLATION; tyrosine at position 28 is phosphorylated
AEGSDVANAVLDGADCIMLSGETAKGDYPLEAVR

SEQ ID NO: 175 - protein, 10 aa, Homo sapiens
  MOD_RES (7)..(7): PHOSPHORYLATION; tyrosine at position 7 is phosphorylated
SKFDNLYGCR

SEQ ID NO: 176 - protein, 11 aa, Homo sapiens
  MOD_RES (10)..(10): PHOSPHORYLATION; tyrosine at position 10 is phosphorylated
LQELGGTLEYR

SEQ ID NO: 177 - protein, 21 aa, Homo sapiens
  MOD_RES (15)..(15): PHOSPHORYLATION; tyrosine at position 15 is phosphorylated
VLCTRPSGNSAEEYYENVPCK

SEQ ID NO: 178 - protein, 24 aa, Homo sapiens
  MOD_RES (8)..(8): PHOSPHORYLATION; tyrosine at position 8 is phosphorylated
GKVSYQDYEIEISDASEVEKEINK

SEQ ID NO: 179 - protein, 13 aa, Homo sapiens
  MOD_RES (8)..(8): PHOSPHORYLATION; tyrosine at position 8 is phosphorylated
LHMDRNPYYGGES

SEQ ID NO: 180 - protein, 18 aa, Homo sapiens
  MOD_RES (13)..(13): PHOSPHORYLATION; tyrosine at position 13 is phosphorylated
LYRTDDYLDQPCYETINR

SEQ ID NO: 181 - protein, 16 aa, Homo sapiens
  MOD_RES (14)..(14): PHOSPHORYLATION; tyrosine at position 14 is phosphorylated
SSSFSSWDDGADSYWK

SEQ ID NO: 182 - protein, 34 aa, Homo sapiens
  MOD_RES (25)..(25): PHOSPHORYLATION; tyrosine at position 25 is phosphorylated
HSYPLSSTSGNADSSAVSSQAISPYACFYGASAK

SEQ ID NO: 183 - protein, 12 aa, Homo sapiens
  MOD_RES (7)..(7): PHOSPHORYLATION; tyrosine at position 7 is phosphorylated
MQDIPIYANVHK

SEQ ID NO: 184 - protein, 17 aa, Homo sapiens
  MOD_RES (2)..(2): PHOSPHORYLATION; tyrosine at position 2 is phosphorylated
HYSVVLPTVSHSGFLYK

SEQ ID NO: 185 - protein, 17 aa, Homo sapiens
  MOD_RES (16)..(16): PHOSPHORYLATION; tyrosine at position 16 is phosphorylated
HYSVVLPTVSHSGFLYK

SEQ ID NO: 186 - protein, 25 aa, Homo sapiens
  MOD_RES (24)..(24): PHOSPHORYLATION; tyrosine at position 24 is phosphorylated
AAASMGDTLSEQQLGDSDIPVIVYR

SEQ ID NO: 187 - protein, 25 aa, Homo sapiens
  MOD_RES (9)..(9): PHOSPHORYLATION; tyrosine at position 9 is phosphorylated
QNSTPESDYDNTACDPEPDDTGSTR

SEQ ID NO: 188 - protein, 25 aa, Homo sapiens
  MOD_RES (7)..(7): PHOSPHORYLATION; tyrosine at position 7 is phosphorylated
SPDVGLYGVIPECGETYHSDLAEAK

SEQ ID NO: 189 - protein, 14 aa, Homo sapiens
  MOD_RES (8)..(8): PHOSPHORYLATION; serine at position 8 is phosphorylated
ATHPPPASPSSLVK

SEQ ID NO: 190 - protein, 19 aa, Homo sapiens
  MOD_RES (14)..(14): PHOSPHORYLATION; tyrosine at position 14 is phosphorylated
ALPAQVDDPPEPVYANIER

SEQ ID NO: 191 - protein, 12 aa, Homo sapiens
  MOD_RES (10)..(10): PHOSPHORYLATION; serine at position 10 is phosphorylated
FLMPEAYPSSPR

SEQ ID NO: 192 - protein, 14 aa, Homo sapiens
  MOD_RES (8)..(8): PHOSPHORYLATION; tyrosine at position 8 is phosphorylated
ERPSSAIYPSDSFR

SEQ ID NO: 193 - protein, 19 aa, Homo sapiens
  MOD_RES (8)..(8): PHOSPHORYLATION; serine at position 8 is phosphorylated
QNTASPGSPVNSHLPGSPK

SEQ ID NO: 194 - protein, 19 aa, Homo sapiens
  MOD_RES (12)..(12): PHOSPHORYLATION; serine at position 12 is phosphorylated
QNTASPGSPVNSHLPGSPK

SEQ ID NO: 195 - protein, 29 aa, Homo sapiens
  MOD_RES (24)..(24): PHOSPHORYLATION; tyrsosine at position 24 is phosphorylated
EGVFSMGNNSHGQCGRKVVEDEVYSESHK

SEQ ID NO: 196 - protein, 21 aa, Homo sapiens
  MOD_RES (11)..(11): PHOSPHORYLATION; tyrosine at position 11 is phosphorylated
GNLYSFGCPEYGQLGHNSDGK

SEQ ID NO: 197 - protein, 17 aa, Homo sapiens
  MOD_RES (16)..(16): PHOSPHORYLATION; tyrosine at position 16 is phosphorylated
VGWFPANYVEEDYSEYC

SEQ ID NO: 198 - protein, 28 aa, Homo sapiens
  MOD_RES (13)..(13): PHOSPHORYLATION; tyrosine at position 13 is phosphorylated
VTPPPGSGPQNQYGNVLSLPTPTSGLGR

SEQ ID NO: 199 - protein, 28 aa, Homo sapiens
  MOD_RES (22)..(22): PHOSPHORYLATION; tyrosine at position 22 is phosphorylated
KRQVGDYENVIPDFPEDEGIHYSELIQF

SEQ ID NO: 200 - protein, 17 aa, Homo sapiens
  MOD_RES (10)..(10): PHOSPHORYLATION; tyrosine at position 10 is phosphorylated
NAQPTESRIYDEIPQSK

SEQ ID NO: 201 - protein, 11 aa, Homo sapiens
  MOD_RES (5)..(5): PHOSPHORYLATION; tyrosine at position 5 is phosphorylated
SDGVYTGLSTR

SEQ ID NO: 202 - protein, 19 aa, Homo sapiens
  MOD_RES (12)..(12): PHOSPHORYLATION; tyrosine at position 12 is phosphorylated
TILKEDPANTVYSTVEIPK

SEQ ID NO: 203 - protein, 19 aa, Homo sapiens
  MOD_RES (11)..(11): PHOSPHORYLATION; tyrosine at position 11 is phosphorylated
KGPDEAEESQYDSGIESLR

SEQ ID NO: 204 - protein, 19 aa, Homo sapiens
  MOD_RES (10)..(10): PHOSPHORYLATION; tyrosine at position  10 is phosphorylated
ILGDMQPGDYFDLVLFGTR

SEQ ID NO: 205 - protein, 31 aa, Homo sapiens
  MOD_RES (12)..(12): PHOSPHORYLATION; tyrosine at position 12 is phosphorylated
EEIQDEEDDDDYVEEGEEEEEEEEGGLRGEK

SEQ ID NO: 206 - protein, 19 aa, Homo sapiens
  MOD_RES (4)..(4): PHOSPHORYLATION; tyrosine at position 4 is phosphorylated
ELTYQNTDLSEIKEEEQVK

SEQ ID NO: 207 - protein, 11 aa, Homo sapiens
  MOD_RES (4)..(4): PHOSPHORYLATION; tyrosine at position 4 is phosphorylated
KQPYYYPPFDK

SEQ ID NO: 208 - protein, 11 aa, Homo sapiens
  MOD_RES (5)..(5): PHOSPHORYLATION; tyrosine at position 5 is phosphorylated
KQPYYYPPFDK

SEQ ID NO: 209 - protein, 11 aa, Homo sapiens
  MOD_RES (6)..(6): PHOSPHORYLATION; tyrosine at position 6 is phosphorylated
KQPYYYPPFDK

SEQ ID NO: 210 - protein, 23 aa, Homo sapiens
  MOD_RES (20)..(20): PHOSPHORYLATION; tyrosine at position 20 is phosphorylated
EALSDLQSPLNPCVILSELYVEK

SEQ ID NO: 211 - protein, 30 aa, Homo sapiens
  MOD_RES (24)..(24): PHOSPHORYLATION; tyrosine at position 24 is phosphorylated
SNPNTDSAAALLICLPEVAPHPVYYPALEK

SEQ ID NO: 212 - protein, 30 aa, Homo sapiens; identical to SEQ ID NO: 211
  MOD_RES (25)..(25): PHOSPHORYLATION; tyrsosine at position  25 is phosphorylated

SEQ ID NO: 213 - protein, 15 aa, Homo sapiens
  MOD_RES (6)..(6): PHOSPHORYLATION; tyrosine at position 6 is phosphorylated
GSGELYEHEKDLVWK

SEQ ID NO: 214 - protein, 19 aa, Homo sapiens
  MOD_RES (9)..(9): PHOSPHORYLATION; tyrosine at position 9 is phosphorylated
ERVPFILTYDFVHVIQQGK

SEQ ID NO: 215 - protein, 11 aa, Homo sapiens
  MOD_RES (4)..(4): PHOSPHORYLATION; tyrosine at position 4 is phosphorylated
LHEYNTQFQEK

SEQ ID NO: 216 - protein, 13 aa, Homo sapiens
  MOD_RES (8)..(8): PHOSPHORYLATION; tyrosine at position 8 is phosphorylated
SREYDRLYEEYTR

SEQ ID NO: 217 - protein, 9 aa, Homo sapiens
  MOD_RES (6)..(6): PHOSPHORYLATION; tyrosine at position 6 is phosphorylated
VYHQQYQDK

SEQ ID NO: 218 - protein, 14 aa, Homo sapiens
  MOD_RES (7)..(7): PHOSPHORYLATION; tyrosine at position 7 is phosphorylated
GADSAYYQVGQTGK

SEQ ID NO: 219 - protein, 17 aa, Homo sapiens
  MOD_RES (12)..(12): PHOSPHORYLATION; tyrosine at position 12 is phosphorylated
GWQYSDHMENVYGYLMK

SEQ ID NO: 220 - protein, 15 aa, Homo sapiens
  MOD_RES (7)..(7): PHOSPHORYLATION; tyrosine at position 7 is phosphorylated
SGDSEVYQLGDVSQK

SEQ ID NO: 221 - protein, 28 aa, Homo sapiens
  MOD_RES (10)..(10): PHOSPHORYLATION; serine at position 10 is phosphorylated
SKPIPIMPASPQKGHAVNLLDVPVPVAR

SEQ ID NO: 222 - protein, 18 aa, Homo sapiens
  MOD_RES (10)..(10): PHOSPHORYLATION; tyrosine at position 10 is phosphorylated
KRHEMPPHIYAITDTAYR

SEQ ID NO: 223 - protein, 15 aa, Homo sapiens
  MOD_RES (9)..(9): PHOSPHORYLATION; tyrosine at position 9 is phosphorylated
ALELDSNLYRIGQSK

SEQ ID NO: 224 - protein, 15 aa, Homo sapiens
  MOD_RES (7)..(7): PHOSPHORYLATION; tyrosine at position 7 is phosphorylated
NKDQGTYEDYVEGLR

SEQ ID NO: 225 - protein, 15 aa, Homo sapiens
  MOD_RES (10)..(10): PHOSPHORYLATION; tyrosine at position 10 is phosphorylated
NKDQGTYEDYVEGLR

SEQ ID NO: 226 - protein, 29 aa, Homo sapiens
  MOD_RES (6)..(6): PHOSPHORYLATION; tyrosine at position 6 is phosphorylated
GPQPNYESPYPGAPTFGSQPGPPQPLPPK

SEQ ID NO: 227 - protein, 29 aa, Homo sapiens
  MOD_RES (10)..(10): PHOSPHORYLATION; tyrosine at position 10 is phosphoryalted
GPQPNYESPYPGAPTFGSQPGPPQPLPPK

SEQ ID NO: 228 - protein, 10 aa, Homo sapiens
  MOD_RES (5)..(5): PHOSPHORYLATION; tyrosine at position 5 is phosphorylated
STCIYGGAPK

SEQ ID NO: 229 - protein, 21 aa, Homo sapiens
  MOD_RES (8)..(8): PHOSPHORYLATION; tyrosine at position 8 is phosphorylated
APKEEADYEDDFLEYDQEHIR

SEQ ID NO: 230 - protein, 21 aa, Homo sapiens
  MOD_RES (15)..(15): PHOSPHORYLATION; tyrosine at position 15 is phosphorylated
APKEEADYEDDFLEYDQEHIR

SEQ ID NO: 231 - protein, 18 aa, Homo sapiens
  MOD_RES (13)..(13): PHOSPHORYLATION; tyrosine at position 13 is phosphorylated
HINLPLPAPHAQYAIPNR

SEQ ID NO: 232 - protein, 16 aa, Homo sapiens
  MOD_RES (10)..(10): PHOSPHORYLATION; serine at position 10 is phosphorylated
NSCNVLHPQSPNNSNR

SEQ ID NO: 233 - protein, 29 aa, Homo sapiens
  MOD_RES (11)..(11): PHOSPHORYLATION; serine at position 11 is phoshorylated
AAAAGLGHPASPGGSEDGPPGSEEEDAAR

SEQ ID NO: 234 - protein, 29 aa, Homo sapiens
  MOD_RES (23)..(23): PHOSPHORYLATION; tyrosine at position 23 is phosphorylated
QVEEQSAAANEEVLFPFCREPSYFEIPTK

SEQ ID NO: 235 - protein, 18 aa, Homo sapiens
  MOD_RES (15)..(15): PHOSPHORYLATION; tyrosine at position 15 is phosphorylated
NNNDILPNSQEELLYSSK

SEQ ID NO: 236 - protein, 17 aa, Homo sapiens
  MOD_RES (7)..(7): PHOSPHORYLATION; tyrosine at position 7 is phosphorylated
LQKGQKYFDSGDYNMAK

SEQ ID NO: 237 - protein, 14 aa, Homo sapiens
  MOD_RES (10)..(10): PHOSPHORYLATION; tyrosine at position 10 is phosphorylated
SGGYGGSRDYYSSR

SEQ ID NO: 238 - protein, 14 aa, Homo sapiens
  MOD_RES (11)..(11): PHOSPHORYLATION; tyrosine at position 11 is phosphorylated
SGGYGGSRDYYSSR

SEQ ID NO: 239 - protein, 18 aa, Homo sapiens
  MOD_RES (6)..(6): PHOSPHORYLATION; tyrosine at position 6 is phosphorylated
SSGGSYRDSYDSYATHNE

SEQ ID NO: 240 - protein, 18 aa, Homo sapiens
  MOD_RES (10)..(10): PHOSPHORYLATION; tyrosine at position 10 is phosphorylated
SSGGSYRDSYDSYATHNE

SEQ ID NO: 241 - protein, 18 aa, Homo sapiens
  MOD_RES (13)..(13): PHOSPHORYLATION; tyrosine at position 13 is phosphorylated
SSGGSYRDSYDSYATHNE

SEQ ID NO: 242 - protein, 19 aa, Homo sapiens
  MOD_RES (6)..(6): PHOSPHORYLATION; tyrosine at position 6 is phosphorylated
TGAPQYGSYGTAPVNLNIK

SEQ ID NO: 243 - protein, 19 aa, Homo sapiens
  MOD_RES (9)..(9): PHOSPHORYLATION; tyrosine at position 9 is phosphorylated
TGAPQYGSYGTAPVNLNIK

SEQ ID NO: 244 - protein, 28 aa, Homo sapiens
  MOD_RES (21)..(21): PHOSPHORYLATION; serine at position 21 is phosphorylated
GFGDGYNGYGGGPGGGNFGGSPGYGGGR

SEQ ID NO: 245 - protein, 25 aa, Homo sapiens
  MOD_RES (22)..(22): PHOSPHORYLATION; tyrosine at position 22 is phosphorylated
NMGGPYGGGNYGPGGSGGSGGYGGR

SEQ ID NO: 246 - protein, 22 aa, Homo sapiens
  MOD_RES (19)..(19): PHOSPHORYLATION; tyrosine at position 19 is phosphorylated
SSGSPYGGGYGSGGGSGGYGSR

SEQ ID NO: 247 - protein, 16 aa, Homo sapiens
  MOD_RES (6)..(6): PHOSPHORYLATION; serine at position 6 is phosphorylated
HTGPNSPDTANDGFVR

SEQ ID NO: 248 - protein, 14 aa, Homo sapiens
  MOD_RES (4)..(4): PHOSPHORYLATION; tyrosine at position 4 is phosphorylated
STAYEDYYYHPPPR

SEQ ID NO: 249 - protein, 14 aa, Homo sapiens
  MOD_RES (8)..(8): PHOSPHORYLATION; tyrosine at position 8 is phosphorylated
STAYEDYYYHPPPR

SEQ ID NO: 250 - protein, 14 aa, Homo sapiens
  MOD_RES (9)..(9): PHOSPHORYLATION; tyrosine at position 9 is phosphorylated
STAYEDYYYHPPPR

SEQ ID NO: 251 - protein, 18 aa, Homo sapiens
  MOD_RES (14)..(14): PHOSPHORYLATION; tyrosine at position 14 is phosphorylated
NQGGYGGSSSSSSYGSGR

SEQ ID NO: 252 - protein, 12 aa, Homo sapiens
  MOD_RES (5)..(5): PHOSPHORYLATION; tyrosine at position 5 is phosphorylated
GQPIYIQFSNHK

SEQ ID NO: 253 - protein, 16 aa, Homo sapiens
  MOD_RES (13)..(13): PHOSPHORYLATION; tyrosine at position 13 is phosphorylated
HNPTVTGQQEQTYLPK

SEQ ID NO: 254 - protein, 16 aa, Homo sapiens
  MOD_RES (3)..(3): PHOSPHORYLATION; tyrosine at position 3 is phosphorylated
ALYDTFSAFGNILSCK

SEQ ID NO: 255 - protein, 10 aa, Homo sapiens
  MOD_RES (6)..(6): PHOSPHORYLATION; tyrosine at position 6 is phosphorylated
SSFSHYSGLK

SEQ ID NO: 256 - protein, 16 aa, Homo sapiens
  MOD_RES (6)..(6): PHOSPHORYLATION; tyrosine at position 6 is phosphorylated
TIQGQYAIPQPDLTKL

SEQ ID NO: 257 - protein, 15 aa, Homo sapiens
  MOD_RES (9)..(9): PHOSPHORYLATION; tyrosine at position 9 is phosphorylated
YYDSRPGGYGYGYGR

SEQ ID NO: 258 - protein, 15 aa, Homo sapiens
  MOD_RES (8)..(8): PHOSPHORYLATION; serine at position 8 is phosphorylated
VKVDGPRSPSYGRSR

SEQ ID NO: 259 - protein, 15 aa, Homo sapiens
  MOD_RES (14)..(14): PHOSPHORYLATION; serine at position 14 is phosphorylated
VKVDGPRSPSYGRSR

SEQ ID NO: 260 - protein, 10 aa, Homo sapiens
  MOD_RES (8)..(8): PHOSPHORYLATION; tyrosine at position 8 is phosphorylated
SHEGETSYIR

SEQ ID NO: 261 - protein, 10 aa, Homo sapiens
  MOD_RES (5)..(5): PHOSPHORYLATION; tyrosine at position 5 is phosphorylated
EFEVYGPIKR

SEQ ID NO: 262 - protein, 26 aa, Homo sapiens
  MOD_RES (11)..(11): PHOSPHORYLATION; serine at position 11 is phosphorylated
KPPAPPSPVQSQSPSTNWSPAVPVKK

SEQ ID NO: 263 - protein, 26 aa, Homo sapiens
  MOD_RES (20)..(20): PHOSPHORYLATION; serine at position 20 is phosphorylated
GEGDAPFSEPGTTSTQRPSSPETATK

SEQ ID NO: 264 - protein, 15 aa, Homo sapiens
  MOD_RES (9)..(9): PHOSPHORYLATION; serine at position 9 is phosphorylated
VDNLTYRTSPDSLRR

SEQ ID NO: 265 - protein, 22 aa, Homo sapiens
  MOD_RES (10)..(10): PHOSPHORYLATION; serine at position 10 is phosphorylated
QSQQPMKPISPVKDPVSPASQK

SEQ ID NO: 266 - protein, 22 aa, Homo sapiens
  MOD_RES (17)..(17): PHOSPHORYLATION; serine at position 17 is phosphorylated
QSQQPMKPISPVKDPVSPASQK

SEQ ID NO: 267 - protein, 14 aa, Homo sapiens
  MOD_RES (7)..(7): PHOSPHORYLATION; tyrosine at position 7 is phosphorylated
LLFDDGYECDVLGK

SEQ ID NO: 268 - protein, 15 aa, Homo sapiens
  MOD_RES (7)..(7): PHOSPHORYLATION; tyrosine at position 7 is phosphorylated
VAAVGPYIQSSTMPR

SEQ ID NO: 269 - protein, 27 aa, Homo sapiens
  MOD_RES (6)..(6): PHOSPHORYLATION; tyrosine at position 6 is phosphorylated
TGASYYGEQTLHYIATNGESAVVQLPK

SEQ ID NO: 270 - protein, 30 aa, Homo sapiens
  MOD_RES (12)..(12): PHOSPHORYLATION; tyrosine at position 12 is phosphorylated
LISKPVASDSTYFAWCPDGEHILTATCAPR

SEQ ID NO: 271 - protein, 19 aa, Homo sapiens
  MOD_RES (9)..(9): PHOSPHORYLATION; serine at position 9 is phosphorylated
KTKPPRPDSPATTPNISVK

SEQ ID NO: 272 - protein, 16 aa, Homo sapiens
  MOD_RES (4)..(4): PHOSPHORYLATION; serine at position 4 is phosphorylated
RPHSPEKAFSSNPVVR

SEQ ID NO: 273 - protein, 20 aa, Homo sapiens
  MOD_RES (4)..(4): PHOSPHORYLATION; tyrosine at position 4 is phosphorylated
KEEYQGVIAWQGDYGEFVIK

SEQ ID NO: 274 - protein, 20 aa, Homo sapiens
  MOD_RES (14)..(14): PHOSPHORYLATION; tyrosine at position 14 is phosphorylated
KEEYQGVIAWQGDYGEFVIK

SEQ ID NO: 275 - protein, 28 aa, Homo sapiens
  MOD_RES (9)..(9): PHOSPHORYLATION; serine at position 9 is phosphorylated
VRGGAPDPSPGATATPGAPAQPSSPDAR

SEQ ID NO: 276 - protein, 26 aa, Homo sapiens
  MOD_RES (3)..(3): PHOSPHORYLATION; serine at position 3 is phosphorylated
SVSPVQDLDDDTPPSPAHSDMPYDAR

SEQ ID NO: 277 - protein, 26 aa, Homo sapiens
  MOD_RES (15)..(15): PHOSPHORYLATION; serine at position 15 is phosphorylated
SVSPVQDLDDDTPPSPAHSDMPYDAR

SEQ ID NO: 278 - protein, 29 aa, Homo sapiens
  MOD_RES (18)..(18): PHOSPHORYLATION; tyrosine at position 18 is phosphorylated
SVSSSPWLPQDGFDPSDYAEPMDAVVKPR

SEQ ID NO: 279 - protein, 26 aa, Homo sapiens
  MOD_RES (5)..(5): PHOSPHORYLATION; tyrosine at position 5 is phosphorylated
LATSYIAYLMDLLAKDDQNGEAEAFK

SEQ ID NO: 280 - protein, 26 aa, Homo sapiens
  MOD_RES (8)..(8): PHOSPHORYLATION; tyrosine at position 8 is phosphorylated
LATSYIAYLMDLLAKDDQNGEAEAFK

SEQ ID NO: 281 - protein, 25 aa, Homo sapiens
  MOD_RES (16)..(16): PHOSPHORYLATION; serine at position 16 is phosphorylated
ALSSAVQASPTSPGGSPSSPSSGQR

SEQ ID NO: 282 - protein, 35 aa, Homo sapiens
  MOD_RES (21)..(21): PHOSPHORYLATION; tyrosine at position 21 is phosphorylated
PFLQGGLPLGNLPSSSPMDSYPGLCQSPFLDSRER

SEQ ID NO: 283 - protein, 18 aa, Homo sapiens
  MOD_RES (15)..(15): PHOSPHORYLATION; tyrosine at position 15 is phosphorylated
VGDYVYFENSSSNPYLVR

SEQ ID NO: 284 - protein, 31 aa, Homo sapiens
  MOD_RES (18)..(18): PHOSPHORYLATION; tyrosine at position 18 is phosphorylated
TYLPANVPIIKTEPTDDYEPAPTCGPVSQGL

SEQ ID NO: 285 - protein, 23 aa, Homo sapiens
  MOD_RES (10)..(10): PHOSPHORYLATION; tyrosine at position 10 is phosphorylated
VGIYSPHTAYDAAPQGVNNWLAK

SEQ ID NO: 286 - protein, 21 aa, Homo sapiens
  MOD_RES (7)..(7): PHOSPHORYLATION; tyrosine at position 7 is phosphorylated
TTSSAIYMNLASHIQPGTVNR

SEQ ID NO: 287 - protein, 23 aa, Homo sapiens
  MOD_RES (12)..(12): PHOSPHORYLATION; serine at position 12 is phosphorylated
NSSTETDQQPHSPDSSSSVHSIR

SEQ ID NO: 288 - protein, 13 aa, Homo sapiens
  MOD_RES (11)..(11): PHOSPHORYLATION; tyrosine at position 11 is phosphorylated
KYGLFKEENPYAR

SEQ ID NO: 289 - protein, 19 aa, Homo sapiens
  MOD_RES (6)..(6): PHOSPHORYLATION; serine at position 6 is phosphorylated
KKQPASPDGRTSPINEDIR

SEQ ID NO: 290 - protein, 19 aa, Homo sapiens
  MOD_RES (12)..(12): PHOSPHORYLATION; serine at position  12 is phosphorylated
KKQPASPDGRTSPINEDIR

SEQ ID NO: 291 - protein, 28 aa, Homo sapiens
  MOD_RES (5)..(5): PHOSPHORYLATION; serine at position 5 is phosphorylated
YTPQSPTYTPSSPSYSPSSPSYSPTSPK

SEQ ID NO: 292 - protein, 28 aa, Homo sapiens
  MOD_RES (12)..(12): PHOSPHORYLATION; serine at position 12 is phosphorylated
YTPQSPTYTPSSPSYSPSSPSYSPTSPK

SEQ ID NO: 293 - protein, 21 aa, Homo sapiens
  MOD_RES (7)..(7): PHOSPHORYLATION; serine at position 7 is phosphorylated
YTPTSPSYSPSSPEYTPTSPK

SEQ ID NO: 294 - protein, 21 aa, Homo sapiens
  MOD_RES (12)..(12): PHOSPHORYLATION; serine at position 12 is phosphorylated
YTPTSPSYSPSSPEYTPTSPK

SEQ ID NO: 295 - protein, 25 aa, Homo sapiens
  MOD_RES (14)..(14): PHOSPHORYLATION; tyrosine at position 14 is phosphorylated
NVHTTDFPGNYSGYDDAWDQDRFEK

SEQ ID NO: 296 - protein, 21 aa, Homo sapiens
  MOD_RES (17)..(17): PHOSPHORYLATION; serine at position 17 is phosphorylated
RPQSPGASPSQAERLPSDSER

SEQ ID NO: 297 - protein, 29 aa, Homo sapiens
  MOD_RES (20)..(20): PHOSPHORYLATION; tyrosine at position 20 is phosphorylated
IIHTGEKPYKSKIMYTEENYKYEMKNVAK

SEQ ID NO: 298 - protein, 29 aa, Homo sapiens
  MOD_RES (22)..(22): PHOSPHORYLATION; tyrosine at position 22 is phosphorylated
IIHTGEKPYKSKIMYTEENYKYEMKNVAK

SEQ ID NO: 299 - protein, 33 aa, Homo sapiens
  MOD_RES (28)..(28): PHOSPHORYLATION; tyrosine at position 28 is phorphorylated
QQGHPNMGGPMQRMTPPRGMVPLGPQNYGGAMR

SEQ ID NO: 300 - protein, 18 aa, Homo sapiens
  MOD_RES (15)..(15): PHOSPHORYLATION; tyrosine at position 15 is phosphorylated
KREDDDDDDDDDDDYDNL

SEQ ID NO: 301 - protein, 25 aa, Homo sapiens
  MOD_RES (3)..(3): PHOSPHORYLATION; tyrosine at position 3 is phosphorylated
ISYTPPESPVPSYASSTPLHVPVPR

SEQ ID NO: 302 - protein, 25 aa, Homo sapiens
  MOD_RES (13)..(13): PHOSPHORYLATION; tyrosine at position 13 is phosphorylated
ISYTPPESPVPSYASSTPLHVPVPR

SEQ ID NO: 303 - protein, 10 aa, Homo sapiens
  MOD_RES (10)..(10): PHOSPHORYLATION; tyrosine at position 10 is phosphorylated
NLSHREDLAY

SEQ ID NO: 304 - protein, 22 aa, Homo sapiens
  MOD_RES (17)..(17): PHOSPHORYLATION; tyrosine at position 17 is phosphorylated
TDRLEHLESQELDEQIYQEDEC

SEQ ID NO: 305 - protein, 18 aa, Homo sapiens
  MOD_RES (7)..(7): PHOSPHORYLATION; serine at position 7 is phosphorylated
AGNWPGSPQVSGPSPAAR

SEQ ID NO: 306 - protein, 18 aa, Homo sapiens
  MOD_RES (14)..(14): PHOSPHORYLATION; serine at position 14 is phosphorylated
AGNWPGSPQVSGPSPAAR

SEQ ID NO: 307 - protein, 12 aa, Homo sapiens
  MOD_RES (11)..(11): PHOSPHORYLATION; tyrosine at position 11 is phosphorylated
QAYEPPPPPAYR

SEQ ID NO: 308 - protein, 21 aa, Homo sapiens
  MOD_RES (12)..(12): PHOSPHORYLATION; serine at position 12 is phosphorylated
NGDECAYHHPISPCKAFPNCK

SEQ ID NO: 309 - protein, 11 aa, Homo sapiens
  MOD_RES (10)..(10): PHOSPHORYLATION; tyrosine at position 10 is phosphorylated
KGTGLLSSDYR

SEQ ID NO: 310 - protein, 15 aa, Homo sapiens
  MOD_RES (11)..(11): PHOSPHORYLATION; serine at position 11 is phosphorylated
TEVSETARPSSPDTR

SEQ ID NO: 311 - protein, 13 aa, Homo sapiens
  MOD_RES (2)..(2): PHOSPHORYLATION; tyrosine at position 2 is phosphorylated
PYKCMECGKSYTR

SEQ ID NO: 312 - protein, 13 aa, Homo sapiens
  MOD_RES (11)..(11): PHOSPHORYLATION; tyrosine at position 11 is phosphorylated
PYKCMECGKSYTR

SEQ ID NO: 313 - protein, 18 aa, Homo sapiens
  MOD_RES (13)..(13): PHOSPHORYLATION; tyrosine at position 13 is phosphorylated
QTGGEEGDGASGYDAYWK

SEQ ID NO: 314 - protein, 18 aa, Homo sapiens
  MOD_RES (16)..(16): PHOSPHORYLATION; tyrosine at position 16 is phosphorylated
QTGGEEGDGASGYDAYWK

SEQ ID NO: 315 - protein, 28 aa, Homo sapiens
  MOD_RES (9)..(9): PHOSPHORYLATION; tyrosine at position 9 is phosphorylated
RKNSVKVMYKCLWPNCGKVLRSIVGIKR

SEQ ID NO: 316 - protein, 11 aa, Homo sapiens
  MOD_RES (4)..(4): PHOSPHORYLATION; tyrosine at position 4 is phosphorylated
EKLYDFVKTER

SEQ ID NO: 317 - protein, 28 aa, Homo sapiens
  MOD_RES (9)..(9): PHOSPHORYLATION; tyrosine at position 9 is phosphorylated
NSFNNPAYYVLEGVPHQLLPPEPPSPAR

SEQ ID NO: 318 - protein, 34 aa, Homo sapiens
  MOD_RES (21)..(21): PHOSPHORYLATION; serine at position 21 is phosphorylated
EAKPGAAEPEVGVPSSLSPSSPSSSWTETDVEER

SEQ ID NO: 319 - protein, 25 aa, Homo sapiens
  MOD_RES (20)..(20): PHOSPHORYLATION; tyrosine at position 20 is phosphorylated
GSKHWIIKNSWGESWGNKGYALLAR

SEQ ID NO: 320 - protein, 19 aa, Homo sapiens
  MOD_RES (14)..(14): PHOSPHORYLATION; tyrosine at posiiton 14 is phosphorylated
GLGEHEMEEDEEDYESSAK

SEQ ID NO: 321 - protein, 22 aa, Homo sapiens
  MOD_RES (10)..(10): PHOSPHORYLATION; tyrosine at position 10 is phosphorylated
KLAQQYYLVYQEPIPTAQLVQR

SEQ ID NO: 322 - protein, 14 aa, Homo sapiens
  MOD_RES (6)..(6): PHOSPHORYLATION; tyrosine at position 6 is phosphorylated
HIGLVYSGMGPDYR

SEQ ID NO: 323 - protein, 10 aa, Homo sapiens
  MOD_RES (6)..(6): PHOSPHORYLATION; tyrosine at position 6 is phosphorylated
TTTGSYIANR

SEQ ID NO: 324 - protein, 32 aa, Homo sapiens
  MOD_RES (5)..(5): PHOSPHORYLATION; serine at position 5 is phosphorylated
WTPKSPLDPDSGLLSCTLPNGFGGQSGPEGER

SEQ ID NO: 325 - protein, 36 aa, Homo sapiens
  MOD_RES (24)..(24): PHOSPHORYLATION; tyrosine at position 24 is phosphorylated
QGSGSSQPMEVQEGYGFGSGDDPYSSAEPHVSGVKR

SEQ ID NO: 326 - protein, 8 aa, Homo sapiens
  MOD_RES (4)..(4): PHOSPHORYLATION; tyrosine at position 4 is phosphorylated
VYTYIQSR

SEQ ID NO: 327 - protein, 38 aa, Homo sapiens
  MOD_RES (27)..(27): PHOSPHORYLATION; tyrosine at position 27 is phosphorylated
TGQIWPNDGEGAFHGDADGSFGTPPGYGCAADRAEEQR

SEQ ID NO: 328 - protein, 15 aa, Homo sapiens
  MOD_RES (6)..(6): PHOSPHORYLATION; tyrosine at position 6 is phosphorylated
AFVDNYGVAMEMAEK

SEQ ID NO: 329 - protein, 36 aa, Homo sapiens
  MOD_RES (22)..(22): PHOSPHORYLATION; tyrosine at position 22 is phosphorylated
LQTVHSIPLTINKEDDESPGLYGFLNVIVHSATGFK

SEQ ID NO: 330 - protein, 22 aa, Homo sapiens
  MOD_RES (9)..(9): PHOSPHORYLATION; serine at position 9 is phosphorylated
CICPSLPYSPVSSPQSSPRLPR

SEQ ID NO: 331 - protein, 22 aa, Homo sapiens
  MOD_RES (13)..(13): PHOSPHORYLATION; serine at position 13 is phosphorylated
CICPSLPYSPVSSPQSSPRLPR

SEQ ID NO: 332 - protein, 22 aa, Homo sapiens
  MOD_RES (16)..(16): PHOSPHORYLATION; serine at position 16 is phosphorylated
CICPSLPYSPVSSPQSSPRLPR

SEQ ID NO: 333 - protein, 17 aa, Homo sapiens
  MOD_RES (13)..(13): PHOSPHORYLATION; tyrosine at position 13 is phosphorylated
NLSEGNNANYTEYVATR

SEQ ID NO: 334 - protein, 20 aa, Homo sapiens
  MOD_RES (12)..(12): PHOSPHORYLATION; tyrosine at position 12 is phosphorylated
KKPHASVGTHGYMAPEVLQK

SEQ ID NO: 335 - protein, 13 aa, Homo sapiens
  MOD_RES (4)..(4): PHOSPHORYLATION; tyrosine at position 4 is phosphorylated
VTIYSFTGNQRNR

SEQ ID NO: 336 - protein, 27 aa, Homo sapiens
  MOD_RES (13)..(13): PHOSPHORYLATION; tyrosine at position 13 is phosphorylated
ETTSHNSLTTPCYTPYYVAPEVLGPEK

SEQ ID NO: 337 - protein, 27 aa, Homo sapiens
  MOD_RES (16)..(16): PHOSPHORYLATION; tyrosine at position 16 is phosphorylated
ETTSHNSLTTPCYTPYYVAPEVLGPEK

SEQ ID NO: 338 - protein, 27 aa, Homo sapiens
  MOD_RES (17)..(17): PHOSPHORYLATION; tyrosine at position 17 is phosphorylated
ETTSHNSLTTPCYTPYYVAPEVLGPEK

SEQ ID NO: 339 - protein, 29 aa, Homo sapiens
  MOD_RES (12)..(12): PHOSPHORYLATION; tyrosine at position 12 is phosphorylated
ETTQNALQTPCYTPYYVAPEVLGPEKYDK

SEQ ID NO: 340 - protein, 29 aa, Homo sapiens
  MOD_RES (15)..(15): PHOSPHORYLATION; tyrosine at position 15 is phosphorylated
ETTQNALQTPCYTPYYVAPEVLGPEKYDK

SEQ ID NO: 341 - protein, 29 aa, Homo sapiens
  MOD_RES (16)..(16): PHOSPHORYLATION; tyrosine at position 16 is phosphorylated
ETTQNALQTPCYTPYYVAPEVLGPEKYDK

SEQ ID NO: 342 - protein, 21 aa, Homo sapiens
  MOD_RES (13)..(13): PHOSPHORYLATION; serine at position 13 is phosphorylated
DQQNLPYGVTPASPSGHSQGR

SEQ ID NO: 343 - protein, 12 aa, Homo sapiens
  MOD_RES (3)..(3): PHOSPHORYLATION; tyrosine at position 3 is phosphorylated
GTYGKVYKVTNK

SEQ ID NO: 344 - protein, 17 aa, Homo sapiens
  MOD_RES (16)..(16): PHOSPHORYLATION; tyrosine at position 16 is phosphorylated
KADSYEKLEKLGEGSYA

SEQ ID NO: 345 - protein, 16 aa, Homo sapiens
  MOD_RES (4)..(4): PHOSPHORYLATION; tyrosine at position 4 is phosphorylated
VLGYNHNGEWCEAQTK

SEQ ID NO: 346 - protein, 18 aa, Homo sapiens
  MOD_RES (5)..(5): PHOSPHORYLATION; tyrosine at position 5 is phosphorylated
NAAEYLLSSGINGSFLVR

SEQ ID NO: 347 - protein, 11 aa, Homo sapiens
  MOD_RES (9)..(9): PHOSPHORYLATION; tyrosine at position 9 is phosphorylated
WTAPESLAYNK

SEQ ID NO: 348 - protein, 21 aa, Homo sapiens
  MOD_RES (12)..(12): PHOSPHORYLATION; tyrosine at position 12 is phosphorylated
SKNGQGWVPSNYITPVNSLEK

SEQ ID NO: 349 - protein, 20 aa, Homo sapiens
  MOD_RES (7)..(7): PHOSPHORYLATION; tyrosine at position 7 is phosphorylated
CNKPTVYGVSPIHDKWEMER

SEQ ID NO: 350 - protein, 18 aa, Homo sapiens
  MOD_RES (12)..(12): PHOSPHORYLATION; tyrosine at position 12 is phosphorylated
HKLGGGQYGEVYVGVWKK

SEQ ID NO: 351 - protein, 9 aa, Homo sapiens
  MOD_RES (8)..(8): PHOSPHORYLATION; tyrosine at position 8 is phosphorylated
YVGVWKKYS

SEQ ID NO: 352 - protein, 20 aa, Homo sapiens
  MOD_RES (10)..(10): PHOSPHORYLATION; tyrosine at position 10 is phosphorylated
AASSSSVVPYLPRLPILPSK

SEQ ID NO: 353 - protein, 15 aa, Homo sapiens
  MOD_RES (14)..(14): PHOSPHORYLATION; tyrosine at position 14 is phosphorylated
SSFREMENQPHKKYE

SEQ ID NO: 354 - protein, 40 aa, Homo sapiens
  MOD_RES (8)..(8): PHOSPHORYLATION; tyrosine at position 8 is phosphorylated
NLVPPKCYGGSFAQRNLCNDDGGGGGGSGTAGGGWSGITG

SEQ ID NO: 355 - protein, 12 aa, Homo sapiens
  MOD_RES (8)..(8): PHOSPHORYLATION; tyrosine at position 8 is phosphorylated
CLDEGGYYISPR

SEQ ID NO: 356 - protein, 13 aa, Homo sapiens
  MOD_RES (6)..(6): PHOSPHORYLATION; tyrosine at position 6 is phosphorylated
IIDSEYTAQEGAK

SEQ ID NO: 357 - protein, 19 aa, Homo sapiens
  MOD_RES (8)..(8): PHOSPHORYLATION; tyrosine at position 8 is phosphorylated
KVVALYDYMPMNANDLQLR

SEQ ID NO: 358 - protein, 23 aa, Homo sapiens
  MOD_RES (12)..(12): PHOSPHORYLATION; tyrosine at position 12 is phosphorylated
HLFSTIPELINYHQHNSAGLISR

SEQ ID NO: 359 - protein, 14 aa, Homo sapiens
  MOD_RES (2)..(2): PHOSPHORYLATION; tyrosine at position 2 is phosphorylated
SYGAADHYGPDPTK

SEQ ID NO: 360 - protein, 14 aa, Homo sapiens
  MOD_RES (8)..(8): PHOSPHORYLATION; tyrosine at position 8 is phosphorylated
SYGAADHYGPDPTK

SEQ ID NO: 361 - protein, 12 aa, Homo sapiens
  MOD_RES (8)..(8): PHOSPHORYLATION; tyrosine at position 8 is phosphorylated
KLDNGGYYITTR

SEQ ID NO: 362 - protein, 12 aa, Homo sapiens
  MOD_RES (7)..(7): PHOSPHORYLATION; tyrosine at position 7 is phosphorylated
SLDNGGYYISPR

SEQ ID NO: 363 - protein, 19 aa, Homo sapiens
  MOD_RES (13)..(13): PHOSPHORYLATION; tyrosine at position 13 is phosphorylated
LGAGQFGEVWMGYYNNSTK

SEQ ID NO: 364 - protein, 11 aa, Homo sapiens
  MOD_RES (3)..(3): PHOSPHORYLATION; tyrosine at position 3 is phosphorylated
TIYVRDPTSNK

SEQ ID NO: 365 - protein, 13 aa, Homo sapiens
  MOD_RES (9)..(9): PHOSPHORYLATION; tyrosine at position 9 is phosphorylated
VENCPDELYDIMK

SEQ ID NO: 366 - protein, 13 aa, Homo sapiens
  MOD_RES (11)..(11): PHOSPHORYLATION; tyrosine at position 11 is phosphorylated
LLAQAEGEPCYIR

SEQ ID NO: 367 - protein, 15 aa, Homo sapiens
  MOD_RES (12)..(12): PHOSPHORYLATION; tyrosine at position 12 is phosphorylated
EAQIMHQLDNPYIVR

SEQ ID NO: 368 - protein, 16 aa, Homo sapiens
  MOD_RES (10)..(10): PHOSPHORYLATION; tyrosine at position 10 is phosphorylated
VLEDDPEATYTTSGGK

SEQ ID NO: 369 - protein, 11 aa, Homo sapiens
  MOD_RES (8)..(8): PHOSPHORYLATION; tyrosine at position 8 is phosphorylated
DIMSDSNYVVR

SEQ ID NO: 370 - protein, 30 aa, Homo sapiens
  MOD_RES (24)..(24): PHOSPHORYLATION; tyrosine at position 24 is phosphorylated
KRPSFPNLTSFLGCQLADAEEAMYQNVDGR

SEQ ID NO: 371 - protein, 11 aa, Homo sapiens
  MOD_RES (8)..(8): PHOSPHORYLATION; tyrosine at position 8 is phosphorylated
VSECPHTYQNR

SEQ ID NO: 372 - protein, 11 aa, Homo sapiens
  MOD_RES (1)..(1): PHOSPHORYLATION; tyrosine at position 1 is phosphorylated
YKDVVAYDETR

SEQ ID NO: 373 - protein, 16 aa, Homo sapiens
  MOD_RES (11)..(11): PHOSPHORYLATION; tyrosine at position 11 is phosphorylated
GQESEYGNITYPPAMK

SEQ ID NO: 374 - protein, 15 aa, Homo sapiens
  MOD_RES (8)..(8): PHOSPHORYLATION; tyrosine at position 8 is phosphorylated
IQNSGDFYDLYGGEK

SEQ ID NO: 375 - protein, 15 aa, Homo sapiens
  MOD_RES (11)..(11): PHOSPHORYLATION; tyrosine at position 11 is phosphorylated
IQNSGDFYDLYGGEK

SEQ ID NO: 376 - protein, 13 aa, Homo sapiens
  MOD_RES (1)..(1): PHOSPHORYLATION; tyrosine at position 1 is phosphorylated
YLLEQLEKWTEYR

SEQ ID NO: 377 - protein, 13 aa, Homo sapiens
  MOD_RES (12)..(12): PHOSPHORYLATION; tyrosine at position 12 is phosphorylated
YLLEQLEKWTEYR

SEQ ID NO: 378 - protein, 15 aa, Homo sapiens
  MOD_RES (1)..(1): PHOSPHORYLATION; tyrosine at position 1 is phosphorylated
YRNIGSETWKTIITK

SEQ ID NO: 379 - protein, 15 aa, Homo sapiens
  MOD_RES (14)..(14): PHOSPHORYLATION; tyrosine at position 14 is phosphorylated
TPLPLCSSQAQMDYR

SEQ ID NO: 380 - protein, 31 aa, Homo sapiens
  MOD_RES (3)..(3): PHOSPHORYLATION; tyrosine at position 3 is phosphorylated
VSYVGCMVQYSVALALGSTECVLLAIMAVDR

SEQ ID NO: 381 - protein, 12 aa, Homo sapiens
  MOD_RES (9)..(9): PHOSPHORYLATION; tyrosine at position 9 is phosphorylated
WPTVDASYYGGR

SEQ ID NO: 382 - protein, 23 aa, Homo sapiens
  MOD_RES (15)..(15): PHOSPHORYLATION; tyrosine at position 15 is phosphorylated
NLEYVSVSPTNNTVYASVTHSNR

SEQ ID NO: 383 - protein, 15 aa, Homo sapiens
  MOD_RES (4)..(4): PHOSPHORYLATION; tyrosine at position 4 is phosphorylated
SDVYSDLNTQRPYYK

SEQ ID NO: 384 - protein, 15 aa, Homo sapiens
  MOD_RES (13)..(13): PHOSPHORYLATION; tyrosine at position 13 is phosphorylated
SDVYSDLNTQRPYYK

SEQ ID NO: 385 - protein, 15 aa, Homo sapiens
  MOD_RES (14)..(14): PHOSPHORYLATION; tyrosine at position 14 is phosphorylated
SDVYSDLNTQRPYYK

SEQ ID NO: 386 - protein, 15 aa, Homo sapiens
  MOD_RES (8)..(8): PHOSPHORYLATION; tyrosine at position 8 is phosphorylated
ITETESPYQELQGQR

SEQ ID NO: 387 - protein, 15 aa, Homo sapiens
  MOD_RES (6)..(6): PHOSPHORYLATION; tyrosine at position 6 is phosphorylated
FVTSMYNEILILGAK

SEQ ID NO: 388 - protein, 20 aa, Homo sapiens
  MOD_RES (4)..(4): PHOSPHORYLATION; tyrosine at position 4 is phosphorylated
ARQYTSPEEIDAQLQAEKQK

SEQ ID NO: 389 - protein, 32 aa, Homo sapiens
  MOD_RES (15)..(15): PHOSPHORYLATION; tyrosine at position 15 is phosphorylated
RVVLGDGVQLPPGDYSTTPGGTLFSTTPGGTR

SEQ ID NO: 390 - protein, 12 aa, Homo sapiens
  MOD_RES (7)..(7): PHOSPHORYLATION; tyrosine at position 7 is phosphorylated
EHALLAYTLGVK

SEQ ID NO: 391 - protein, 34 aa, Homo sapiens
  MOD_RES (23)..(23): PHOSPHORYLATION; tyrosine at position 23 is phosphorylated
LKAERERGITIDISLWKFETSKYYVTIIDAPGHR

SEQ ID NO: 392 - protein, 24 aa, Homo sapiens
  MOD_RES (4)..(4): PHOSPHORYLATION; serine at position 4 is phosphorylated
TCFSPNRVIGLSSDLQQVGGASAR

SEQ ID NO: 393 - protein, 22 aa, Homo sapiens
  MOD_RES (5)..(5): PHOSPHORYLATION; tyrosine at position 5 is phosphorylated
QDLAYERQYEQQTYQVIPEVIK

SEQ ID NO: 394 - protein, 22 aa, Homo sapiens
  MOD_RES (9)..(9): PHOSPHORYLATION; tyrosine at position 9  is phosphorylated
QDLAYERQYEQQTYQVIPEVIK

SEQ ID NO: 395 - protein, 22 aa, Homo sapiens
  MOD_RES (14)..(14): PHOSPHORYLATION; tyrosine at position 14 is phosphorylated
QDLAYERQYEQQTYQVIPEVIK

SEQ ID NO: 396 - protein, 14 aa, Homo sapiens
  MOD_RES (11)..(11): PHOSPHORYLATION; tyrsoine at position 11 is phosphorylated
ARPATDSFDDYPPR

SEQ ID NO: 397 - protein, 9 aa, Homo sapiens
  MOD_RES (3)..(3): PHOSPHORYLATION; tyrosine at position 3 is phosphorylated
DDYSRDDYR

SEQ ID NO: 398 - protein, 10 aa, Homo sapiens
  MOD_RES (8)..(8): PHOSPHORYLATION; tyrosine at position 8 is phosphorylated
DDYSRDDYRR

SEQ ID NO: 399 - protein, 28 aa, Homo sapiens
  MOD_RES (4)..(4): PHOSPHORYLATION; tyrosine at position 4 is phosphorylated
KFAYLGRLAHEVGWKYQAVTATLEEKRK

SEQ ID NO: 400 - protein, 28 aa, Homo sapiens
  MOD_RES (16)..(16): PHOSPHORYLATION; tyrosine at position 16 is phosphorylated
KFAYLGRLAHEVGWKYQAVTATLEEKRK

SEQ ID NO: 401 - protein, 17 aa, Homo sapiens
  MOD_RES (4)..(4): PHOSPHORYLATION; tyrosine at position 4 is phosphorylated
SNYYEGPHTSHSSPADR

SEQ ID NO: 402 - protein, 35 aa, Homo sapiens
  MOD_RES (4)..(4): PHOSPHORYLATION; tyrosine at position 4 is phosphorylated
GDDYFNYNVQQTSTNPPLPEPGYFTKPPIAAHASR

SEQ ID NO: 403 - protein, 35 aa, Homo sapiens; identical to SEQ ID NO: 402
  MOD_RES (23)..(23): PHOSPHORYLATION; tyrosine at position 23 is phosphorylated

SEQ ID NO: 404 - protein, 20 aa, Homo sapiens
  MOD_RES (1)..(1): PHOSPHORYLATION; tyrosine at position 1 is phosphorylated
YQEIVTLVLFIIMALLWFSR

SEQ ID NO: 405 - protein, 31 aa, Homo sapiens
  MOD_RES (9)..(9): PHOSPHORYLATION; tyrosine at position 9 is phosphorylated
IQSNKGSSYKLLVEQAKLKQATSKHFKDLIR

SEQ ID NO: 406 - protein, 31 aa, Homo sapiens
  MOD_RES (28)..(28): PHOSPHORYLATION; tyrosine at position 28 is phosphorylated
VPNSSVNQTEPQVSSSHNPTSTEEQQLYWAK

SEQ ID NO: 407 - protein, 27 aa, Homo sapiens
  MOD_RES (21)..(21): PHOSPHORYLATION; serine at position 21 is phosphorylated
APDSGLPSGGGGRPGCAYPGSPGPGAR

SEQ ID NO: 408 - protein, 15 aa, Homo sapiens
  MOD_RES (3)..(3): PHOSPHORYLATION; tyrosine at position 3 is phosphorylated
FIYGNQDLFATSQSK

SEQ ID NO: 409 - protein, 11 aa, Homo sapiens
  MOD_RES (10)..(10): PHOSPHORYLATION; tyrosine at position 10 is phosphorylated
RGILQTLNVYL

SEQ ID NO: 410 - protein, 16 aa, Homo sapiens
  MOD_RES (7)..(7): PHOSPHORYLATION; serine at position 7 is phosphorylated
SRLTPVSPESSSTEEK

SEQ ID NO: 411 - protein, 17 aa, Homo sapiens
  MOD_RES (6)..(6): PHOSPHORYLATION; tyrosine at position 6 is phosphorylated
TSIDAYDNFDNISLAQR

SEQ ID NO: 412 - protein, 18 aa, Homo sapiens
  MOD_RES (14)..(14): PHOSPHORYLATION; tyrosine at position 14 is phosphorylated
GLLQRALEHFTDLYDIKR

SEQ ID NO: 413 - protein, 21 aa, Homo sapiens
  MOD_RES (8)..(8): PHOSPHORYLATION; tyrosine at position 8 is phosphorylated
LVTEMGTYATQSALSSSRPTK

SEQ ID NO: 414 - protein, 9 aa, Homo sapiens
  MOD_RES (2)..(2): PHOSPHORYLATION; tyrosine at position 2 is phosphorylated
GYDNPSFLR

SEQ ID NO: 415 - protein, 29 aa, Homo sapiens
  MOD_RES (6)..(6): PHOSPHORYLATION; tyrosine at position 6 is phosphorylated
VVLPSYEEALSLPSKTPEGGPAPPPYSEV

SEQ ID NO: 416 - protein, 29 aa, Homo sapiens
  MOD_RES (26)..(26): PHOSPHORYLATION; tyrosine at position 26 is phosphorylated
VVLPSYEEALSLPSKTPEGGPAPPPYSEV

SEQ ID NO: 417 - protein, 14 aa, Homo sapiens
  MOD_RES (3)..(3): PHOSPHORYLATION; tyrosine at position 3 is phosphorylated
WPYQNGFTLNTWFR

SEQ ID NO: 418 - protein, 17 aa, Homo sapiens
  MOD_RES (11)..(11): PHOSPHORYLATION; tyrosine at position 11 is phosphorylated
LGAAPEEESAYVAGEKR

SEQ ID NO: 419 - protein, 25 aa, Homo sapiens
  MOD_RES (19)..(19): PHOSPHORYLATION; tyrosine at position 19 is phosphorylated
DLIHDQDEDEEEEEGQRFYAGGSER

SEQ ID NO: 420 - protein, 28 aa, Homo sapiens
  MOD_RES (17)..(17): PHOSPHORYLATION; tyrosine at position  17 is phosphorylated
LCVVLGPYGPEWQENPYPFQCTIDDPTK

SEQ ID NO: 421 - protein, 15 aa, Homo sapiens
  MOD_RES (2)..(2): PHOSPHORYLATION; tyrosine at position 2 is phosphorylated
RYANVPPGGFEPLPV

SEQ ID NO: 422 - protein, 37 aa, Homo sapiens
  MOD_RES (6)..(6): PHOSPHORYLATION; tyrosine at position 6 is phosphorylated
TTAGTYSSPPPASYSTLQAPALSVTGPITANSEQIAR

SEQ ID NO: 423 - protein, 37 aa, Homo sapiens; identical to SEQ ID NO: 422
  MOD_RES (14)..(14): PHOSPHORYLATION; tyrosine at position  14 is phosphorylated

SEQ ID NO: 424 - protein, 32 aa, Homo sapiens
  MOD_RES (29)..(29): PHOSPHORYLATION; tyrosine at position 29 is phosphorylated
NAQALQQMLKHPLLCHSSKPKLDTLQKPYVHK